(19) United States
(12) Patent Application Publication
(10) Pub. No.: US 2006/0282908 A1
(43) Pub. Date: Dec. 14, 2006

(54) Identification of the genetic determinants of the polymorphic CYP3A5 expression

(76) Inventors: Leszek Wojnowski, Gottingen (DE); Elisabeth Hustert, Germering (DE); Michael Haberl, Bernried (DE)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/607,596

(22) Filed: Jun. 27, 2003

Related U.S. Application Data
(63) Continuation of application No. PCT/EP01/15290, filed on Dec. 21, 2001.
(60) Provisional application No. 60/258,684, filed on Dec. 28, 2000.
(60) Provisional application No. 60/258,952, filed on Dec. 29, 2000.
(60) Provisional application No. 60/262,859, filed on Jan. 18, 2001.
(60) Provisional application No. 60/312,825, filed on Aug. 16, 2001.

(30) Foreign Application Priority Data
Dec. 28, 2000 (EP) 00128627.7
Jan. 16, 2001 (EP) 01100172.4
Aug. 16, 2001 (EP) 01118884.4

(51) Int. Cl. A01K 67/027; C12Q 1/68; C07H 21/04; C12P 21/06; C12N 9/02

(52) U.S. Cl. 800/14; 435/6; 435/69.1; 435/320.1; 435/353; 435/354; 435/189; 800/18; 800/20; 536/23.2

(57) ABSTRACT

The present invention relates to a polymorphic CYP3A5 polynucleotide. Moreover, the invention relates to genes or vectors comprising the polynucleotides of the invention and to a host cell genetically engineered with the polynucleotide or gene of the invention. Further, the invention relates to methods for producing molecular variant polypeptides or fragments thereof, methods for producing cells capable of expressing a molecular variant polypeptide and to a polypeptide or fragment thereof encoded by the polynucleotide or the gene of the invention or which is obtainable by the method or from the cells produced by the method of the invention. Furthermore, the invention relates to an antibody which binds specifically the polypeptide of the invention. Moreover, the invention relates to a transgenic non-human animal. The invention also relates to a solid support comprising one or a plurality of the above mentioned polynucleotides, genes, vectors, polypeptides, antibodies or host cells. Furthermore, methods of identifying a polymorphism, identifying and obtaining a pro-drug or drug or an inhibitor are also encompassed by the present invention. In addition, the invention relates to methods for producing of a pharmaceutical composition and to methods of diagnosing a disease. Further, the invention relates to a method of detection of the polynucleotide of the invention. Furthermore, comprised by the present invention are a diagnostic and a pharmaceutical composition. Even more, the invention relates to uses of the polynucleotides, genes, vectors, polypeptides or antibodies of the invention. Finally, the invention relates to a diagnostic kit.

The present invention relates to a polymorphic CYP3A5 polynucleotide. Moreover, the invention relates to genes or vectors comprising the polynucleotides of the invention and to a host cell genetically engineered with the polynucleotide or gene of the invention. Further, the invention relates to methods for producing molecular variant polypeptides or fragments thereof, methods for producing cells capable of expressing a molecular variant polypeptide and to a polypeptide or fragment thereof encoded by the polynucleotide or the gene of the invention or which is obtainable by the method or from the cells produced by the method of the invention. Furthermore, the invention relates to an antibody which binds specifically the polypeptide of the invention. Moreover, the invention relates to a transgenic non-human animal. The invention also relates to a solid support comprising one or a plurality of the above mentioned polynucleotides, genes, vectors, polypeptides, antibodies or host cells. Furthermore, methods of identifying a polymorphism, identifying and obtaining a pro-drug or drug or an inhibitor are also encompassed by the present invention. In addition, the invention relates to methods for producing of a pharmaceutical composition and to methods of diagnosing a disease. Further, the invention relates to a method of detection of the polynucleotide of the invention. Furthermore, comprised by the present invention are a diagnostic and a pharmaceutical composition. Even more, the invention relates to uses of the polynucleotides, genes, vectors, polypeptides or antibodies of the invention. Finally, the invention relates to a diagnostic kit.

The CYP3A enzymes play a particularly important role in drug metabolism. This is due to their abundant expression in the liver combined with a broad substrate spectrum. Indeed, it is estimated that CYP3A isozymes collectively comprise the largest portion of the liver CYP protein (Thummel, Annu Rev Pharmacol Toxicol 38 (1998), 389-430) and that they are involved in the metabolism of 45%-60% of all currently used drugs (Li, Toxicology 104 (1995), 1-8; Evans, Science 286 (1999), 487-91). In addition to drugs, CYP3A isozymes metabolise a variety of other compounds including steroid hormones, toxins and carcinogens. For example, CYP3A isozymes metabolise aflatoxin B, (Wang, Biochemistry 37 (1998), 12536-45; Gillam, Arch Biochem Biophys 317 (1995), 374-84; Li, Cancer Res 57 (1997), 641-5), a mycotoxin strongly implicated in the etiology of liver cancer, which is a major cause of premature death in many areas of Africa and Asia (Henry, Science 286 (1999), 2453-4).

The hepatic expression and activity of CYP3A isozymes is inter-individually variable and this variability is the reason for harmful interactions frequently encountered in development and application of drugs that are CYP3A substrates. It has also been postulated that variable CYP3A expression could affect an individual's predisposition to cancers caused by environmental carcinogens which are metabolised by CYP3A. The elucidation of factors controlling an individual's CYP3A activity could permit personalised dose adjustments in therapies with its substrates and also lead to the identification of sub-populations at increased risk for several common cancers. However, despite considerable efforts, our understanding of factors governing CYP3A activity and expression is limited. There are several reasons for this: An average human liver may express products of up to four CYP3A genes (Gellner, Pharmacogenetics 11 (2001), 111-121), but their respective contributions to the hepatic CYP3A pool are still a matter of debate. The differentiation between the individual CYP3A proteins by enzymatic methods has proven difficult due to overlapping substrate specifities and due to the considerable effect of reconstitution conditions on their catalytic activities. RNA and protein analysis indicate that CYP3A4 forms the bulk of the hepatic CYP3A protein and its expression is highly variable (Thummel, Annu Rev Pharmacol Toxicol 38 (1998), 389-430). Less well understood are the contributions of the other CYP3A genes. CYP3A5 is widely considered the second most important CYP3A protein in the liver, but the available data are conflicting, since its expression has been reported to be present in 10% to 97% of human livers (Aoyama, J Biol Chem 264 (1989), 10388-95; Wrighton, Mol Pharmacol 38 (1990), 207-13; Schuetz, Pharmacogenetics 4 (1994), 11-20; Jounaidi, Biochem Biophys Res Commun 221 (1996), 466-70; Boobis, Br J Clin Pharmacol 42 (1996), 81-9). The possible reasons for these discrepancies include small sample sizes, interethnic differences and poor specificity of probes used to measure CYP3A5 expression. The third CYP3A, CYP3A7, was originally described in the human fetal liver where it accounts for about 50% of the total CYP protein (Wrighton, Biochem Pharmacol 37 (1988), 3053-5). More recent studies indicate constitutive or induced expression of CYP3A7 in adult human livers, but its quantification has been hampered by the lack of specific antibodies. Similarly, no protein expression data are available for the recently identified fourth member of the family, CYP3A43 (Gellner, Pharmacogenetics 11 (2001), 111-121).

Clinical studies indicate that a major portion of the inter-individual CYP3A variability is caused by genetic factors (Ozdemir, Pharmacogenetics 10 (2000), 373-88), but the identities of the latter remain unknown. In respect of CYP3A5, a protein variant (Thr398Asn) has been found in 2 out of 5 individuals deficient in CYP3A5 expression (Jounaidi, Biochem Biophys Res Commun 221 (1996), 466-70), but its significance has not been verified on a larger number of liver samples and in functional studies. In addition, a haplotype consisting of two linked polymorphisms has been described in the 5′ flanking region of the CYP3A5 gene which is associated with increased expression and activity of the gene (Paulussen, Pharmacogenetics 10 (2000), 415-24). However, only a small sample set (n=29) was analysed for the genotype and the phenotype. Moreover, the single nucleotide polymorphisms (SNPs) which have been disclosed in said document are not suitable for a reliable prediction of CYP3A5 dysfunction and/or dysregulation and the problems caused thereby. This document does not suggest the existence of further haplotypes.

Thus, improved means and methods for diagnosing and treating a variety of diseases and disorders based on dysfunctions or dysregulations of drug metabolism were not available yet but are nevertheless highly desirable. Thus, the technical problem underlying the present invention is to comply with the above specified needs.

The solution to this technical problem is achieved by providing the embodiments characterized in the claims.

Accordingly, the present invention relates to a polynucleotide comprising a polynucleotide selected from the group consisting of:
(a) a polynucleotide having the nucleic acid sequence of SEQ ID NO: 54, 56, 58, 62, 64, 66, 68, 70, 72, 74, 76, 78, 80, 82, 84, 86, 88, 90, 92, 94, 96, 98, 100, 102, 106, 108, 110, 112, 113, 114, 115, 116, 117, 118, 119, 120, 121, 122, 123, 124, 125, 126, 128, 129, 130, 131, 133, 134, 135, 136, 137, 138, 139, 140, 142, 143, 149, 151, 153, 155, 157, 159, 161, 163, 165, 169, 171, 173, 175, 177, 179, 181, 183, 185, 187, 189, 193, 195, 197, 199, 201, 207, 208, 209, 210, 211, 212, 213, 214, 216, 218, 219, 220, 221, 222, 223, 224, 225, 226, 227, 228, 231, 232, 233, 235, or 236;
(b) a polynucleotide encoding a polypeptide having the amino acid sequence of SEQ ID NO: 127, 132, 141, 215, 229, or 234;
(c) a polynucleotide capable of hybridizing to a CYP3A5 gene, wherein said polynucleotide is having a nucleotide exchange, a nucleotide deletion of at least one nucleotide, or at least one additional nucleotide at a position corresponding to position -20643, -20555, -20359, -20367, -20329, -20323, -20310, -6200, -6177, -4336, -3990, -3868, -3844, -3557, -1617, -795, -86, -74, 136, 174 to 176, 230, 3705, 3709/3710, 5215, 5235, 5516, 7182, 7207, 7303, 7424/7427, 12907, 13028, 13077, 13173, 13226, 13376, 14720, 14836, 14903, 15788, 16079, 16931/16932, 16993, 17163, 19069, 19165, 19208, 27050, 27131/27132, 27526, 31499, 31551 or 3161.1 of the CYP3A5 gene (Accession No: AF280107.1, wherein position 166220 has been numbered +1 and position 174832 has been numbered +8613, and Accession No: AC005020.2, wherein position 27341 has been numbered +8614);
(d) a polynucleotide capable of hybridizing to a CYP3A5 gene, wherein said polynucleotide is having an A at a position corresponding to position -20555, -20329, -20323, -4336, -3868, -3844, -795, -86, 230, 5235, 5516, 7182, 7303, 12907, 13028, 13376, 19069 or 19165 of the CYP3A5 gene (Accession No: AF280107.1, wherein position 166220 has been numbered +1 and position 174832 has been numbered +8613, and Accession No: AC005020.2, wherein position 27341 has been numbered +8614), a T at a position corresponding to position -20367, -6200, -74, 3705, 5215, 7207, 14836, 17163, 19208 or 27526 of the CYP3A5 gene (Accession No: AF280107.1, wherein position 166220 has been numbered +1 and position 174832 has been numbered +8613, and Accession No: AC005020.2, wherein position 27341 has been numbered +8614), a G at a position corresponding to position -6177, -3990, 13077, 14720, 14903, 16993 or 27050 of the CYP3A5 gene (Accession No: AF280107.1, wherein position 166220 has been numbered +1 and position 174832 has been numbered +8613, and Accession No: AC005020.2, wherein position 27341 has been numbered +8614), a C at a position corresponding to position -20643, -20310, -3557, -1617, 136, 13173, 13226, 15788, 16079, 31499, 31551 or 31611 of the CYP3A5 gene (Accession No: AF280107.1, wherein position 166220 has been numbered +1 and position 174832 has been numbered +8613, and Accession No: AC005020.2, wherein position 27341 has been numbered +8614), nucleotide deletions at positions corresponding to positions 174 to 176 of the CYP3A5 gene (Accession No: AF280107.1, wherein position 166220 has been numbered +1 and position 174832 has been numbered +8613), an additional nucleotide at a position corresponding to position 3709/3710 or 27131/27132 of the CYP3A5 gene (Accession No: AF280107.1, wherein position 166220 has been numbered +1 and position 174832 has been numbered +8613, and Accession No: AC005020.2, wherein position 27341 has been numbered +8614), three additional nucleotides at a position corresponding to position 16931/16932 of the CYP3A5 gene (Accession No: AF280107.1, wherein position 166220 has been numbered +1 and position 174832 has been numbered +8613, and Accession No: AC005020.2, wherein position 27341 has been numbered +8614), or a deletion of two nucleotides and nine additional nucleotides inserted at a position corresponding to position 7424 to 7427 of the CYP3A5 gene (Accession No: AF280107.1, wherein position 166220 has been numbered +1 and position 174832 has been numbered +8613);
(e) a polynucleotide encoding a CYP3A5 polypeptide or fragment thereof, wherein said polypeptide comprises an amino acid substitution at a position corresponding to position 30, 100, 130, 149 or 488 of the CYP3A5 polypeptide (Accession No: NP_{—}000768.1), or at least one amino acid exchange or a stop codon at a position corresponding to position 30 to 34 or 346 to 348 of the CYP3A5 polypeptide (Accession No: NP_{—}000768.1); and
(f) a polynucleotide encoding a CYP3A5 polypeptide or fragment thereof, wherein said polypeptide comprises amino acid substitutions of HGLFK to YGTF. (with the period meaning termination) at a position corresponding to position 30 to 34 of the CYP3A5 polypeptide (Accession No: NP_{—}000768.1, an amino acid substitution of S to Y at a position corresponding to position 100 of the CYP3A5 polypeptide (Accession No: NP_{—}000768.1), an amino acid substitution of R to Q at a position corresponding to position 130 of the CYP3A5 polypeptide (Accession No: NP_{—}000768.1), an amino acid substitution of I to T at a position corresponding to position 149 of the CYP3A5 polypeptide (Accession No: NP_{—}000768.1), an amino acid substitutions of TYD to YL. (with the period meaning termination) at position corresponding to position 346 to 348 of the CYP3A5 polypeptide (Accession No: NP_{—}000768.1), or an amino acid substitution of I to T at a position corresponding to position 488 of the CYP3A5 polypeptide (Accession No: NP_{—}000768.1).

In the context of the present invention the term “polynucleotides” or the term “polypeptides” refers to different variants of a polynucleotide or polypeptide. Said variants comprise a reference or wild type sequence of the polynucleotides or polypeptides of the invention as well as variants which differ therefrom in structure or composition. Reference or wild type sequences for the polynucleotides are Accession No: AF280107.1 and AC005020.2. Reference or wild type sequence for the polypeptides of the invention is Accession No: NP_{—}000768.1. The differences in structure or composition usually occur by way of nucleotide or amino acid substitution(s), addition(s) and/or deletion(s). Preferably, said nucleotide substitution(s), addition(s) or deletion(s) result(s) in one or more changes of the corresponding amino acid(s) of the polypeptides of the invention. The variant polynucleotides and polypeptides also comprise fragments of said polynucleotides or polypeptides of the invention. The polynucleotides and polypeptides as well as the aforementioned fragments thereof of the present invention are characterized as being associated with a CYP3A5 dysfunction or dysregulation. Preferably, said dysfunctions or dysregulations referred to in the present invention cause a disease or disorder or a prevalence for said disease or disorder. Preferably, as will be discussed below in detail, said disease is cancer or diseases including cardiovascular diseases, diabetes and AIDS or any other disease caused by a dysfunction or dysregulation due to a polynucleotide or polypeptides of the invention.

The polynucleotides of the invention include polynucleotides that have at least 70%, preferably at least 75%, at least 80%, at least 85%, at least 90% or at least 95% sequence identity to a CYP3A5 gene, wherein said polynucleotide is having a nucleotide exchange, a nucleotide deletion of at least one nucleotide, or at least one additional nucleotide at a position corresponding to position -20643, -20555, -20359, -20367, -20329, -20323, -20310, -6200, -6177, -4336, -3990, -3868, -3844, -3557, -1617, -795, -86, -74, 136, 174 to 176, 230, 3705, 3709/3710, 5215, 5235, 5516, 7182, 7207, 7303, 7424/7427, 12907, 13028, 13077, 13173, 13226, 13376, 14720, 14836, 14903, 15788, 16079, 16931/16932, 16993, 17163, 19069, 19165, 19208, 27050, 27131/27132, 27526, 31499, 31551 or 31611 of the CYP3A5 gene (Accession No: AF280107.1, wherein position 166220 has been numbered +1 and position 174832 has been numbered +8613, and Accession No: AC005020.2, wherein position 27341 has been numbered +8614).

The term “hybridizing” as used herein refers to polynucleotides which are capable of hybridizing to the polynucleotides of the invention or parts thereof which are associated with a CYP3A5 dysfunction or dysregulation. Thus, said hybridizing polynucleotides are also associated with said dysfunctions and dysregulations. Therefore, said polynucleotides may be useful as probes in Northern or Southern Blot analysis of RNA or DNA preparations, respectively, or can be used as oligonucleotide primers in PCR analysis dependent on their respective size. Also comprised by the invention are hybridizing polynucleotides which are useful for analysing DNA-Protein interactions via, e.g., electrophoretic mobility shift analysis (EMSA). Preferably, said hybridizing polynucleotides comprise at least 10, more preferably at least 15 nucleotides in length while a hybridizing polynucleotide of the present invention to be used as a probe preferably comprises at least 100, more preferably at least 200, or most preferably at least 500 nucleotides in length.

It is well known in the art how to perform hybridization experiments with nucleic acid molecules, i.e. the person skilled in the art knows what hybridization conditions s/he has to use in accordance with the present invention. Such hybridization conditions are referred to in standard text books such as Molecular Cloning A Laboratory Manual, Cold Spring Harbor Laboratory (1989) N.Y. Preferred in accordance with the present inventions are polynucleotides which are capable of hybridizing to the polynucleotides of the invention or parts thereof which are associated with a CYP3A5 dysfunction or dysregulation under stringent hybridization conditions, i.e. which do not cross hybridize to unrelated polynucleotides such as polynucleotides encoding a polypeptide different from the CYP3A5 polypeptides of the invention.

Nucleic acid hybridization will be affected by such conditions as salt concentration, temperature, or organic solvents, in addition to the base composition, length of the complementary strands and the number of nucleotide base mismatches between the hybridizing nucleic acids, as will be readily appreciated by those skilled in the art. Stringent temperature conditions will generally include temeratures in excess of 30° C., typically 37° C., and preferably in excess of 45° C. Stringent salt conditions will ordinarily be less than 1000 mM, typically less than 500 mM and preferably less than 200 mM. However, the combination of parameters is much more important than the measure of any single parameter. See, e.g., Wetmur and Davidson, 1968. Probe sequences may also hybridize specifically to duplex DNA under certain conditions to form triplex or higher order DNA complexes. The preparation of such probes and suitable hybridization conditions are well known in the art.

The term “percent sequence identity” or “identical” in the context of nucleic acid sequences refers to the residues in the two sequences which are the same when aligned for maximum correspondence. The length of sequence identity comparison may be over a strech of at least nine nucleotides, usually at least 20 nucleotides, more usually at least 24 nucleotides, typically at least 28 nucleotides, more typically at least 32 nucleotides, and preferably at least 36 nucleotides or more nucleotides. There are a number of different algorithms known in the art which can be used to measure nucleotide sequence identity. For instance, polynucleotide sequences can be compared using Fasta, a program in GCG Version 6.6. Fasta provides alignments and percent sequence identity of the regions of the best overlap between the query and the search sequence (Pearson, 1980, herein incorporated by reference). For instance, percent sequence identity between nucleic acid sequences can be determined using Fasta with its default parameters (a word size of 6 and the NOPAMfactor for the scoring matrix) as provided in GCG Version 6.1, herein incorporated by reference.

The term “corresponding” as used herein means that a position is not only determined by the number of the preceding nucleotides and amino acids, respectively. The position of a given nucleotide or amino acid in accordance with the present invention which may be deleted, substituted or comprise one or more additional nucleotide(s) may vary due to deletions or additional nucleotides or amino acids elsewhere in the gene or the polypeptide. Thus, under a “corresponding position” in accordance with the present invention it is to be understood that nucleotides or amino acids may differ in the indicated number but may still have similar neighboring nucleotides or amino acids. Said nucleotides or amino acids which may be exchanged, deleted or comprise additional nucleotides or amino acids are also comprised by the term “corresponding position”. Said nucleotides or amino acids may for instance together with their neighbors form sequences which may be involved in the regulation of gene expression, stability of the corresponding RNA or RNA editing, as well as encode functional domains or motifs of the protein of the invention.

By, e.g., “position 3709/3710” it is meant that said polynucleotide comprises one or more additional nucleotide(s) which are inserted between positions 3709 and position 3710 of the corresponding wild type version of said polynucleotide. The same applies mutatis mutandis to all other position numbers referred to in the above embodiment which are drafted in the same format, i.e. two consecutive position numbers separated by a slash (/). By, e.g., “position 7424 to 7427” is meant that said polynucleotide comprises one or more deleted nucleotides which are deleted between positions 7424-and position 7427 of the corresponding wild type version of said polynucleotide and/or one or more additional nucleotide(s) which are inserted between positions 7424 and position 7427 of the corresponding wild type version of said polynucleotide. The same applies mutatis mutandis to all other position numbers referred to in the above embodiment which are drafted in the same format.

The numbering of the polymorphisms refers to the aligned and joined genomic sequences AF280107.1 and AC005020.2, wherein the T at position 174832 (which has been numbered +8613) of the sequence AF280107.1 refers to position 27340 of the sequence AC005020.2. The nucleotide A at position 27341 of the sequence AC005020.2 has been numbered +8614. Numbering of polymorphisms to a position corresponding to a position up to +8613 refers to the genomic sequence AF280107.1, numbering of polymophisms to a position corresponding to position +8614 and greater refer to the genomic sequence AC005020.2.

In accordance with the present invention, the mode and population distribution of genetic variations in the CYP3A5 gene has been analyzed by sequence analysis of relevant regions of the human said gene from many different individuals. It is a well known fact that genomic DNA of individuals, which harbor the individual genetic makeup of all genes, including the CYP3A5 gene, can easily be purified from individual blood samples. These individual DNA samples are then used for the analysis of the sequence composition of the alleles of the CYP3A5 gene that are present in the individual which provided the blood sample. The sequence analysis was carried out by PCR amplification of relevant regions of said genes, subsequent purification of the PCR products, followed by automated DNA sequencing with established methods (e.g. ABI dyeterminator cycle sequencing).

One important parameter that had to be considered in the attempt to determine the individual genotypes and identify novel variants of the CYP3A5 gene by direct DNA-sequencing of PCR-products from human blood genomic DNA is the fact that each human harbors (usually, with very few abnormal exceptions) two gene copies of each autosomal gene (diploidy). Because of that, great care had to be taken in the evaluation of the sequences to be able to identify unambiguously not only homozygous sequence variations but also heterozygous variations. The details of the different steps in the identification and characterization of novel polymorphisms in the CYP3A5 gene (homozygous and heterozygous) are described in the Examples below.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better or may even be highly toxic in some patients than in others and that these variations in patient's responses to drugs can be related to molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252). In this context of population variability with regard to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of patient, for example, and characterization of disease (Bertz, Clin. Pharmacokinet. 32 (1997), 210-256; Engel, J. Chromatogra. B. Biomed. Appl. 678 (1996), 93-103). For the founders of health care, such as health maintenance organizations in the US and government public health services in many European countries, this pharmacogenomics approach can represent a way of both improving health care and reducing overheads because there is a large cost to unnecessary drugs, ineffective drugs and drugs with side effects.

The mutations in the variant genes of the invention sometime result in amino acid deletion(s), insertion(s) and in particular in substitution(s) either alone or in combination. It is of course also possible to genetically engineer such mutations in wild type genes or other mutant forms. Methods for introducing such modifications in the DNA sequence of said genes are well known to the person skilled in the art; see, e.g., Sambrook, Molecular Cloning A Laboratory Manual, Cold Spring Harbor Laboratory (1989) N.Y.

For the investigation of the nature of the alterations in the amino acid sequence of the polypeptides of the invention software programs may be used such as RASMOL that are obtainable from the Internet. Furthermore, folding simulations and computer redesign of structural motifs can be performed using other appropriate computer programs (Olszewski, Proteins 25 (1996), 286-299; Hoffman, Comput. Appl. Biosci. 11 (1995), 675-679). Computers can be used for the conformational and energetic analysis of detailed protein models (Monge, J. Mol. Biol. 247 (1995), 995-1012; Renouf, Adv. Exp. Med. Biol. 376 (1995), 37-45). These analysis can be used for the identification of the influence of a particular mutation on binding and/or processing of drugs.

Usually, said amino acid deletion, addition or substitution in the amino acid sequence of the protein encoded by the polynucleotide of the invention is due to one or more nucleotide substitution, insertion or deletion, or any combinations thereof. Preferably said nucleotide substitution, insertion or deletion may result in amino acid substitutions of HGLFK to YGTF. (with the period meaning termination) at a position corresponding to position 30 to 34 of the CYP3A5 polypeptide (Accession No: NP_{—}000768.1), in an amino acid substitution of S to Y at a position corresponding to position 100 of the CYP3A5 polypeptide (Accession No: NP_{—}000768.1), in amino acid substitutions of TYD to YL. (with the period meaning termination) at a position corresponding to position 346 to 348 of the CYP3A5 polypeptide (Accession No: NP_{—}000768.1), or in an amino acid substitution of T to N at a position corresponding to position 398 of the CYP3A5 polypeptide (Accession No: NP_{—}000768.1).

The mutations in the CYP3A5 gene detected in accordance with the present invention are listed in Table 2A-E. The methods of the mutation analysis followed standard protocols and are described in detail in the Examples. In general such methods are to be used in accordance with the present invention for evaluating the phenotypic spectrum as well as the overlapping clinical characteristics of diseases or conditions related to dysfunctions and diseases related to the drug metabolism. Advantageously, the characterization of said mutants may form the basis of the development of improved drugs, such as drugs which are used e.g. in cancer therapy and diseases including cardiovascular diseases, diabetes and AIDS. Said methods encompass for example haplotype analysis, single-strand conformation polymorphism analysis (SSCA), PCR and direct sequencing, or TaqMan® analysis. On the basis of thorough clinical characterization of many patients the phenotypes can then be correlated to these mutations as well as to mutations that had been described earlier, for example in Jounaidi, Biochem Biophys Res Commun, 221, pp. 466-470, 1996.

Also comprised by the polynucleotides referred to in the present invention are polynucleotides which comprise at least two, preferably at least three, of the polynucleotides specified hereinabove, i.e. polynucleotides having a nucleotide sequence which contains at least two, preferably three of the mutations comprised by the above polynucleotides or listed in the tables below. Thus, the haplotype determined in accordance with the present invention can be characterized by at least two, preferably three of said mutations in the CYP3A5 locus. Further, the polynucleotide of the invention may further comprise at least one-nucleotide deletion, addition and/or substitution other than those specified hereinabove, for example those described in the prior art; e.g., in Jounaidi, Biochem Biophys Res Commun, 221, pp. 466-470, 1996, in Paulussen, Pharmacogenetics 10, pp. 415-424, 2000, in Kuehl, 2001, Nature Genetics 27: 383-391, or in Chou, 2001, Drug Metab Dispos 29: 1205-1209.

This allows the study of synergistic effects of said mutations in the CYP3A5 gene and/or a polypeptide encoded by said polynucleotide on the pharmacological profile of drugs in patients who bear such mutant forms of the gene or similar mutant forms that can be mimicked by the above described proteins. It is expected that the analysis of said synergistic effects provides deeper insights into the onset of dysfunctions or diseases related to drug metabolism as described supra. From said deeper insight the development of diagnostic and pharmaceutical compositions related to dysfunctions or diseases related to drug metabolism will greatly benefit.

Moreover, it has been surprisingly found that the so called positive predictive power for CYP3A5 dysfunctions or dysregulations can be significantly increased based on the polynucleotides of the present invention and thus allows a reliable prediction in contrast to positive predictive power based on the prior art. The increased CYP3A5 protein expression in all except one liver samples (17/18) identified in accordance with the present invention and described in detail in the examples below co-segregates with a haplotype which consists of at least three variants (ch-v-021, ch-v-026, ch-v-015) with distinct locations within or upstream of the gene locus. Genotyping these three variants has in no case led to the generation of false-positive predictions resulting in an estimated positive predictive power for the 3-variant genotype of about 99.95%. This is in striking contrast to the positive predictive power determined for the haplotype described by Paulussen, Pharmacogenetics 10, pp. 415-424, 2000 which is about 65%. Moreover, based on the polynucleotides of the invention and as described in the examples below, it has been found that the SNPs described by Paulussen, Pharmacogenetics 10, pp. 415-424, 2000 are located in contrary to what is reported in said document approximately 20 kb upstream of the transcriptional start site of the CYP3A5 gene in a sequence 5′ to a CYP3A5 pseudogene locus.

Therefore, the haplotypes characterized on the basis of the polynucleotides of the present invention fulfil the criteria expected from a reliable marker of CYP3A5 expression. As is evident to the person skilled in the art, the genetic knowledge deduced from the present invention can now be used to exactly and reliably characterize the genotype of a patient. Advantageously, diseases or a prevalence for a disease which are associated with CYP3A5 dysfunction or dysregulation, such as cancer, diseases including cardiovascular diseases, diabetes and AIDS, can be predicted and preventive or therapeutical measures can be applied accordingly. Moreover in accordance with the foregoing, in cases where a given drug takes an unusual effect, a suitable individual therapy can be designed based on the knowledge of the individual genetic makeup of a subject with respect to the polynucleotides of the invention and improved therapeutics can be developed as will be further discussed below.

Finally, the polynucleotides and polypeptides referred to in accordance with the present invention are also useful as forensic markers, which improve the identification of subjects which have been murdered or killed by, for example, a crime of violence or any other violence and can not be identified by the well known conventional forensic methods. The application of forensic methods based on the detection of the polymorphisms comprised by the polynucleotides of this invention in the genome of a subject are particularly well suited in cases where a (dead) body is disfigured in a severe manner such that identification by other body characteristics such as the features of the face is not possible. This is the case, for example, for corpse found in water which are usually entirely disfigured. Advantageously methods which are based on the provision of the polynucleotides of the invention merely require a minimal amount of tissues or cells in order to be carried out. Said tissues or cells may be blood droplets, hair roots, epidermal scales, saliva droplets, sperms etc. Since only such a minimal amount of tissues or cells are required for the identification of a subject, the polymorphisms comprised by the polynucleotides of this invention can be also used as forensic markers in order to prove someone guilt of a crime, such as a violation or a ravishment. Moreover, the polymorphisms comprised by the polynucleotides of this invention can be used to proof paternity. In accordance with the forensic methods referred to herein the presence or absence of the polynucleotides of the invention is determined and compared with a reference sample which is unambiguously derived from the subject to be identified. The forensic methods which require detection of the presence or absence of the polynucleotides of the invention in a sample of a subject the polymorphisms comprised by the polynucleotides of this invention can be for example PCR-based techniques which are particularly well suited in cases where only a minimal amount of tissues or cells are available as forensic samples. On the other hand, where enough tissue or cells are available, hybridization based techniques may be performed in order to detect the presence or absence of a polynucleotide of this invention. These techniques are well known by the person skilled in the art and can be adopted to the individual purposes referred to herein without further ado. In conclusion, thanks to the present invention forensic means which allow improved and reliable predictions as regards the aforementioned aspects are now available.

In line with the foregoing, preferably, the polynucleotide of the present invention is associated with cancer or diseases including cardiovascular diseases, diabetes and AIDS.

The term “cancer” used herein is very well known and characterized in the art. Several variants of cancer exist and are comprised by said term as meant in accordance with the invention. For a detailed list of symptoms which are indicative for cancer it is referred to text book knowledge, e.g. Pschyrembel.

In a further embodiment the present invention relates to a polynucleotide which is DNA or RNA.

The polynucleotide of the invention may be, e.g., DNA, cDNA, genomic DNA, RNA or synthetically produced DNA or RNA or a recombinantly produced chimeric nucleic acid molecule comprising any of those polynucleotides either alone or in combination. Preferably said polynucleotide is part of a vector, particularly plasmids, cosmids, viruses and bacteriophages used conventionally in genetic engineering that comprise a polynucleotide of the invention. Such vectors may comprise further genes such as marker genes which allow for the selection of said vector in a suitable host cell and under suitable conditions.

The invention furthermore relates to a gene comprising the polynucleotide of the invention.

It is well known in the art that genes comprise structural elements which encode an amino acid sequence as well as regulatory elements which are involved in the regulation of the expression of said genes. Structural elements are represented by exons which may either encode an amino acid sequence or which may encode for RNA which is not encoding an amino acid sequence but is nevertheless involved in RNA function, e.g. by regulating the stability of the RNA or the nuclear export of the RNA.

Regulatory elements of a gene may comprise promoter elements or enhancer elements both of which could be involved in transcriptional control of gene expression. It is very well known in the art that a promoter is to be found upstream of the structural elements of a gene. Regulatory elements such as enhancer elements, however, may be found distributed over the entire locus of the gene. Said elements could be reside, e.g., in introns, regions of genomic DNA which separate the exons of a gene. Said introns may comprise further regulatory elements which are required for proper gene expression. Introns are usually transcribed together with the exons of a gene resulting in a nascent RNA transcript which contains both, exon and intron sequences. The intron encoded RNA sequences are usually removed by a process known as RNA splicing. However, said process also requires regulatory sequences present on a RNA transcript, said regulatory sequences may be encoded by the introns.

In addition, besides their function in transcriptional control and control of proper RNA processing and/or stability, regulatory elements of a gene could be also involved in the control of genetic stability of a gene locus. Said elements control, e.g., recombination events or serve to maintain a certain structure of the DNA or the arrangement of DNA in a chromosome.

Therefore, polymorphisms can occur in exons of-a gene which encode an amino acid sequence as discussed supra as well as in regulatory regions which are involved in the above discussed process. The analysis of the nucleotide sequence of a gene locus in its entirety including, e.g., introns is in light of the above desirable. It has been found based on the polymorphisms comprised by the polynucleotides of the present invention that the mechanism of the increased expression of CYP3A5 protein in most Caucasians livers described in the examples below may involve enhanced transcription and stabilisation of the gene's transcripts.

Therefore, in a furthermore preferred embodiment of the gene of the invention a nucleotide deletion, addition and/or substitution results in altered expression of the variant gene compared to the corresponding wild type gene.

In another embodiment the present invention relates to a vector comprising the polynucleotide of the invention or the gene of the invention.

Said vector may be, for example, a phage, plasmid, viral or retroviral vector. Retroviral vectors may be replication competent or replication defective. In the latter case, viral propagation generally will occur only in complementing host/cells.

The polynucleotides or genes of the invention may be joined to a vector containing selectable markers for propagation in a host. Generally, a plasmid vector is introduced in a precipitate such as a calcium phosphate precipitate, or in a complex with a charged lipid or in carbon-based clusters. Should the vector be a virus, it may be packaged in vitro using an appropriate packaging cell line prior to application to host cells.

In a more preferred embodiment of the vector of the invention the polynucleotide is operatively linked to expression control sequences allowing expression in prokaryotic or eukaryotic cells or isolated fractions thereof.

Expression of said polynucleotide comprises transcription of the polynucleotide, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells.

The term “isolated fractions thereof” refers to fractions of eukaryotic or prokaryotic cells or tissues comprising said cells which are capable of transcribing or transcribing and translating RNA from the vector of the invention. Said fractions comprise proteins which are required for transcrition of RNA or transcription of RNA and translation of said RNA into a polypeptide. Said isolated fractions may be, e.g., nuclear and cytoplasmic fractions of eukaryotic cells such as of reticulocytes.

The present invention furthermore relates to a host cell genetically engineered with the polynucleotide of the invention, the gene of the invention or the vector of the invention.

Said host cell may be a prokaryotic or eukaryotic cell; see supra. The polynucleotide or vector of the invention which is present in the host cell may either be integrated into the genome of the host cell or it may be maintained extrachromosomally. In this respect, it is also to be understood that the recombinant DNA molecule of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press.

The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a polynucleotide for the expression of a variant polypeptide of the invention. Prokaryotic hosts may include gram negative as well as gram positive bacteria such as, for example, E. coli, S. typhimurium, Serratia marcescens and Bacillus subtilis. A polynucleotide coding for a mutant form of variant polypeptides of the invention can be used to transform or transfect the host using any of the techniques commonly known to those of ordinary skill in the art.

Methods for preparing fused, operably linked genes and expressing them in bacteria or animal cells are well-known in the art (Sambrook, supra). The genetic constructs and methods described therein can be utilized for expression of variant polypeptides of the invention in, e.g., prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed prokaryotic hosts can be grown in fermentors and cultured according to techniques known in the art to achieve optimal cell growth. The proteins of the invention can then be isolated from the grown medium, cellular lysates, or cellular membrane fractions. The isolation and purification of the microbially or otherwise expressed polypeptides of the invention may be by any conventional means such as, for example, preparative chromatographic separations and immunological separations such as those involving the use of monoclonal or polyclonal antibodies.

Thus, in a further embodiment the invention relates to a method for producing a molecular variant polypeptide or fragment thereof comprising culturing the above described host cell; and recovering said protein or fragment from the culture.

In another embodiment the present invention relates to a method for producing cells capable of expressing a molecular variant polypeptide comprising genetically engineering cells with the polynucleotide of the invention, the gene of the invention or the vector of the invention.

The cells obtainable by the method of the invention can be used, for example, to test drugs according to the methods described in D. L. Spector, R. D. Goldman, L. A. Leinwand, Cells, a Lab manual, CSH Press 1998. Furthermore, the cells can be used to study known drugs and unknown derivatives thereof for their ability to complement the deficiency caused by mutations in the CYP3A5 gene. For these embodiments the host cells preferably lack a wild type allele, preferably both alleles of the CYP3A5 gene and/or have at least one mutated from thereof. Ideally, the gene comprising an allele as comprised by the polynucleotides of the invention could be introduced into the wild type locus by homologous replacement. Alternatively, strong overexpression of a mutated allele over the normal allele and comparison with a recombinant cell line overexpressing the normal allele at a similar level may be used as a screening, and analysis system. The cells obtainable by the above-described method may also be used for the screening methods referred to herein below.

Furthermore, the invention relates to a polypeptide or fragment thereof encoded by the polynucleotide of the Invention, the gene of the invention or obtainable by the method described above or from cells produced by the method described above.

In this context it is also understood that the variant polypeptide of the invention can be further modified by conventional methods known in the art. By providing said variant proteins according to the present invention it is also possible to determine the portions relevant for their biological activity or inhibition of the same. The terms “polypeptide” and “protein” as used herein are exchangeable. Moreover, what is comprised by said terms is standard textbook knowledge.

The present invention furthermore relates to an antibody which binds specifically to the polypeptide of the invention.

Advantageously, the antibody specifically recognizes or binds an epitope containing one or more amino acid substitution(s) as defined above. Antibodies against the variant polypeptides of the invention can be prepared by well known methods using a purified protein according to the invention or a (synthetic) fragment derived therefrom as an antigen. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals. In a preferred embodiment of the invention, said antibody is a monoclonal antibody, a polyclonal antibody, a single chain antibody, human or humanized antibody, primatized, chimerized or fragment thereof that specifically binds said peptide or polypeptide also including bispecific antibody, synthetic antibody, antibody fragment, such as Fab, Fv or scFv fragments etc., or a chemically modified derivative of any of these. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the variant polypeptides of the invention as well as for the monitoring of the presence of said variant polypeptides, for example, in recombinant organisms, and for the identification of compounds interacting with the proteins according to the invention. For example, surface plasmon resonance as employed in the BIAcore system can be used to increase the efficiency of phage antibodies which bind to an epitope of the protein of the invention (Schier, Human Antibodies Hybridomas 7 (1996), 97-105; Malmborg, J. Immunol. Methods 183 (1995), 7-13).

In a preferred embodiment the antibody of the present invention specifically recognizes an epitope containing one or more amino acid substitution(s) resulting from a nucleotide exchange as defined supra.

Antibodies which specifically recognize modified amino acids such as phospho-Tyrosine residues are well known in the art. Similarly, in accordance with the present invention antibodies which specifically recognize even a single amino acid exchange in an epitope may be generated by the well known methods described supra.

In light of the foregoing, in a more preferred embodiment the antibody of the present invention is monoclonal or polyclonal.

The invention also relates to a transgenic non-human animal comprising at least one polynucleotide of the invention, the gene of the invention or the vector of the invention as described supra.

The present invention also encompasses a method for the production of a transgenic non-human animal comprising introduction of a polynucleotide or vector of the invention into a germ cell, an embryonic cell, stem cell or an egg or a cell derived therefrom. The non-human animal can be used in accordance with the method of the invention described below and may be a non-transgenic healthy animal, or may have a disease or disorder, preferably a disease caused by at least one mutation in the gene of the invention. Such transgenic animals are well suited for, e.g., pharmacological studies of drugs in connection with variant forms of the above described variant polypeptides since these polypeptides or at least their functional domains are conserved between species in higher eukaryotes, particularly in mammals. Production of transgenic embryos and screening of those can be performed, e.g., as described by A. L. Joyner Ed., Gene Targeting, A Practical Approach (1993), Oxford University Press. The DNA of the embryos can be analyzed using, e.g., Southern blots with an appropriate probe or based on PCR techniques.

A transgenic non-human animal in accordance with the invention may be a transgenic mouse, rat, hamster, dog, monkey, rabbit, pig, frog, nematode such as Caenorhabditis elegans, fruitfly such as Drosophila melanogaster or fish such as torpedo fish or zebrafish comprising a polynucleotide or vector of the invention or obtained by the method described above, preferably wherein said polynucleotide or vector is stably integrated into the genome of said non-human animal, preferably such that the presence of said polynucleotide or vector leads to the expression of the variant polypeptide of the invention. It may comprise one or several copies of the same or different polynucleotides or genes of the invention. This animal has numerous utilities, including as a research model for cancer or diseases including cardiovascular diseases, diabetes and AIDS or any other disease caused by as dysfunction or dysregulation of the polynucleotides or polypeptides of the invention research and therefore, presents a novel and valuable animal in the development of therapies, treatment, etc. for cancer diseases or diseases including cardiovascular diseases, diabetes and AIDS or any other disease caused by as dysfunction or dysregulation of the polynucleotides or polypeptides of the invention. Accordingly, in this instance, the mammal is preferably a laboratory animal such as a mouse or rat.

Thus, in a preferred embodiment the transgenic non-human animal of the invention is a mouse, a rat or a zebrafish.

Numerous reports revealed that said animals are particularly well suited as model organisms for the investigation of the drug metabolism and its deficiencies or cancer. Advantageously, transgenic animals can be easily created using said model organisms, due to the availability of various suitable techniques well known in the art.

The invention also relates to a solid support comprising one or a plurality of the polynucleotide, the gene, the vector, the polypeptide, the antibody or the host cell of the invention in immobilized form.

The term “solid support” as used herein refers to a flexible or non-flexible support that is suitable for carrying said immobilized targets. Said solid support may be homogenous or inhomogeneous. For example, said solid support may consist of different materials having the same or different properties with respect to flexibility and immobilization, for instance, or said solid support may consist of one material exhibiting a plurality of properties also comprising flexibility and immobilization properties. Said solid support may comprise glass-, polypropylene- or silicon-chips, membranes, oligonucleotide-conjugated beads or bead arrays.

The term “immobilized” means that the molecular species of interest is fixed to a solid support, preferably covalently linked thereto. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physico-chemical interactions typically occur in interactions between molecules. For example, biotinylated polypeptides may be fixed on a avidin-coated solid support due to interactions of the above described types. Further, polypeptides such as antibodies, may be fixed on an antibody coated solid support. Moreover, the immobilization is dependent on the chemical properties of the solid support. For example, the nucleic acid molecules can be immobilized on a membrane by standard techniques such as UV-crosslinking or heat.

In a preferred embodiment of the invention said solid support is a membrane, a glass- or poylpropylene- or silicon-chip, are oligonucleotide-conjugated beads or a bead array, which is assembled on an optical filter substrate.

Moreover, the present invention relates to an in vitro method for identifying a polymorphism said method comprising the steps of:
(a) isolating a polynucleotide or the gene of the invention from a plurality of subgroups of individuals, wherein one subgroup has no prevalence for a CYP3A5 associated disease and at least one or more further subgroup(s) do have prevalence for a CYP3A5 associated disease; and
(b) identifying a polymorphism by comparing the nucleic acid sequence of said polynucleotide or said gene of said one subgroup having no prevalence for a CYP3A5 associated disease with said at least one or more further subgroup(s) having a prevalence for a CYP3A5 associated disease.

The term “prevalence” as used herein means that individuals are susceptible for one or more disease(s) which are associated with CYP3A5 dysfuntion or dysregulation or could already have one or more of said disease(s). Thereby, one CYP3A5 associated disease can be used to determine the susceptibility for another CYP3A5 associated disease, e.g. impaired drug metabolism may be indicative for a prevalence for, e.g. cancer. Moreover, symptoms which are indicative for a prevalence for developing said diseases are very well known in the art and have been sufficiently described in standard textbooks such as Pschyrembel.

Advantageously, polymorphisms according to the present invention which are associated with CYP3A5 dysfunction or dysregulation or one or more disease(s) based thereon should be enriched in subgroups of individuals which have a prevalence for said diseases versus subgroups which have no prevalence for said diseases. Thus, the above described method allows the rapid and reliable detection of polymorphisms which are indicative for one or more CYP3A5 associated disease(s) or a susceptibility therefor. Advantageously, due to the phenotypic preselection a large number of individuals having no prevalence might be screened for polymorphisms in general. Thereby, a reference sequences comprising polymorphisms which do not correlate to one or more CYP3A5 associated disease(s) can be obtained. Based on said reference sequences it is possible to efficiently and reliably determine the relevant polymorphisms.

In a further embodiment the present invention relates to a method for identifying and obtaining a pro-drug or a drug capable of modulating the activity of a molecular variant of a CYP3A5 polypeptide comprising the steps of:
(a) contacting the polypeptide, the solid support of the invention, a cell expressing a molecular variant gene comprising a polynucleotide of the invention, the gene or the vector of the invention in the presence of components capable of providing a detectable signal in response to drug activity with a compound to be screened for pro-drug or drug activity; and
(b) detecting the presence or absence of a signal or increase or decrease of a signal generated from the pro-drug or the drug activity, wherein the absence, presence, increase or decrease of the signal is indicative for a putative pro-drug or drug.

The term “compound” in a method of the invention includes a single substance or a plurality of substances which may or may not be identical.

Said compound(s) may be chemically synthesized or produced via microbial fermentation but can also be comprised in, for example, samples, e.g., cell extracts from, e.g., plants, animals or microorganisms. Furthermore, said compounds may be known in the art but hitherto not known to be useful as an inhibitor, respectively. The plurality of compounds may be, e.g., added to the culture medium or injected into a cell or non-human animal of the invention.

If a sample containing (a) compound(s) is identified in the method of the invention, then it is either possible to isolate the compound from the original sample identified as containing the compound in question or one can further subdivide the original sample, for example, if it consists of a plurality of different compounds, so as to reduce the number of different substances per sample and repeat the method with the subdivisions of the original sample. It can then be determined whether said sample or compound displays the desired properties, for example, by the methods described herein or in the literature (Spector et al., Cells manual; see supra). Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art or by using and modifying the methods as described herein. Furthermore, the person skilled in the art will readily recognize which further compounds may be used in order to perform the methods of the invention, for example, enzymes, if necessary, that convert a certain compound into a precursor. Such adaptation of the method of the invention is well within the skill of the person skilled in the art and can be performed without undue experimentation.

Compounds which can be used in accordance with the present invention include peptides, proteins, nucleic acids, antibodies, small organic compounds, ligands, peptidomimetics, PNAs and the like. Said compounds may act as agonists or antagonists of the invention. Said compounds can also be functional derivatives or analogues of known drugs. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, N.Y., USA. Furthermore, said derivatives and analogues can be tested for their effects according to methods known in the art or as described. Furthermore, peptide mimetics and/or computer aided design of appropriate drug derivatives and analogues can be used, for example, according to the methods described below. Such analogs comprise molecules may have as the basis structure of known CYP3A5 substrates and/or inhibitors and/or modulators; see infra.

Appropriate computer programs can be used for the identification of interactive sites of a putative inhibitor and the polypeptides of the invention by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used in combination with the method of the invention for, e.g., optimizing known inhibitors, analogs, antagonists or agonists. Appropriate peptidomimetics and other inhibitors can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of said compounds and the polypeptides of the invention can be used for the design of peptidomimetic drugs (Rose, Biochemistry 35 (1996), 12933-12944; Rutenber, Bioorg. Med. Chem. 4 (1996), 1545-1558). It is very well known how to obtain said compounds, e.g. by chemical or biochemical standard techniques. Thus, also comprised by the method of the invention are means of making or producing said compounds. In summary, the present invention provides methods for identifying and obtaining compounds which can be used in specific doses for the treatment of specific forms of CYP3A5 associated diseases, e.g. dysfunctions of the drug metabolism or cancer.

The above definitions apply mutatis mutandis to all of the methods described in the following.

In a further embodiment the present invention relates to a method for identifying and obtaining an inhibitor of the activity of a molecular variant of a CYP3A5 polypeptide comprising the steps of:
(a) contacting the protein, the solid support of the invention or a cell expressing a molecular variant gene comprising a polynucleotide or the gene or the vector of the invention in the presence of components capable of providing a detectable signal in response to drug activity with a compound to be screened for inhibiting activity; and
(b) detecting the presence or absence of a signal or increase or decrease of a signal generated from the inhibiting activity, wherein the absence or decrease of the signal is indicative for a putative inhibitor.

In a preferred embodiment of the method of the invention said cell is a cell, obtained by the method of the invention or can be obtained from the transgenic non-human animal as described supra.

In a still further embodiment the present invention relates to a method of identifying and obtaining a pro-drug or drug capable of modulating the activity of a molecular variant of a CYP3A5 polypeptide comprising the steps of:
(a) contacting the host cell, the cell obtained by the method of the invention, the polypeptide or the solid support of the invention with the first molecule known to be bound by a CYP3A5 polypeptide to form a first complex of said polypeptide and said first molecule;
(b) contacting said first complex with a compound to be screened, and
(c) measuring whether said compound displaces said first molecule from said first complex.

Advantageously, in said method said measuring step comprises measuring the formation of a second complex of said protein and said inhibitor candidate. Preferably, said measuring step comprises measuring the amount of said first molecule that is not bound to said protein.

In a particularly preferred embodiment of the above-described method of said first molecule is a agonist or antagonist or a substrate and/or a inhibitor and/or a modulator of the polypeptide of the invention, e.g., with a radioactive or fluorescent label.

In a still another embodiment the present invention relates to a method of identifying and obtaining an inhibitor capable of modulating the activity of a molecular variant of a CYP3A5 polypeptide comprising the steps of:
(a) contacting the host cell or the cell obtained by the method of the invention, the protein or the solid support of the invention with the first molecule known to be bound by a CYP3A5 polypeptide to form a first complex of said protein and said first molecule;
(b) contacting said first complex with a compound to be screened, and
(c) measuring whether said compound displaces said first molecule from said first complex.

In a preferred embodiment of the method of the invention said measuring step comprises measuring the formation of a second complex of said protein and said compound.

In another preferred embodiment of the method of the invention said measuring step comprises measuring the amount of said first molecule that is not bound to said protein.

In a more preferred embodiment of the method of the invention said first molecule is labeled.

The invention furthermore relates to a method for the production of a pharmaceutical composition comprising the steps of the method as described supra; and the further step of formulating the compound identified and obtained or a derivative thereof in a pharmaceutically acceptable form.

The therapeutically useful compounds identified according to the methods of the invention can be formulated and administered to a patient as discussed above. For uses and therapeutic doses determined to be appropriate by one skilled in the art and for definitions of the term “pharmaceutical composition” see infra.

Furthermore, the present invention encompasses a method for the preparation of a pharmaceutical composition comprising the steps of the above-described methods; and formulating a drug or pro-drug in the form suitable for therapeutic application and preventing or ameliorating the disorder of the subject diagnosed in the method of the invention.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or inhibitor identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329).

In a preferred embodiment of the method of the present invention said drug or prodrug is a derivative of a medicament as defined hereinafter.

The present invention also relates to a method of diagnosing a disorder related to the presence of a molecular variant of the CYP3A5 gene or susceptibility to such a disorder comprising determining the presence of a polynucleotide or the gene of the invention in a sample from a subject.

In accordance with this embodiment of the present invention, the method of testing the status of a disorder or susceptibility to such a disorder can be effected by using a polynucleotide gene or nucleic acid of the invention, e.g., in the form of a Southern or Northern blot or in situ analysis. Said nucleic acid sequence may hybridize to a coding region of either of the genes or to a non-coding region, e.g. intron. In the case that a complementary sequence is employed in the method of the invention, said nucleic acid molecule can again be used in Northern blots. Additionally, said testing can be done in conjunction with an actual blocking, e.g., of the transcription of the gene and thus is expected to have therapeutic relevance. Furthermore, a primer or oligonucleotide can also be used for hybridizing to one of the above mentioned CYP3A5 gene or corresponding mRNAs. The nucleic acids used for hybridization can, of course, be conveniently labeled by incorporating or attaching, e.g., a radioactive or other marker. Such markers are well known in the art. The labeling of said nucleic acid molecules can be effected by conventional methods.

Additionally, the presence or expression of variant CYP3A5 gene can be monitored by using a primer pair that specifically hybridizes to either of the corresponding nucleic acid sequences and by carrying out a PCR reaction according to standard procedures. Specific hybridization of the above mentioned probes or primers preferably occurs at stringent hybridization conditions. The term “stringent hybridization conditions” is well known in the art; see, for example, Sambrook et al., “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985. Furthermore, the mRNA, cRNA, cDNA or genomic DNA obtained from the subject may be sequenced to identify mutations which may be characteristic fingerprints of mutations in the polynucleotide or the gene of the invention. The present invention further comprises methods wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, RNase T₂ or the like or a ribozyme and, for example, electrophoretically separating and detecting the RNA fragments as described above.

Further modifications of the above-mentioned embodiment of the invention can be easily devised by the person skilled in the art, without any undue experimentation from this disclosure; see, e.g., the examples. An additional embodiment of the present invention relates to a method wherein said determination is effected by employing an antibody of the invention or fragment thereof. The antibody used in the method of the invention may be labeled with detectable tags such as a histidine flags or a biotin molecule.

The invention relates to a method of diagnosing a disorder related to the presence of a molecular variant of a CYP3A5 gene or susceptibility to such a disorder comprising determining the presence of a polypeptide or the antibody of the invention in a sample from a subject.

In a preferred embodiment of the above described method said disorder is cancer or diseases including cardiovascular diseases, diabetes and AIDS.

In a preferred embodiment of the present invention, the above described method is comprising PCR, ligase chain reaction, restriction digestion, direct sequencing, nucleic acid amplification techniques, hybridization techniques or immunoassays. Said techniques are very well known in the art.

Moreover, the invention relates to a method of detection of the polynucleotide or the gene of the invention in a sample comprising the steps of
(a) contacting the solid support described supra with the sample under conditions allowing interaction of the polynucleotide or the gene of the invention with the immobilized targets on a solid support and;
(b) determining the binding of said polynucleotide or said gene to said immobilized targets on a solid support.

The invention also relates to an in vitro method for diagnosing a disease comprising the steps of the method described supra, wherein binding of said polynucleotide or gene to said immobilized targets on said solid support is indicative for the presence or the absence of said disease or a prevalence for said disease.

The invention furthermore relates to a diagnostic composition comprising the polynucleotide, the gene, the vector, the polypeptide or the antibody of the invention.

In addition, the invention relates to a pharmaceutical composition comprising the polynucleotide, the gene, the vector, the polypeptide or the antibody of the invention.

These pharmaceutical compositions comprising, e.g., the antibody may conveniently be administered by any of the routes conventionally used for drug administration, for instance, orally, topically, parenterally or by inhalation. Acceptable salts comprise acetate, methylester, HCl, sulfate, chloride and the like. The compounds may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

Furthermore, the use of pharmaceutical compositions which comprise antisense-oligonucleotides which specifically hybridize to RNA encoding mutated versions of the polynucleotitde or gene according to the invention or which comprise antibodies specifically recognizing a mutated polypeptide of the invention but not or not substantially the functional wild-type form is conceivable in cases in which the concentration of the mutated form in the cells should be reduced.

Thanks to the present invention the particular drug selection, dosage regimen and corresponding patients to be treated can be determined in accordance with the present invention. The dosing recommendations will be indicated in product labeling by allowing the prescriber to anticipate dose adjustments depending on the considered patient group, with information that avoids prescribing the wrong drug to the wrong patients at the wrong dose.

In another embodiment the present invention relates to the use of the polynucleotide, a polynucleotide comprising SEQ ID No: 104, a polynucleotide encoding a polypeptide comprising SEQ ID No: 145, the gene, the vector or the polypeptide of the invention, a polypeptide comprising SEQ ID No: 145 or the antibody of the invention for the preparation of a diagnostic composition for diagnosing a disease.

A gene encoding a functional and expressible polypeptide of the invention can be introduced into the cells which in turn produce the protein of interest. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The gene may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell. As is evident from the above, it is preferred that in the use of the invention the nucleic acid sequence is operatively linked to regulatory elements allowing for the expression and/or targeting of the polypeptides of the invention to specific cells. Suitable gene delivery systems that can be employed in accordance with the invention may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Standard methods for transfecting cells with recombinant DNA are well known to those skilled in the art of molecular biology, see, e.g., WO 94/29469; see also supra. Gene therapy may be carried out by directly administering the recombinant DNA molecule or vector of the invention to a patient or by transfecting cells with the polynucleotide or vector of the invention ex vivo and infusing the transfected cells into the patient.

A polynucleotide comprising SEQ ID No: 104 and a polypeptide comprising SEQ ID No: 145 have already been described in Jounaidi et al. (Jounaidi, Biochem Biophys Res Commun 221 (1996), 466-70). However, Jounaidi et al. have merely disclosed the respective amino acid and nucleotide sequences without making any suggestion towards the pharmaceutic and diagnostic value of said polynucleotide or polypeptide, in particular for those disorders and diseases referred to infra.

In a further embodiment the present invention relates to the use of the polynucleotide, a polynucleotide comprising SEQ ID No: 104, a polynucleotide encoding a polypeptide comprising SEQ ID No: 145, the gene, the vector, the polypeptide of the invention, a polypeptide comprising SEQ ID No.: 145 or the antibody of the invention for the preparation of a pharmaceutical composition for treating a disease.

In another embodiment the present invention encompasses the use of a polynucleotide selected from the group consisting of:
(a) a polynucleotide having the nucleic acid sequence of SEQ ID NO: 082, 088, 104, 112, 126, 131, or 140;
(b) a polynucleotide encoding a polypeptide having the amino acid sequence of SEQ ID No: 127, 132, 141 or 145;
(c) a polynucleotide capable of hybridizing to a CYP3A5 gene, wherein said polynucleotide is having at least one additional nucleotide at a position corresponding to.position 3709/3710 or 27131/27132 of the CYP3A5 gene (Accession No: AF280107.1, wherein position 166220 has been numbered +1 and position 174832 has been numbered +8613, and Accession No:

AC005020.2, wherein position 27341 has been numbered +8614) or a nucleotide exchange at a position corresponding to position 7303 or 27289 of the CYP3A5 gene (Accession No: AF280107.1, wherein position 166220 has been numbered +1 and position 174832 has been numbered +8613, and Accession No: AC005020.2, wherein position 27341 has been numbered +8614);
(d) a polynucleotide capable of hybridizing to a CYP3A5 gene, wherein said polynucleotide is having an additional G nucleotide at a position corresponding to position 3709/3710 of the CYP3A5 gene (Accession No:

AF280107.1, wherein position 166220 has been numbered +1 and position 174832 has been numbered +8613, and Accession No: AC005020.2, wherein position 27341 has been numbered +8614), an additional T nucleotide at a position corresponding to position 27131/27132 of the CYP3A5 gene (Accession No: AF280107.1, wherein position 166220 has been numbered +1 and position 174832 has been numbered +8613, and Accession No: AC005020.2, wherein position 27341 has been numbered +8614), or an A at a position corresponding to position 7303 or 27289 of the CYP3A5 gene (Accession No: AF280107.1, wherein position 166220 has been numbered +1 and position 174832 has been numbered +8613, and Accession No: AC005020.2, wherein position 27341 has been numbered +8614);

for the preparation of a diagnostic composition for diagnosing a disease in a subject having a genome comprising a variant allele of the CYP3A5 gene, wherein said allele is having an A at a position corresponding to position 6986 of the CYP3A5 gene (Accession No: AF280107.1, wherein position 166220 has been numbered +1 and position 174832 has been numbered +8613, and Accession No: AC005020.2, wherein position 27341 has been numbered +8614).

The definitions of the terms referred to in this specification apply mutatis mutandis to the aforementioned use.

The term “subject” inter alia refers to animals. Preferably, said animals belong to the animal species referred to above. Moreover, the term “subject” encompasses humans. The humans in accordance with the use of the present invention are selected from all existing ethnical groups and subgroups, e.g. Caucasians, African Americans or Asians. However, particular well suited for the use of the invention are African Americans for which it could be demonstrated that diagnosing a disease or a prevalence for a disease based on monitoring the presence or absence of an CYP3A5 allele having at a position corresponding to position 6986 of the CYP3A5 gene (Accession No: AF280107.1, wherein position 166220 has been numbered +1 and position 174832 has been numbered +8613, and Accession No: AC005020.2, wherein position 27341 has been numbered +8614) an A results in a false positive prediction for CYP3A5 expression in a considerable number of subjects. This allele of CYP3A5 has been described in detail in Kuehl, 2001, Nature Genetics 27: 383-391 as CYP3A5*i allele. The CYP3A5*1 allele is characterized by the presence of an A at position 22893 of the CYP3A5 nucleic acid sequence referred to in Kuehl, loc.cit. The allelic frequency of said allele is particularly high in African Americans although it is also present in other ethnical groups or subgroups. However, it was found in accordance with the present invention that the CYP3A5 expression of subjects for which a false positive result was obtained in diagnostic studies based on the CYP3A5*1 allele could be correctly predicted by further diagnosing the presence or absence of a polynucleotide as defined under (a) to (d) in accordance with the use of the present invention. For example, a polynucleotide having an additional nucleotide at a position corresponding to position 27131/27132 of the CYP3A5 gene as defined supra has been found in accordance with this invention to be present in approximately 10% of the African Americans resulting in a frameshift mutation in exon 11. The present invention provides means and methods to distinguish between the haplotype resulting in improved expression of CYP3A5 comprising the polymorphism(s) of the CYP3A5*1 allele and the haplotype resulting in decreased expression, wherein said haplotype as set forth above comprises in addition to the polymorphism(s) of the CYP3A5*1 allele co-segregating polymorphisms comprised by a polynucleotide referred to under (a) to (d) supra. Thus, based on the aforementioned use of the present invention a reliable diagnosis of the CYP3A5 activity of a subject is achieved.

The invention also relates to the use of a polynucleotide comprising a polynucleotide having an A at a position corresponding to position 14690 of the CYP3A5 gene (Accession No: AF280107.1, wherein position 166220 has been numbered +1 and position 174832 has been numbered +8613, and Accession No: AC005020.2, wherein position 27341 has been numbered +8614) for the preparation of a diagnostic composition for diagnosing a disease in a subject having a genome comprising a variant allele of the CYP3A5 gene, wherein said allele is having an A at a position corresponding to position 6986 of the CYP3A5 gene (Accession No: AF280107.1, wherein position 166220 has been numbered +1 and position 174832 has been numbered +8613, and Accession No: AC005020.2, wherein position 27341 has been numbered +8614).

The definitions of the terms referred to in this specification apply mutatis mutandis to the aforementioned use.

In accordance with the present invention it could also be shown that the polymorphism(s) constituting the CYP3A5*1 and the CYP3A5*6 allele as described in Kuehl, loc.cit., co-segregate in a considerable number of subjects and thereby constitute another haplotype resulting in decreased CYP3A5 expression. It has been shown that the CYP3A5*6 allele results in inappropriate splicing of exon 7 of CYP3A5, a frameshift and a premature termination at position 184 of the CYP3A5 protein. Consequently, false positive result as regards the expression level of CYP3A5 in a subject can be obtained in diagnostic studies based on the CYP3A5*1 allele. Said false positive results, however, can be avoided according to the use of this invention by further diagnosing the presence or absence of the polymorphism(s) of the CYP3A5*6 allele. Thus, based on the aforementioned use of the present invention a reliable diagnosis of the CYP3A5 activity of a subject is achieved.

In light with the foregoing, in a preferred embodiment of the aforementioned use said subject is an African American.

As has been discussed above, the number of subjects which are diagnosed false positive is due to the high allelic frequency of CYP3A5 alleles such as those comprising a polynucleotide as defined under (a) to (d) above resulting in a frame shift mutation or those comprised by the CYP3A5*6 allele. Said allelic frequency is particularly high within the group of African Americans. In accordance with the present invention it has been found that the CYP3A5*6 allele is present in about 13.3% of the African Americans. Thus within this ethnic group the problems emerging from a wrong prediction of CYP3A5 expression are more severe than for other ethnical groups.

In a more preferred embodiment of the use of the present invention said disease is cancer or diseases including cardiovascular diseases, diabetes and AIDS.

Finally, the present invention relates to a diagnostic kit for detection of a polymorphism comprising the polynucleotide, the gene, the vector, the polypeptide, the antibody, the host cell, the transgenic non-human animal or the solid support of the invention.

The kit of the invention may contain further ingredients such as selection markers and components for selective media suitable for the generation of transgenic cells and animals. The kit of the invention can be used for carrying out a method of the invention and could be, inter alia, employed in a variety of applications, e.g., in the diagnostic field or as research tool. The parts of the kit of the invention can be packaged individually in vials or in combination in suitable containers or multicontainer units. Manufacture of the kit follows preferably standard procedures which are known to the person skilled in the art. The kit may be used for methods for detecting expression of a mutant form of the polypeptides, genes or polynucleotides in accordance with any one of the above-described methods of the invention, employing, for example, immunoassay techniques such as radioimmunoassay or enzymeimmunoassay or preferably nucleic acid hybridization and/or amplification techniques such as those described herein before and in the Examples as well as pharmacokinetic studies when using non-human transgenic animals of the invention.

The figures illustrate the invention.

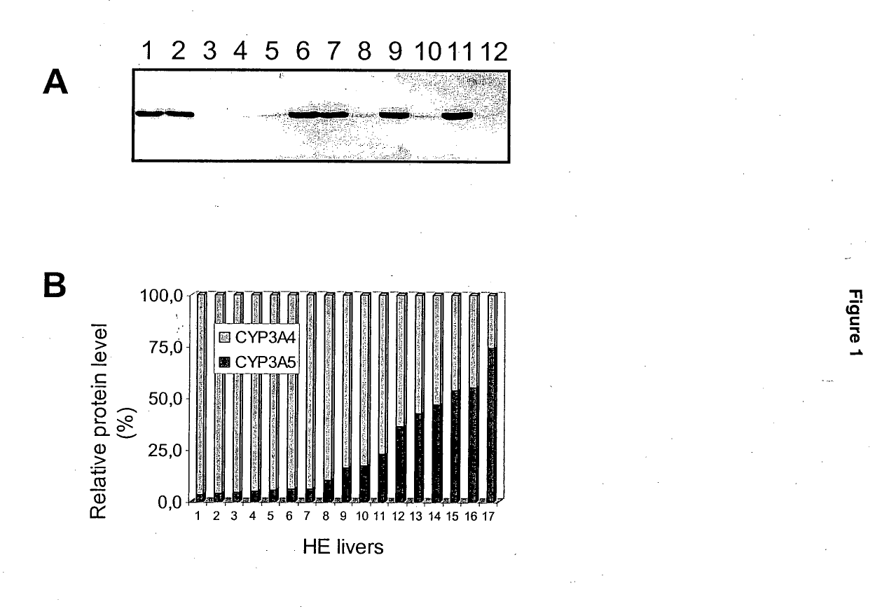

FIG. 1: A. Western blot analysis of CYP3A5 protein expression in microsomes prepared from 6 LE (low expressing) and 6 HE (high expressing) Caucasian livers. B. The relative contributions of CYP3A5 and CYP3A4 to the combined CYP3A5/CYP3A4 protein pool in 17 HE livers as determined by Western blot.

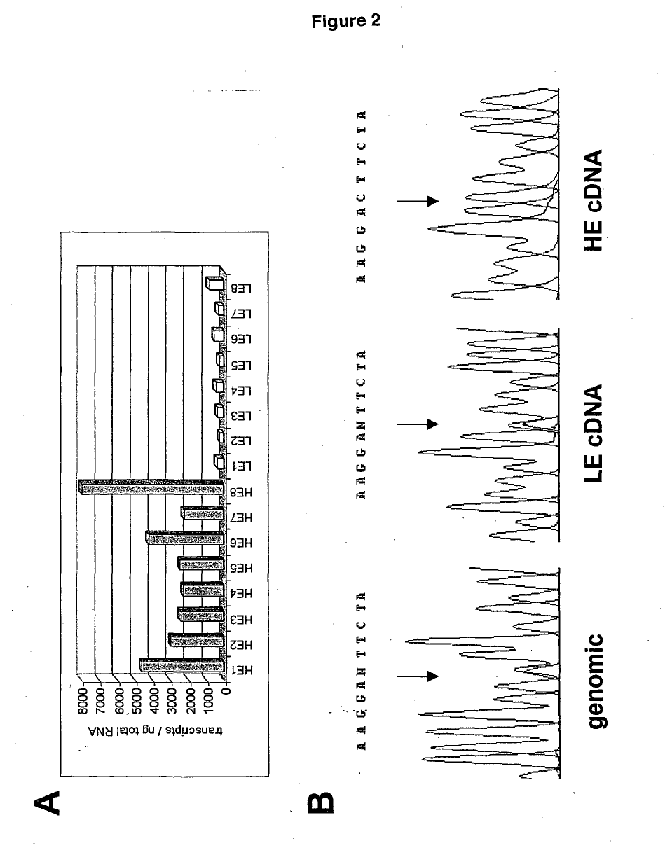

FIG. 2: Expression levels and the allelic source of CYP3A5 transcripts in LE and HE Caucasian livers. A. TaqMan analysis of CYP3A5 mRNA in 8 LE (low expressing, white bars) and 8 HE (high expressing, grey bars) liver samples. B. Sequences of a portion of the 3′-UTR of CYP3A5 in samples heterozygous for variant ch-v-015 (Table 2A). Templates used for PCR were genomic DNA (left panel), cDNA from a LE liver (middle panel)-and cDNA-from an HE liver (right panel).

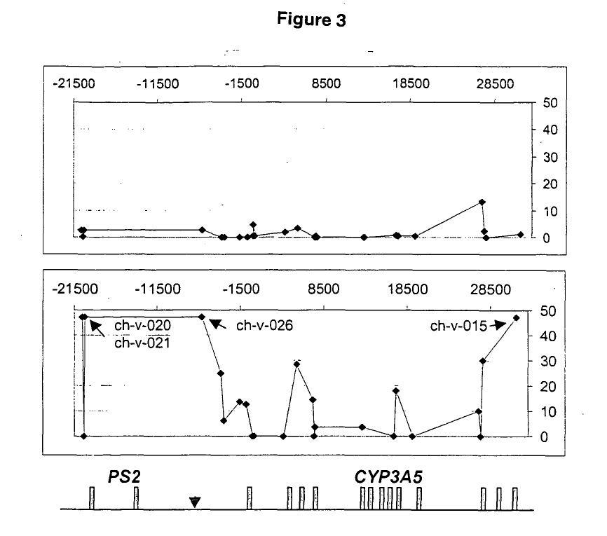

FIG. 3: Allelic frequencies of CYP3A5 genetic variants in Caucasians. A. in DNA samples derived from 8-168 LE individuals B. in DNA samples derived from 7-18 HE individuals. The bars at the bottom of the figure indicate schematically the localisation of the pseudoexons PS2 exon 1 and 2 and exons 1-13 of the CYP3A5 gene. The arrowhead marks the duplication boundary (Gellner, Pharmacogenetics 11 (2001), 111-121).

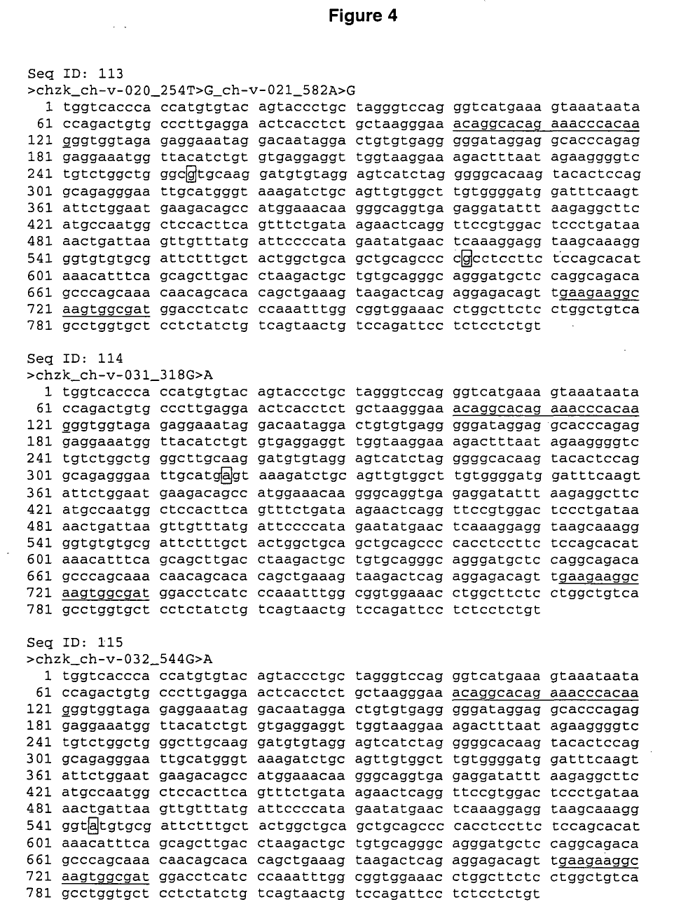

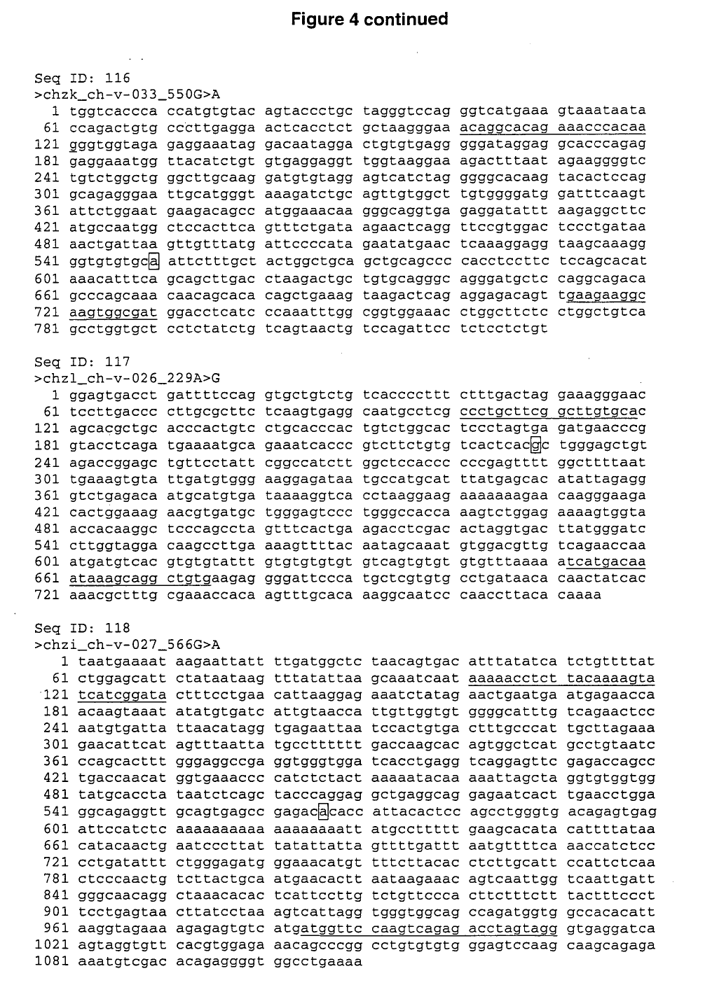

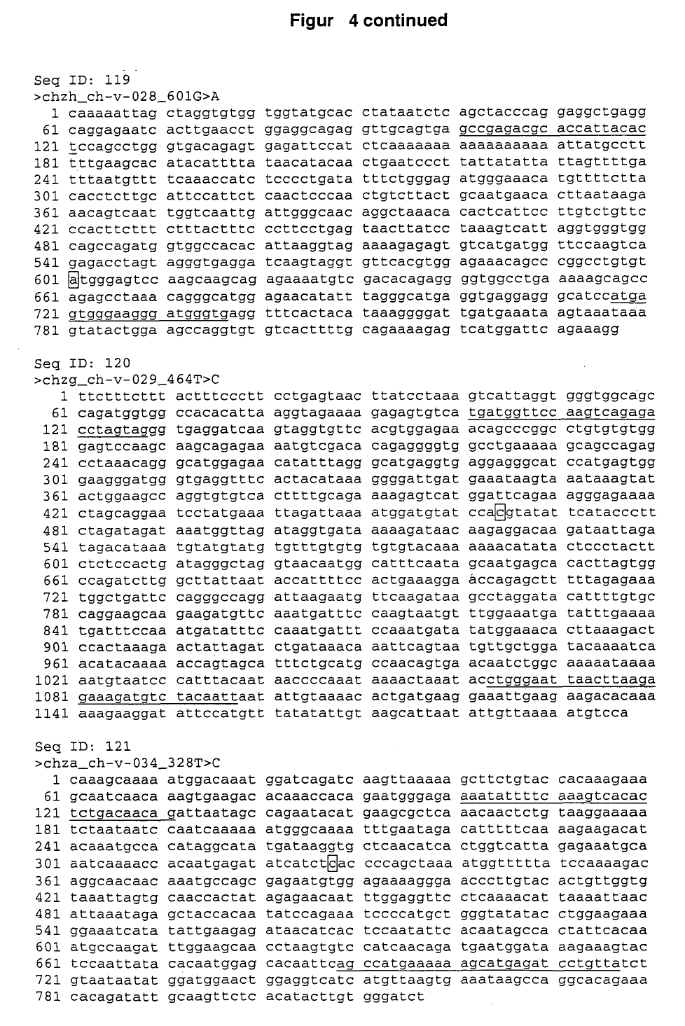

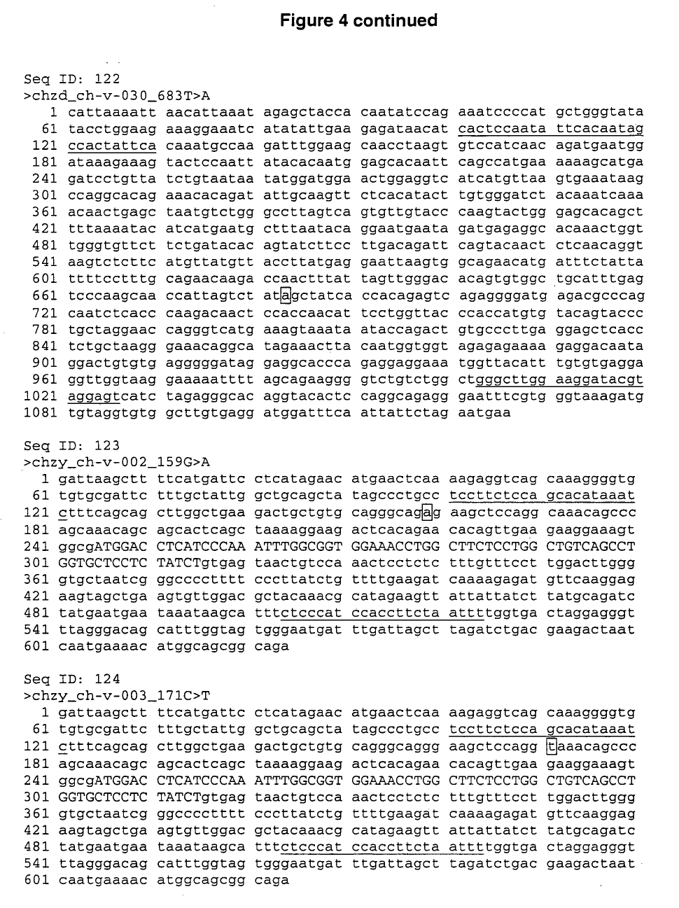

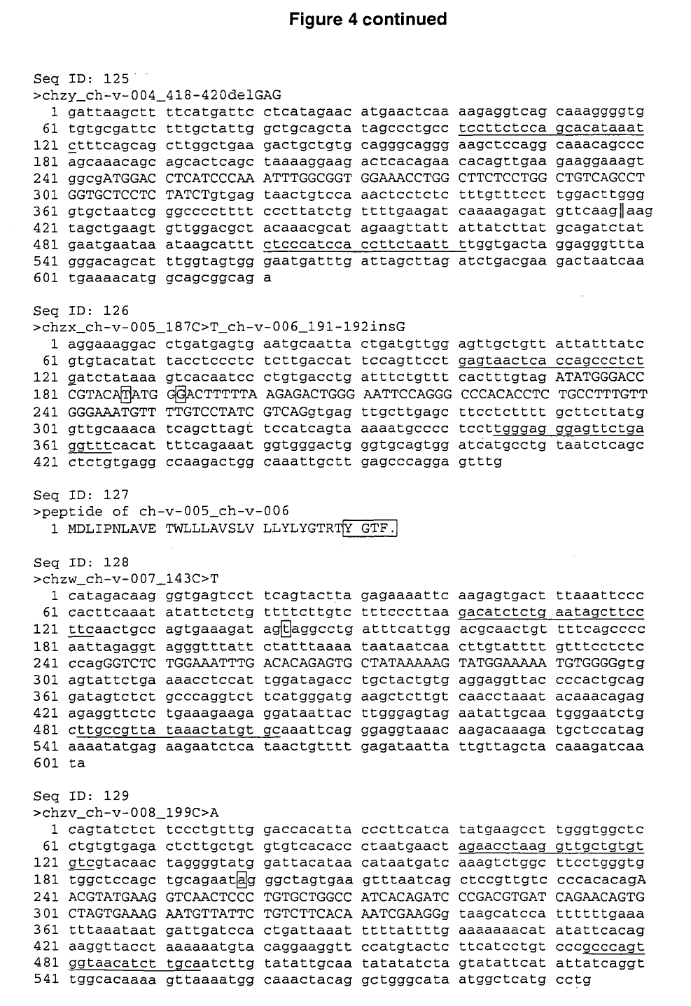

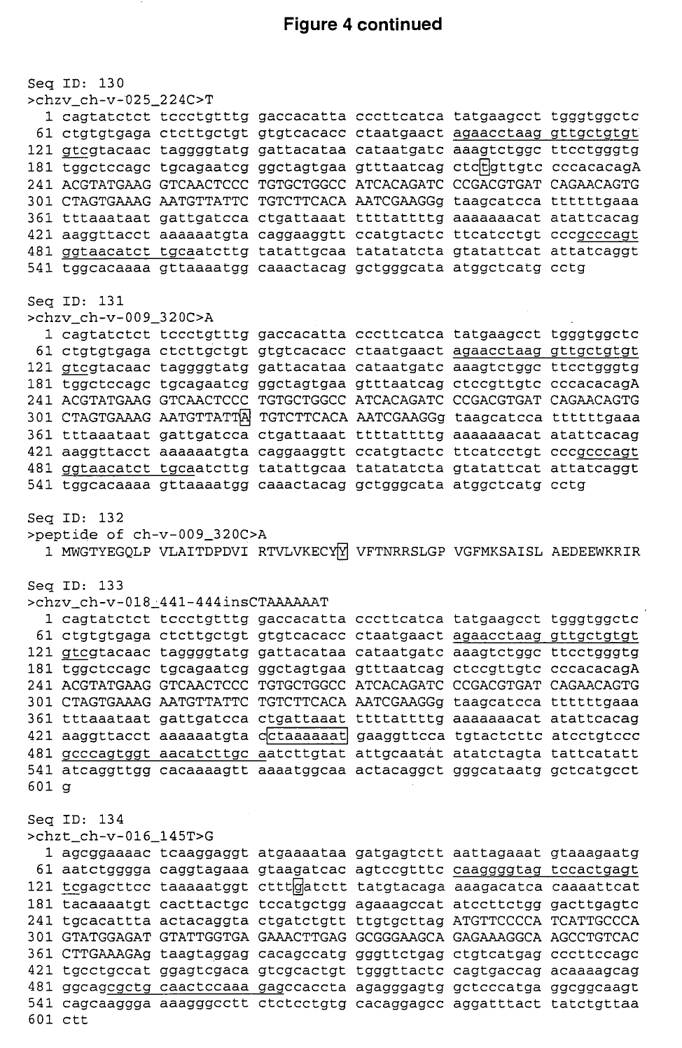

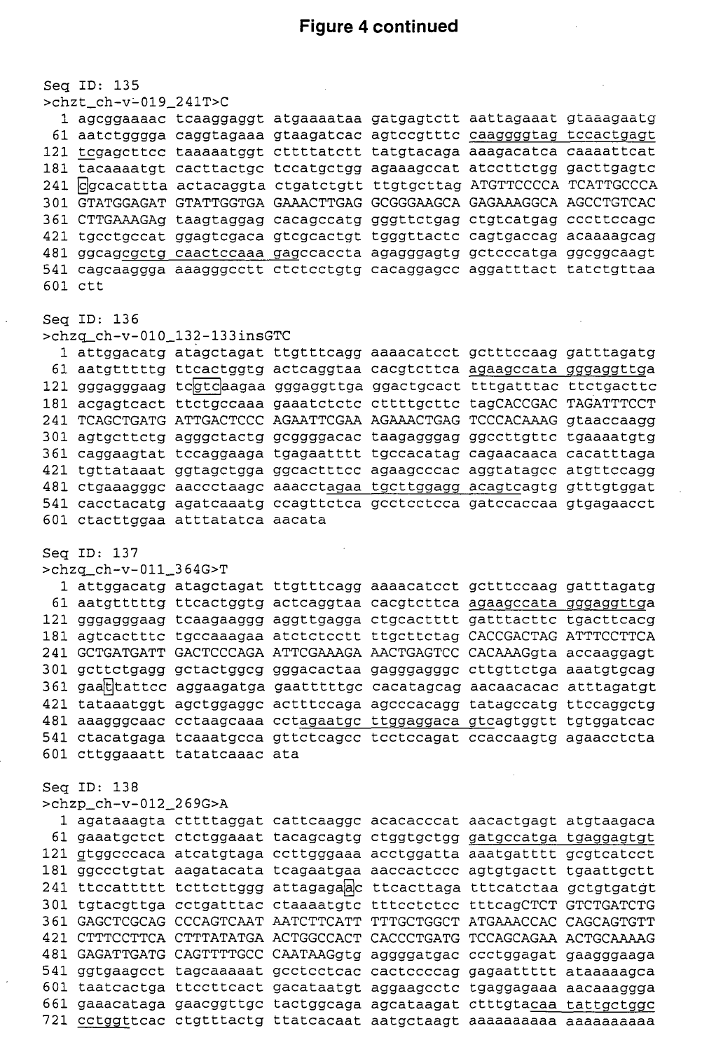

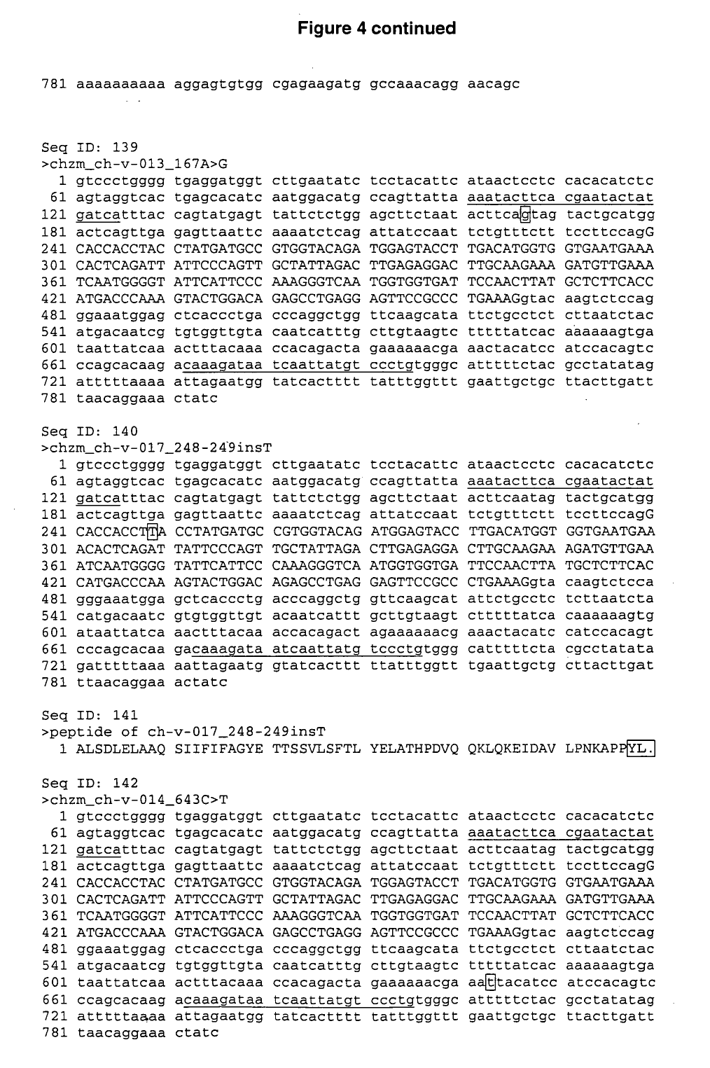

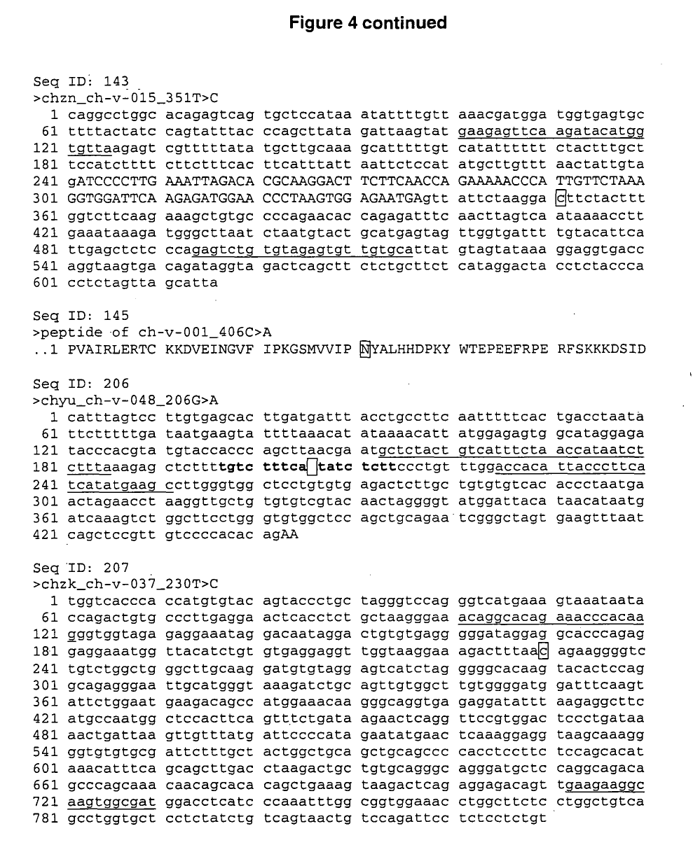

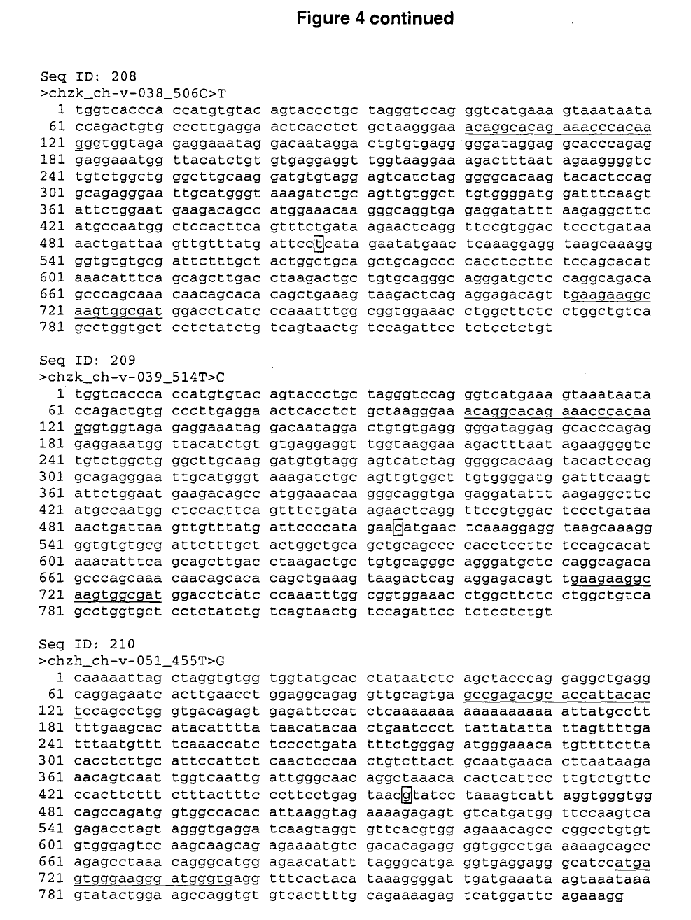

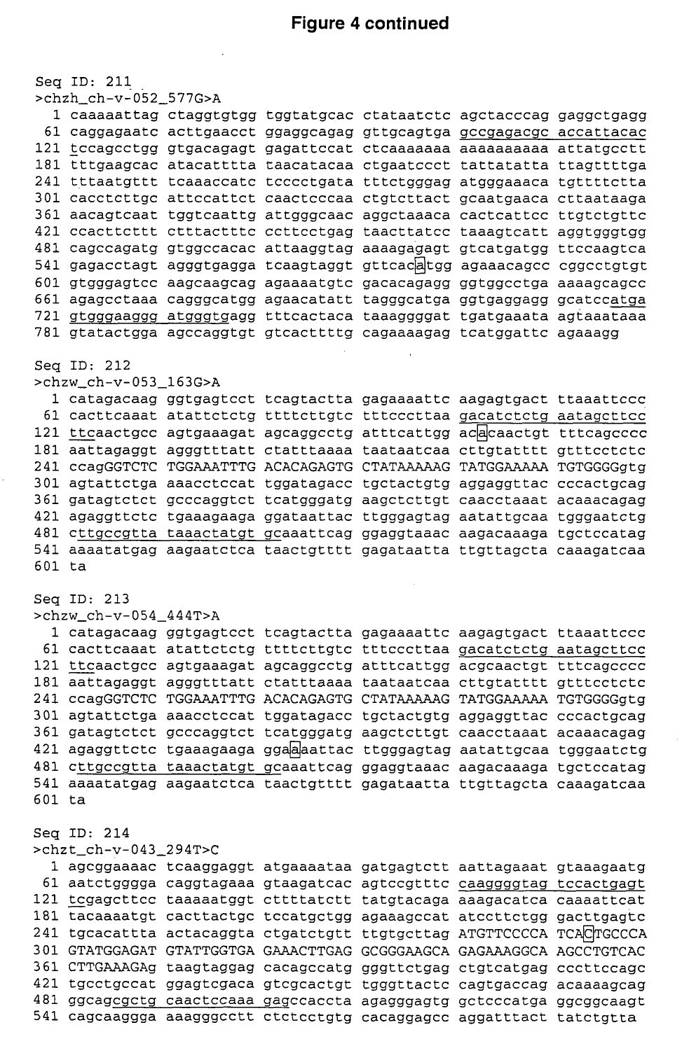

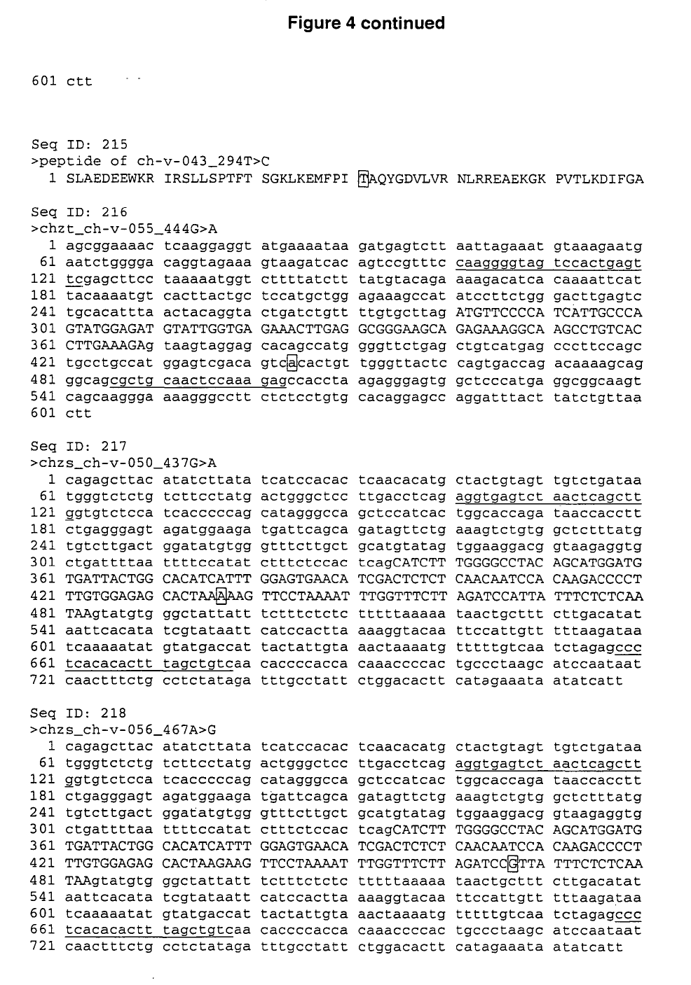

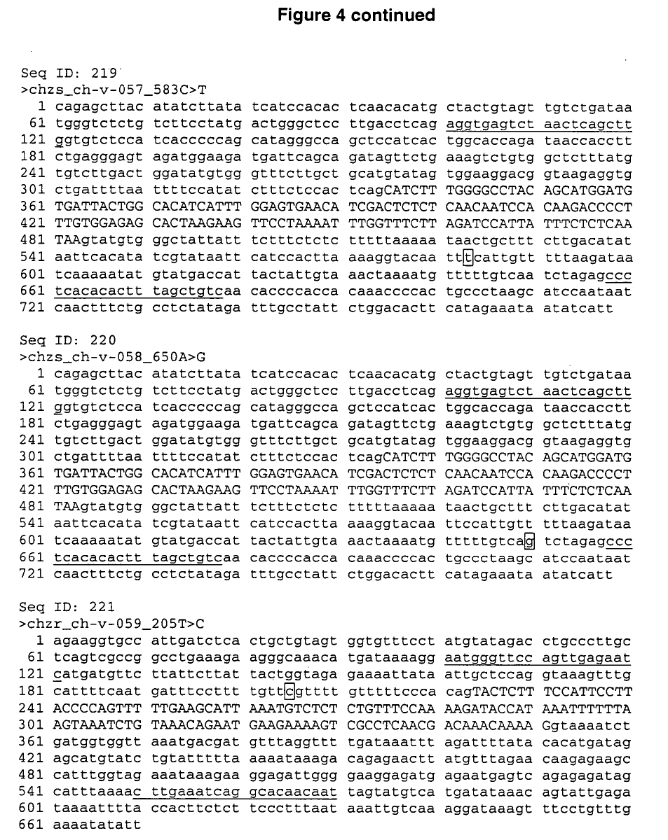

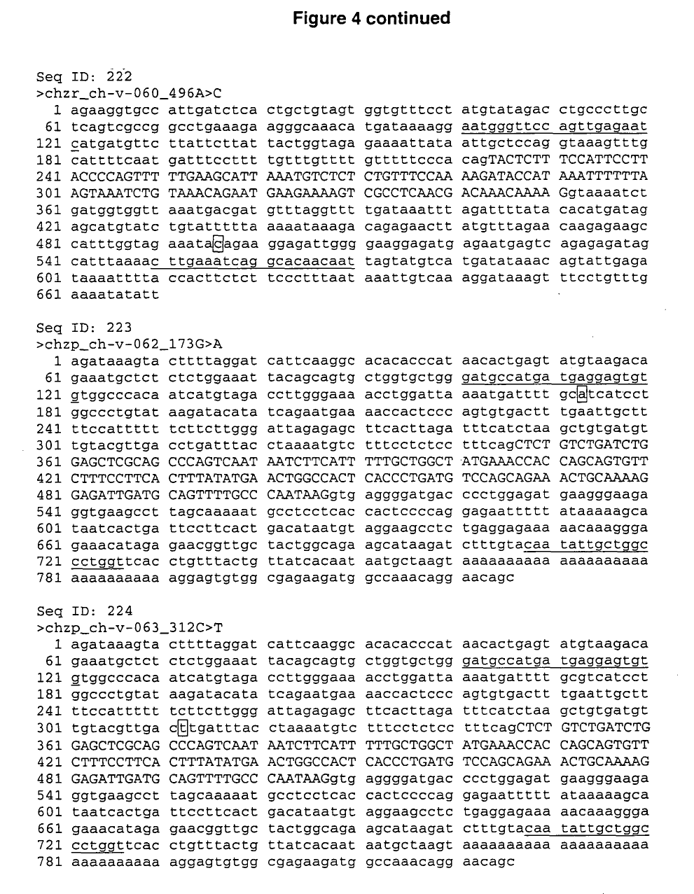

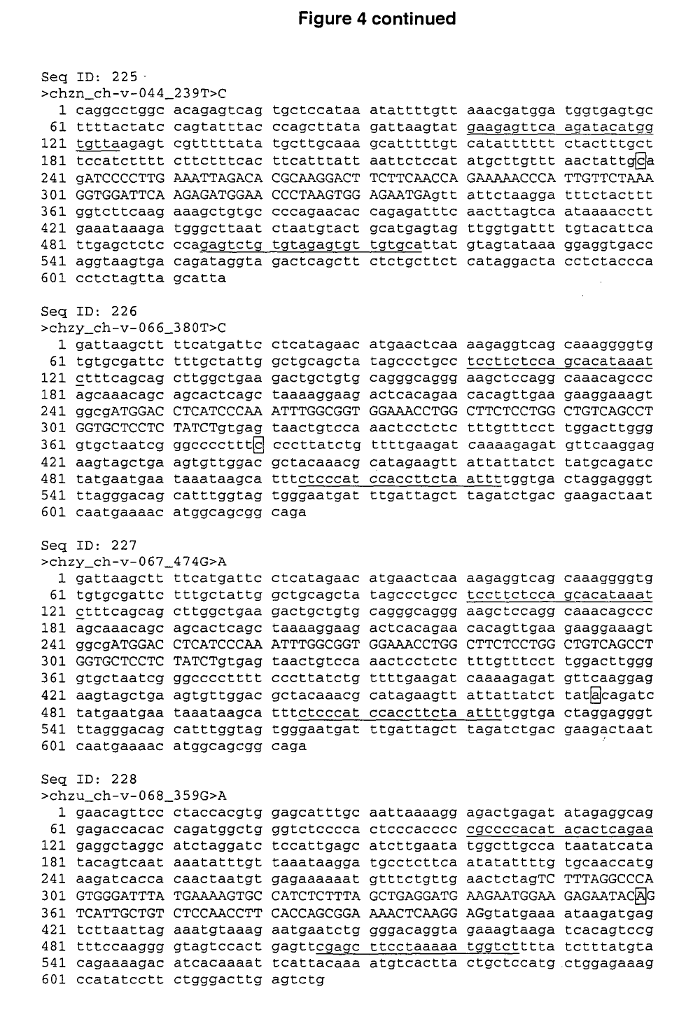

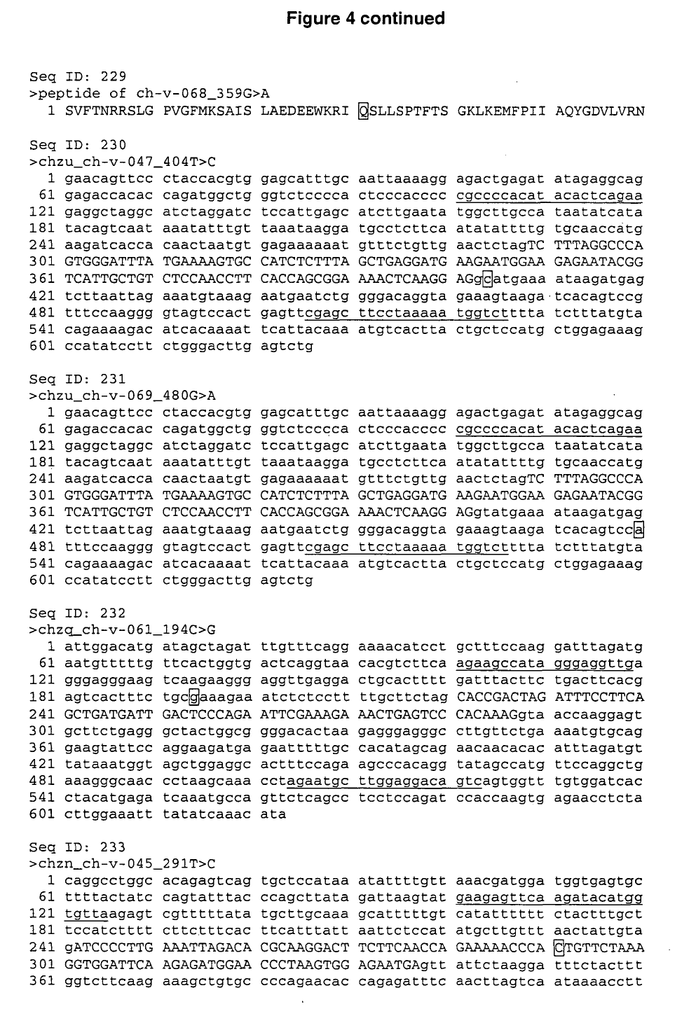

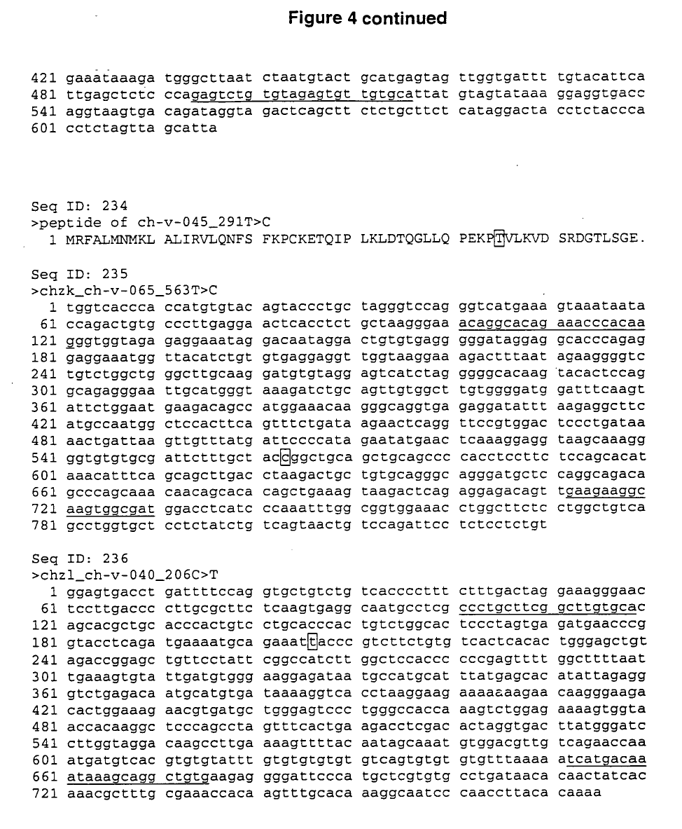

FIG. 4: Genomic and peptide sequences: genomic DNA sequences containing the amplified regions in which polymorphisms were detected and polypeptide sequences with amino acid substitutions. Nucleotide sequences are listed in 5′-3′ orientation. Letters in lowercase indicate non-coding sequences, letters in uppercase indicate coding sequences. Primer regions are underlined. Variant sites are shown framed. Peptide sequences are shown in one letter code. ∥ marks a site where a deletion has occurred. In Seq ID 198, the hybridzing site of the TaqMan® probe is shown in bold.

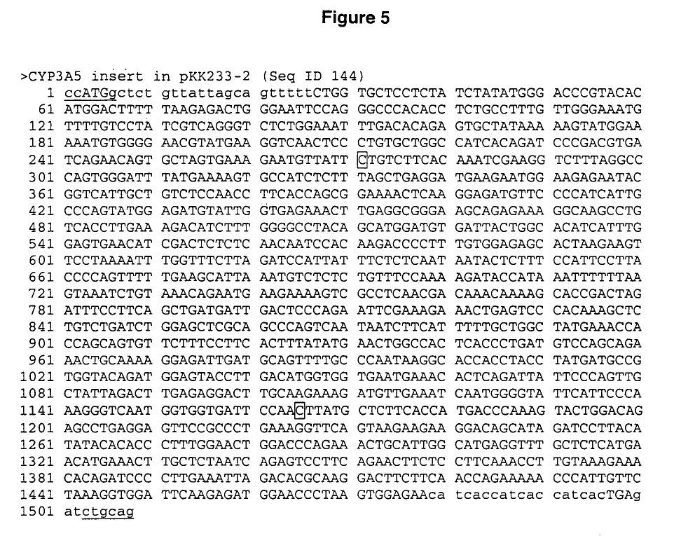

FIG. 5: CYP3A5 cDNA insert region of the plasmid that was used as starting material for in vitro mutagenesis. Cloning sites are shown underlined. Modified 5′ and 3′ regions of the CYP3A5 cDNA are shown in lowercase letters. The 5′ modification, a MALLLAVF amino acid sequence on protein level, has been introduced in order to increase expression in E. coli (Gillam, Arch Biochem Biophys 317 (1995), 374-84). The 3′ modification, a His₆ tag on protein level, has been introduced in order to enhance subsequent purification. The unmodified part of the CYP3A5 insert was verified to be identical to the CYP3A5 cDNA corresponding to accession no. NM_{—}000777.1 by sequencing, which is the underlying nucleotide sequence for NP_{—}000768.1. Sites corresponding to ch-v-009 and ch-v-001 are shown framed.

The invention will now be described by reference to the following biological Examples which are merely illustrative and are not constructed as a limitation of the scope of the present invention.

### EXAMPLE 1

### Isolation of Genomic DNA from Human Blood, Generation and Purification of CYP3A5 Gene Fragments

Genomic DNA was isolated from blood or liver samples using Qiagen blood and tissue DNA isolation kits. Oligonucleotides used in the screen were designed based on the recently determined sequence and organisation of the human CYP3A locus (Gellner, Pharmacogenetics 11 (2001), 111-121). Primer sequences and PCR fragment lengths are given in Table 1A. Amplified fragments were processed through PCR purification columns (Qiagen) and sequenced on PE ABI 3700 DNA Analysers using the same primers as in PCR. The sequences were analysed for the presence of polymorphisms using the PHRED/PHRAP/POLYPHRED/CONSED software package (University of Washington, Seattle, Wash., USA).

Total RNA was isolated from liver samples using the RNeasy kit (Qiagen) according to the manufacturers instructions except that an additional DNase I digestion was performed directly on the column. cDNA pools were generated from 1 μg of total RNA using random hexamer primers and Superscript reverse transcriptase (Life Technologies). The cDNA used for one TaqMan assay was derived from 40 ng total RNA. CYP3A5 mRNA expression levels were quantified by real time quantitative PCR using the ABI 7700 Sequence Detection System (PE Biosystems). Oligonucleotides and probes were designed with the Primer Express (PE Biosystems) programme. Oligonucleotides used for the quantitative PCR were: forward 5′-TTG TTG GGA AAT GTT TTG TCC TAT C-3′ (Seq ID: 237) and reverse 5′-ACA GGG AGT TGA CCT TCA TAC GTT-3′ (Seq ID: 238). The TaqMan probe (5′-TCA GGG TCT CTG GAA ATT TGA CAC AGA GTG CTA-3′; Seq ID: 239) was labelled with the 5′ reporter dye 6-carboxy-fluorescein (FAM) and the 3′ quencher 6-carboxy-tetramethylrhodamin (TAMRA). The experiments were performed according to a standard protocol developed by PE Biosystems. The specificity of the assay for CYP3A5 was determined using equal amounts of CYP3A4, CYP3A5, CYP3A7 and CYP3A43 cDNA species expressed in vitro. The specificity of the probe was 10⁴ times higher for CYP3A5 than for CYP3A7 cDNA whereas CYP3A4 and CYP3A43 cDNAs were not detectable at all. The linear range of the CYP3A5 assay was determined to be between 10 and 10⁶ target molecules. CYP3A5 expression levels were normalised using the expression of 18S mRNA species determined with pre-developed TaqMan assays (PE Biosystems).

### EXAMPLE 2

### Determination of Genetic Variations within the CYP3A5 Locus

Sequence diversity within the CYP3A5 locus was determined by PCR amplification from genomic DNA (fragment size: 264-997 bp) and sequencing each PCR-product of 19-217 samples of Caucasian origin, 36-45 samples of African American origin, 34-47 samples of Chinese origin, 41-50 samples of Japanese origin, and 31-47 samples of Korean origin. The PCR fragments encompass the entire protein-coding region of CYP3A5, a portion of the 3′-UTR, the entire 5′-UTR as well as 6203 bp sequence between the CYP3A5 transcriptional start site and a L1_{—}5′UTR_ORF repeat located upstream of the gene (FIG. 3). In addition, we genotyped two linked single nucleotide polymorphisms (SNPs, ch-v-020, ch-v-021, Table 2A-E) located in a sequence originally described as CYP3A5 promoter that were recently reported to co-segregate with increased CYP3A5 protein expression (Paulussen, Pharmacogenetics 10 (2000), 415-24). The results also indicate co-segregation of both variants. Using the recently determined sequence of the entire CYP3A locus (Gellner, Pharmacogenetics 11 (2001), 111-121), we place these variants approximately 20 kb upstream of the first exon of CYP3A5, in a sequence 5′ adjacent to a CYP3A pseudogene (PS2 in FIG. 3). Furthermore, we additionally genotyped a single nucleotide polymorphism located in intron 3 of the CYP3A5 gene (ch-v-048; Kuehl, 2001, Nature Genetics 27: 383-391) by TaqMan® assay using the primers and probes listed in Table 1B. The analysis were performed on a Sequence Detection System (PE Biosystems).

A total of 29 variants including the two linked SNPs described by Paulussen et al. (Paulussen, Pharmacogenetics 10 (2000), 415-24) were detected in the screen of Caucasian samples and their allelic frequencies were estimated to be between 0.3% and 11.9% (Table 2A). 6 variants are located within the 6 kb sequence upstream of the transcriptional start site of CYP3A5. 14 variants are located in introns, or in the 5′-UTR or 3′-UTR, whereas 4 have been found in the protein-coding sequence. Among the latter ones, three variants result in amino acid substitutions and one in a premature termination of the CYP3A5 protein (Table 2A). The g.7303C>A variant (ch-v-009, Table 2A) results in a S100Y amino acid exchange in exon 4. The g.3705C>T variant (ch-v-005) leads to a H30Y amino acid exchange in exon 2. Cloning and sequencing revealed a physical linkage of this variant to the g.3709-3710insG variant (ch-v-006). The latter variant results in a shift of the open reading frame leading to a truncation of the protein sequence at position 34 (K34.). The T398N variant (ch-v-001, Table 2A), originally described by Jounaidi (Jounaidi, Biochem Biophys Res Commun 221 (1996), 466-70), was found in 3 out of 80 individuals tested.

Neither of the four protein altering variants found in Caucasians have been found in the African-Americans, Chinese, Japanese or Korean samples (Table 2A-E). However, among others we have found 4 new variants in these samples that result in an altered CYP3A5 amino acid sequence (ch-v-017, ch-v-043, ch-v-045, ch-v-068, Table 2B-E). The g.27131-27132insT (ch-v-017) variant in exon 11 (ch-v-017, Table 2B, 2D) has been found in 9 out of 45 African-American samples and in one out of 50 Japanese samples. The variant results in a shift of the open reading frame which leads to a truncation of the protein sequence at position 348 (D348.). Variants ch-v-043, ch-v-045 and ch-v-068 lead to amino acid exchanges.

### EXAMPLE 3

### Identification of Genetic Determinants of CYPA5 Protein Expression

In the following, the frequencies of Caucasian CYP3A5 gene variants have been analyzed as a function of CYP3A5 protein expression. For this purpose, allelic frequencies of variants shown in Table 2A were calculated separately for HE and LE livers (FIG. 3). The frequencies of 9 variants (ch-v-020, ch-v-021, ch-v-026, ch-v-034, ch-v-007, ch-v-008, ch-v-011, ch-v-014 and ch-v-015) were significantly increased in HE livers (all χ²>13.3, df=1, p<0.01, Bonferroni corrected). Except one, all tested HE livers (17/18, 94%) were heterozygous for three variants (ch-v-021, ch-v-026 and ch-v-015). 16 of those samples were heterozygous for ch-v-020 as well. One HE sample could not be genotyped for this variant. In contrast, LE livers were either wildtype (155/168, 92.3%), heterozygous for variants ch-v-021 and ch-v-26 (9/168, 5.4%) or heterozygous for the variant ch-v-015 (4/168, 2.4%) only. However, in LE livers all three variants never occurred simultaneously (Table 3). These results defined either of the three variants as a useful but imperfect marker of increased CYP3A5 expression. The variants ch-v-034, ch-v-008, ch-v-011 and ch-v-14 only occurred in a subset of the samples heterozygous for the above three variants (ch-v-021, ch-v-026 and ch-v-015).

The distribution of variants ch-v-021, ch-v-026 and ch-v-015 in the samples screened strongly suggest that they constitute a haplotype. In the following, the hypotheses whether these three variants recombine independently or not has been tested. Assuming their independent inheritance, the expected 3-loci-genotype frequencies for all combinations of variants and compared them with the observed frequencies have been calculated. The difference is highly significant (χ²=93.6; classes ‘all wildtype’, ‘single variant hetero- or homozygous’, ‘two or three variants hetero- or homozygous’; df=1; p<<0.001). There were more individuals with two or three of the variants than expected and less individuals with only one of the variants. This result suggests linkage among the three variants. The degree of linkage with the linkage disequilibrium parameter D for the three pairs of variants was estimated. Using maximum likelihood estimates for haplotype frequencies, D was calculated to be 0.041 for the variant pairs ch-v-021/ch-v-015 and ch-v-026/ch-v-015, which is 80% of its theoretical maximum, and 0.065 for variants ch-v-021 and ch-v-026 which corresponds to 100% of its theoretical maximum.

The probability that individuals showing the respective variant genotype are HE (positive predictive value) is estimated to be 65% for variants ch-v-021 and ch-v-026, respectively, and 81% for the ch-v-015 variant. For the combination of all three variants the positive predictive value is 100% in our sample set. However, assuming that these variants need to be located in cis for increased protein expression, it is clear that there is some probability for individuals showing all three variants to be LE. The results show that at least the allele ch-v-021/ch-v-026 and the allele ch-v-015 actually exist (see genotype 2 and 3, Table 3) and therefore the existence of a genotype with a combination of these two alleles has to be postulated. The maximum likelihood estimate for the frequency of these 3-fold heterozygotes having not all three variants in cis is 0.05% of all samples screened or 0.61% of samples hetero- or homozygote for all three variants. In other words, of 100 Caucasians screened statistically about 9 of them will be hetero- or homozygous for all three variants and about 0.05 of these will have not all three variants In cis. Therefore, it can be expected that the positive predictive power of the 3-variant genotype to be about 99.95%. Of course, the same values would be achieved for a combination of only two variants, either ch-v-021/ch-v-015 or ch-v-26/ch-v-15.

In a single HE liver none of the above 9 variants that were found in the other 17 HE samples could be detected. A closer examination of variants found in this sample revealed a variant within intron 4 (ch-v-018) and one within intron 5 (ch-v-019), respectively. These variants were unique to this sample, since they were not found in any other of the samples screened. Neither were they found in any of the other ethnic groups screened. It remains to be shown whether these variants are themselves causative for transcriptional activation or whether they are linked to another, so far undetected variant.

### EXAMPLE 4

### Determination of CYP3A5 Protein Expression

Protein expression of CYP3A4 and CYP3A5 in Caucasian liver samples was determined by Western blotting using CYP3A4- and CYP3A5-specific antibodies (Gentest). Liver microsomes were prepared as previously described (Zanger, Biochemistry 27 (1988), 5447-54). To obtain total protein homogenate, powdered liver tissue was homogenised in 0.1 M Tris-Cl pH 7.4, 1 mM EDTA, 1 mM Pefa Bloc SC, 1 μg/ml leupeptin, 1 μg/ml pepstatin with a Potter Elvehjem homogenisator (glass/Teflon) for 2 min at 1000 rpm. Homogenates were then sonified with a Bandelin Sonoplus HD 200 and stored at −80° C. For Western blotting, 12.5 μg microsomal protein homogenate or 40 μg total protein homogenate were separated in a 10% SDS-polyacrylamide gel. Electrophoretic transfer onto PVDF membranes was carried out in a TankBlot Cell (BioRad) for 1.5 hours at constant voltage (100 V) and at 10° C. Following the transfer, the membranes were incubated for 60 min in 5% milk, TBS, 0.1% Tween 20 to reduce the unspecific antibody binding. Incubations with either primary antibody (Gentest, dilution 1:500) were performed in 1% milk, TBS, 0.1% Tween 20 for 60 min, those with the secondary antibody (anti-rabbit IgG-POD Fab-fragments, Dianova, dilution 1:10000 in the same solution for 30 min. CYP3A4 or CYP3A5 protein bands were detected with Supersignal Dura (Pierce) and a digital CCD-camera (LAS-1000, Fuji). Signal quantification was performed with AIDA (Raytest). Protein expression levels were calculated based on calibration curves obtained with microsomes expressing recombinant CYP3A4 and CYP3A5 proteins (Gentest).

Homogenates or microsomal fractions were prepared from 186 human livers and investigated by Western blotting using a CYP3A5-specific antibody. CYP3A5 protein was detected in all samples analysed and its expression showed a clear bimodality (FIG. 1A). 168 livers (˜90%), further referred to as LE (low-expressing), showed expression close to or below the lower limit of quantification (LLOQ) of the assay (0.3 pmol/mg homogenate protein and 1.0 pmol/mg microsomal protein). Eighteen samples (˜10%), further referred to as HE (high-expressing), exhibited much higher CYP3A5 expression levels. The expression was in the range between 1.6 and 2.9 pmol/mg homogenate protein (2.3±0.5; n=6) and between 3.9 and 15.5 pmol/mg microsomal protein (9.7±4.1; n=12). Taking the LLOQ of the assay as the expression level of CYP3A5 in LE livers, HE livers express on average 8 to 10 times more CYP3A5 protein than LE livers.

In the following, the contribution of CYP3A5 to the combined CYP3A5 and CYP3A4 protein expression in HE livers was investigated. CYP3A4 expression in these livers was between 0.9 and 82.6 pmol/mg homogenate protein (n=6) and between 4.5 and 295 pmol/mg microsomal protein (n=11). The levels and the range of CYP3A4 variability in HE livers were similar to those in LE livers (not shown). FIG. 1B shows the share of CYP3A5 in the combined CYP3A5 and CYP3A4 protein pool in 17 HE livers. CYP3A5 contribution varies between 3% and 74%. In an average HE liver, the share of CYP3A5 in the combined pool of both proteins is 24%. Taking the LLOQ of the CYP3A5 assay as the actual expression level of the protein in LE livers, the corresponding value in these livers is approximately 1.6%.

### EXAMPLE 5

### Determination of CYP3A5 mRNA Expression

The expression of CYP3A5 mRNA in 8 Caucasian HE and 8 LE livers was investigated using a CYP3A5-specific TaqMan probe. As illustrated in FIG. 2A, the distribution of CYP3A5 mRNA levels exhibited a bimodality which was in complete agreement with that observed in the expression of CYP3A5 protein (Fisher's exact test, p=<<0.001). The number of 3A5 transcripts per ng of total RNA in HE livers (n=8) was on average 8.5 times higher than those in LE livers (n=8).

In the following, the allelic origin of CYP3A5 transcripts in HE and LE livers was investigated. To this end, by PCR a portion of the 3′-UTR (untranslated region) of the gene was amplified and sequenced using genomic or cDNA samples as templates which were heterozygous for a T>C variant located in this region (variant ch-v-015 in Table 2A;). As expected, both alleles are represented in the sequence using genomic DNAs as template (FIG. 2B). Both alleles were also equally represented in a sequence of PCR-amplified cDNA from a LE (homozygous wildtype for ch-v-021 and ch-v-26, heterozygous for ch-v-015) liver. In contrast, only the C allele was found in the same portion of CYP3A5 3′-UTR cDNA from a HE liver. This indicates an overrepresentation of transcripts derived from the chromosome harbouring the C allele in the total pool of CYP3A5 transcripts in HE livers.

### EXAMPLE 6

### In Vitro Mutagenesis and Expression of recombinant CYP3A5 Proteins

Five polymorphisms in Caucasians have been detected that lead to changes in the protein sequence. Two of them, ch-v-006 and ch-v-017, lead to a truncation of the protein and therefore are unlikely to code for a functional protein. As ch-v-005 has only been found physically linked to ch-v-006, the resulting protein variant is not likely to be functional as well. To determine the effect of the protein variants ch-v-009 and ch-v-001 these variants were analysed in a heterologous bacterial expression system.

A modified CYP3A5 cDNA in the prokaryotic expression vector pKK233-2 (Pharmacia) was used as starting material for in vitro mutagenesis (FIG. 5). Variants ch-v-009 and ch-v-001, respectively, were introduced into the plasmid by in vitro mutagenesis using the QuikChange mutagenesis kit (Stratagene). The successful introduction of the mutations and the absence of other, undesired mutations was confirmed by sequencing. The original plasmid as well as the two mutagenised plasmids were used to transform E. coli TOPP3 cells, a strain in which optimal expression of CYP3A proteins has previously been obtained. A total of 8 separate colonies of each mutant plasmid were chosen for expression studies. The bacteria were grown and induced as described in Eiselt et al. (Eiselt, Pharmacogenetics 11 (2001), 447-58.). Expression was analysed 48 h and 72 h after induction with IPTG/δ-ALA. Cells were harvested as described in Domanski et al. (Domanski, Arch Biochem Biophys 350 (1998), 223-32). The final P450 content was measured by reduced carbon monoxide (CO) difference spectra (Omura, J. Biol. Chem. 239 (1964), 2370-2378).

Whereas 30 to 50 nmol solubilised CYP3A5 could be recovered per litre culture of the non-mutagenised CYP3A5, expression in the two CYP3A5 variants S100Y and T398N was determined to be lower than 3 nmol P450 protein per litre culture. In many instances, the “P450” peak was shifted to 454-458 nm rather than the typical 448-450 nm peak expected. The low level of expression in mutagenised colonies made any attempts at protein purification futile. Previous experiments suggest that expression levels as low as those demonstrated by these CYP3A5 variants can not be significantly improved by utilising other bacterial strains or adjusting growth temperature. Therefore, the results strongly suggest that the CYP3A5 protein variants comprising the S100Y or the T398N substitutions are unstable in an E. coli expression system and that the variants comprising ch-v-009- or ch-v-001 may not code for functional proteins. The result of negative expression for variant ch-v-001 (T398N) is in agreement with the study in which this polymorphism was initially detected in two of five CYP3A5 deficient individuals (Jounaidi, Biochem Biophys Res Commun 221 (1996), 466-70).

### EXAMPLE 7

### Prediction of Expected Drug Metabolism by CYP3A5 Genotypes

The CYP3A5 protein degrades many drugs by oxidation so that they are not therapeutically active anymore. Therefore, drugs that are CYP3A5 substrates might not reach therapeutically active plasma concentrations for an adequate time span in patients with enhanced CYP3A5 activity. In these patients these drugs have to be dosed higher. On the other side, in patients with reduced CYP3A5 activity, these drugs have to be administered at lower dosage in order to avoid toxic drug levels. Table 4 gives an assignment for CYP3A5 genotypes and recommended dosages.

In cases in which CYP3A5 metabolism leads to the formation of pharmacologically active substance, enhanced enzyme activity has to be counteracted by reduced dosage whereas reduced CYP3A5 activity has to be met by increased dosage.

TABLE 1A

Primers used to screen for polymorphisms within
the CYP3A5 upstream regions and the CYP3A5 gene.

Primer

| | | | | | |
|---|---|---|---|---|---|
| Ref. | ID | Name | Sequence (5′-3′) | Position (nt) | (bp) |

| | | | | | |
|---|---|---|---|---|---|
| chzk | 001 | 694 | ACAGGCACAGAAACCCACAAG | 145448-145468¹ | 630 |
| | 002 | 711 | ATCGCCACTTGCCTTCTTC | 146077-146059¹ | |
| chzl | 003 | 794 | CCCTGCTTCGGCTTGTGCA | 159915-159933¹ | 575 |
| | 004 | 750 | CACAGCCTGCTTTATTTGTCATGA | 160489-160466¹ | |
| chzj | 005 | 751 | GATCCTTGGTAGGACAAGCCT | 160351-160371¹ | 844 |
| | 006 | 754 | CAAGCACTGATTTGGTCACTTCCT | 161194-161171¹ | |
| chzb | 007 | 819 | GGGATGGGACCGTAAGTGGAAC | 160951-160972¹ | 618 |
| | 008 | 820 | TAATCACATTGGAGTTCTGACAAATG | 161568-161543¹ | |
| chzi | 009 | 736 | AAAAACCTCTTACAAAAGTATCATCGGATA | 161419-161448¹ | 910 |
| | 010 | 737 | CCTACTAGGTCTCTGACTTGGAACCAT | 162328-162302¹ | |
| chzh | 011 | 784 | GCCGAGACGCACCATTACACT | 161876-161896¹ | 637 |
| | 012 | 785 | CACCCATCCCTTCCCACTCAT | 162512-162492¹ | |
| chzg | 013 | 740 | TGATGGTTCCAAGTCAGAGACCTAGTAG | 162300-162327¹ | 997 |
| | 014 | 741 | AATTGTAGACATCTTTCTCTTAAGTTAATTCCCAG | 163296-163262¹ | |
| chzf | 015 | 786 | TCTGCATGCCAACAGTCAACAATCT | 163182-163206¹ | 824 |
| | 016 | 789 | GGCACGCACCAGCATGTCC | 164005-163987¹ | |
| chze | 017 | 790 | CTGGCTGAGTGCCGTGGCT | 163845-163863¹ | 591 |
| | 018 | 791 | TGAGCGCTTCATGTATTCTGGCTAT | 164435-164411¹ | |
| chza | 019 | 824 | AAATATTTTCAAAGTCACACTCTGACAACAG | 164376-164406¹ | 617 |
| | 020 | 822 | TAACAGGATCTCATGCTTTTTTCATGGCT | 164992-164964¹ | |
| chzd | 021 | 747 | CACTCCAATATTCACAATAGCCACTATTCA | 164843-164872¹ | 926 |
| | 022 | 748 | ACTCCTACGTATCCTTCCAAGCCC | 165768-165745¹ | |
| chzc | 023 | 728 | GCTAAGGGAAACAGGCATAGAAACTTAC | 165586-165613¹ | 557 |
| | 024 | 729 | GGAGCTTCCCTGCCCTGC | 166142-166125¹ | |
| chzy | 025 | 323 | TCCTTCTCCAGCACATAAATC | 166076-166096¹ | 424 |
| | 026 | 325 | AAATTAGAAGGTGGATGGGAG | 166499-166479¹ | |
| chzx | 027 | 335 | GAGTAACTCACCAGCCCTCTG | 169838-169858¹ | 264 |
| | 028 | 336 | AAACCTCAGAACTCCCTCCCA | 170101-170081¹ | |
| chzw | 029 | 338 | GACATCTCTGAATAGCTTCCTTC | 171392-171414¹ | 402 |
| | 030 | 341 | GCACATAGTTTATAACGGCAA | 171793-171773¹ | |
| chzv | 031 | 346 | AGAACCTAAGGTTGCTGTGTGTC | 173303-173325¹ | 394 |
| | 032 | 348 | TGCAAGATGTTACCACTGGGC | 173696-173676¹ | |
| chzu | 033 | 354 | CGCCCCACATACACTCAGAA | 31376-31395² | 426 |
| | 034 | 357 | AGACCATTTTTAGGAAGCTCG | 31801-31781² | |
| chzt | 035 | 379 | CAAGGGGTACTCCACTGAGTTC | 31760-31781² | 403 |
| | 036 | 381 | CTCTTTGGAGTTGCAGCG | 32162-32145² | |
| chzs | 037 | 362 | AGGTGAGTCTAACTCAGCTTG | 33081-33101² | 578 |
| | 038 | 365 | GACAGCTAAAGTGTGTGAGGC | 33658-33638² | |
| chzr | 039 | 371 | AATGGGTTCCAGTTGAGAATC | 34411-34431² | 470 |
| | 040 | 373 | ATTGTTGTGCCTGATTTCAAG | 34880-34860² | |
| chzq | 041 | 387 | AGAAGCCATAGGCAGGTTG | 35627-35645² | 423 |
| | 042 | 389 | GACTGTCCTCCAAGCATTCT | 36049-36030² | |
| chzp | 043 | 394 | GATGCCATGATGAGGAGTGTG | 37724-37744² | 626 |
| | 044 | 397 | ACCAGGGCCAGCAATATTG | 38349-38331² | |
| chzm | 045 | 403 | AAATACTTCACGAATACTATGATCA | 45711-45735² | 595 |
| | 046 | 405 | CAGGGACATAATTGATTATCTTTG | 46305-46282² | |
| chzo | 047 | 411 | TACTGGTTGGGAGGTGGAG | 48290-48308² | 456 |
| | 048 | 412 | CATCATGTTCTTAATGCTACAGC | 48745-48723² | |
| chzn | 049 | 419 | GAAGAGTTCAAGATACATGGTGTTA | 50088-50112² | 416 |
| | 050 | 420 | TGCACAACACTCTACACAGACTC | 50503-50481² | |

Ref.: Reference sequence. The positions of primers refer to GenBank sequences with accession numbers (1) AF280107.1 and (2) AC005020.2.

TABLE 1B

Primers (Seq IDs 202 and 203) and probes
(Seq IDs 204 and 205) used determine the nucleotide
status at the polymorphic site ch-v-048
(g.6986G>A) by TaqMan assay.

Primer

| | | | | | |
|---|---|---|---|---|---|
| Ref. | ID | Name | Sequence (5′-3′) | Position (nt) | (bp) |

| | | | | | |
|---|---|---|---|---|---|
| chyu | 202 | TQPi_ch-v-048_F | GCTCTACTGTCATTTCTAACCATAAT | 173152-173184 | 99 |
| | | | CTCTTTA | | |
| | 204 | TQPo_ch-v- | VIC-TGTCTTTCAGTATCTCTT- | 173196-173213 | |
| | | 048_Al1_G_VIC | MGB-DQ | | |
| | 205 | TQPo_ch-v- | FAM-TGTCTTTCAATATCTCTTC- | 173196-173214 | |
| | | 048_Al2_A_FAM | MGB-DQ | | |
| | 203 | TQPi_ch-v-048_R | CCTTCATATGATGAAGGGTAATGTGG | 173250-173224 | |
| | | | T | | |

Ref.: Reference sequence. The positions of the primers refer to the GenBank sequence with accession number AF280107.1. Probes are labelled with a fluorenscent dye at the 5′ end and labelled with a dark quencher (DQ) and using a minor groove binder (MGB).

TABLE 2A

CYP3A5 polymorphisms detected in samples of Caucasian origin.

Caucasian

| | | |
|---|---|---|
| | | Variant |
| Variant position on | Sequence context | allele |

| | | | | | | | | | |
|---|---|---|---|---|---|---|---|---|---|
| Variant | Reference | Reference | | Seq | Reference seq. | Genetic | Predicted | | frequency |
| ID | sequence | sequence | gDNA | ID | Variant seq. | element | effect | N | (%) |

| | | | | | | | | | |
|---|---|---|---|---|---|---|---|---|---|
| ch-v-020 | chzk | 254T>G | g.−20619T>G | 051 | TGGGCTTGCAA | 5′ of PS2 | | 211 | 6.6 |
| | | | | 052 | .....G..... | | | | |
| ch-v-031 | chzk | 318G>A | g.−20555G>A | 053 | GCATGGGTAAA | 5′ of PS2 | | 189 | 0.3 |
| | | | | 054 | .....A..... | | | | |
| ch-v-032 | chzk | 544G>A | g.−20329G>A | 055 | GGGGTGTGTGC | 5′ of PS2 | | 186 | 0.3 |
| | | | | 056 | .....A..... | | | | |
| ch-v-033 | chzk | 550G>A | g.−20323G>A | 057 | TGTGCGATTCT | 5′ of PS2 | | 186 | 0.3 |
| | | | | 058 | .....A..... | | | | |
| ch-v-021 | chzk | 582A>G | g.−20291A>G | 059 | GCCCCACCTCC | 5′ of PS2 | | 215 | 6.7 |
| | | | | 060 | .....G..... | | | | |
| ch-v-026 | chzl | 229A>G | g.−6177A>G | 061 | CTCACACTGGG | 5′ of Exon 1 | | 208 | 7.0 |
| | | | | 062 | .....G..... | | | | |
| ch-v-027 | chzi | 566G>A | g.−4336G>A | 063 | GAGACGCACCA | 5′ of Exon 1 | | 19 | 2.7 |
| | | | | 064 | .....A..... | | | | |
| ch-v-028 | chzh | 601G>A | g.−3844G>A | 065 | TGTGTGTGGGA | 5′ of Exon 1 | | 20 | 10.0 |
| | | | | 066 | .....A..... | | | | |
| ch-v-029 | chzg | 464T>C | g.−3557T>C | 067 | ATCCATGTATA | 5′ of Exon 1 | | 20 | 2.5 |
| | | | | 068 | .....C..... | | | | |
| ch-v-034 | chza | 328T>C | g.−1617T>C | 069 | CATCTTACCCC | 5′ of Exon 1 | | 93 | 2.2 |
| | | | | 070 | .....C..... | | | | |
| ch-v-030 | chzd | 683T>A | g.−795T>A | 071 | TCTATTGCTAT | 5′ of Exon 1 | | 20 | 5.0 |
| | | | | 072 | .....A..... | | | | |
| ch-v-002 | chzy | 159G>A | g.−86G>A | 073 | GGCAGGGAAGC | Exon 1 (5′ UTR) | | 106 | 0.5 |
| | | | | 074 | .....A..... | | | | |
| ch-v-003 | chzy | 171C>T | g.−74C>T | 075 | CCAGGCAAACA | Exon 1 (5′ UTR) | | 106 | 4.3 |
| | | | | 076 | .....T..... | | | | |
| ch-v-004 | chzy | 418-420delGAG | g.174-176delGAG | 077 | TCAAGGAGAAG | Intron 1 | | 106 | 0.5 |
| | | | | 078 | .....---... | | | | |
| ch-v-005 | chzx | 187C>T | g.3705C>T | 079 | GTACACATGGA | Exon 2 | H30Y | 104 | 1.4 |
| | | | | 080 | .....T..... | | | | |
| ch-v-006 | chzx | 191-192insG | g.3709-3710insG | 081 | CATGG-ACTTT | Exon 2 | (K34.)¹ | 104 | 1.4 |
| | | | | 082 | .....G..... | | | | |
| ch-v-007 | chzw | 143C>T | g.5215C>T | 083 | GATAGCAGGCC | Intron 2 | | 105 | 6.7 |
| | | | | 084 | .....T..... | | | | |
| ch-v-048 | chyu | 206G>A | g.6986G>A | 146 | TTTCAGTATCT | Intron 3 | splice | 217 | 4.6 |
| | | | | 147 | .....A..... | | defect | | |
| ch-v-008 | chzv | 199C>A | g.7182C>A | 085 | AGAATCGGGCT | Intron 3 | | 107 | 1.9 |
| | | | | 086 | .....A..... | | | | |
| ch-v-009 | chzv | 320C>A | g.73030>A | 087 | TTATTCTGTCT | Exon 4 | S100Y | 107 | 0.5 |
| | | | | 088 | .....A..... | | | | |
| ch-v-018 | chzv | 441- | g.7424- | 089 | C--AG-----G | Intron 4 | | 107 | 0.5 |
| | | 444ins | 7427ins | 090 | CCTAAAAAATG | | | | |
| | | CTAAAAAAT | CTAAAAAAT | | | | | | |
| ch-v-016 | chzt | 145T>G | g.13077T>G | 091 | TCTTTTATCTT | Intron 5 | | 105 | 0.5 |
| | | | | 092 | .....G..... | | | | |
| ch-v-019 | chzt | 241T>C | g.13173T>C | 093 | GAGTCTGCACA | Intron 5 | | 105 | 0.5 |
| | | | | 094 | .....C..... | | | | |
| ch-v-010 | chzq | 132-133insGTC | g.16931- | 095 | AGTC---AAGA | Intron 8 | | 95 | 0.5 |
| | | | 16932insGTC | 096 | ....GTC.... | | | | |
| ch-v-011 | chzq | 364G>T | g.17163G>T | 097 | AGGAAGTATTC | Intron 9 | | 95 | 3.2 |
| | | | | 098 | .....T..... | | | | |
| ch-v-012 | chzp | 269G>A | g.19165G>A | 099 | AGAGAGCTTCA | Intron 9 | | 106 | 0.5 |
| | | | | 100 | .....A..... | | | | |
| ch-v-013 | chzm | 167A>G | g.27050A>G | 101 | CTTCAATAGTA | Intron 10 | | 80 | 11.9 |
| | | | | 102 | .....G..... | | | | |
| ch-v-001 | chzm | 4060>A | g.27289C>A | 103 | TCCAACTTATG | Exon 11 | T398N | 80 | 1.9 |
| | | | | 104 | .....A..... | | | | |
| ch-v-014 | chzm | 643C>T | g.27526C>T | 105 | CGAAACTACAT | Intron 11 | | 80 | 3.8 |
| | | | | 106 | .....T..... | | | | |
| ch-v-015 | chzn | 351T>C | g.31611T>C | 107 | AAGGATTTCTA | Exon 13 | | 197 | 5.6 |
| | | | | 108 | .....C..... | (3′ UTR) | | | |

All variants were detected in the heterozygous state except for 1 homozygous individual for variant ch-v-013. ¹variant results in a frame shift which ultimately leads to a premature termination.

TABLE 2B

CYP3A5 polymorphisms detected in samples of African-American origin

| | | |
|---|---|---|
| Variant position on | Sequence context | Variant |

| | | | | | | | | | |
|---|---|---|---|---|---|---|---|---|---|
| Variant | Reference | Reference | | Seq | reference seq. | Genetic | Predicted | | allele |
| ID | sequence | sequence | gDNA | ID | variant seq. | element | effect | N | frequency (%) |

| | | | | | | | | | |
|---|---|---|---|---|---|---|---|---|---|
| ch-v-037 | chzk | 230T>C | g.−20643T>C | 148 | TTTAATAGAAG | 5′ of PS2 | | 42 | 3.6 |
| | | | | 149 | .....C..... | | | | |
| ch-v-020 | chzk | 254T>G | g.−20619T>G | 051 | TGGGCTTGCAA | 5′ of PS2 | | 41 | 69.5 |
| | | | | 052 | .....G..... | | | | |
| ch-v-038 | chzk | 506C>T | g.−20367C>T | 150 | ATTCCCCATAG | 5′ of PS2 | | 44 | 1.1 |
| | | | | 151 | .....T..... | | | | |
| ch-v-039 | chzk | 514T>C | g.−20359T>C | 152 | TAGAATATGAA | 5′ of PS2 | | 44 | 1.1 |
| | | | | 153 | .....C..... | | | | |
| ch-v-021 | chzk | 582A>G | g.−20291A>G | 059 | GCCCCACCTCC | 5′ of PS2 | | 45 | 66.7 |
| | | | | 060 | .....G..... | | | | |
| ch-v-026 | chzl | 229A>G | g.−6177A>G | 061 | CTCACACTGGG | 5′ of Exon 1 | | 43 | 65.1 |
| | | | | 062 | .....G..... | | | | |
| ch-v-051 | chzh | 455T>G | g.−3990T>G | 154 | GTAACTTATCC | 5′ of Exon 1 | | 44 | 2.3 |
| | | | | 155 | .....G..... | | | | |
| ch-v-052 | chzh | 577G>A | g.−3868G>A | 156 | TTCACGTGGAG | 5′ of Exon 1 | | 44 | 3.4 |
| | | | | 157 | .....A..... | | | | |
| ch-v-028 | chzh | 601G>A | g.−3844G>A | 065 | TGTGTCTGGGA | 5′ of Exon 1 | | 44 | 17.1 |
| | | | | 066 | .....A..... | | | | |
| ch-v-034 | chyz | 328T>C | g.−1617T>C | 069 | CATCTTACCCC | 5′ of Exon 1 | | 45 | 42.2 |
| | | | | 070 | .....C..... | | | | |
| ch-v-002 | chzy | 159G>A | g.−86G>A | 073 | GGCACGCAAGC | Exon 1 (5′ UTR) | | 45 | 1.1 |
| | | | | 074 | .....A..... | | | | |
| ch-v-003 | chzy | 171C>T | g.−74C>T | 075 | CCAGGCAAACA | Exon 1 (5′ UTR) | | 45 | 1.1 |
| | | | | 076 | .....T..... | | | | |
| ch-v-007 | chzw | 1430>T | g.5215C>T | 083 | GATAGCAGGCC | Intron 2 | | 44 | 3.4 |
| | | | | 084 | .....T..... | | | | |
| ch-v-053 | chzw | 163G>A | g.5235G>A | 158 | TGGACGCAACT | Intron 2 | | 45 | 2.2 |
| | | | | 159 | .....A..... | | | | |
| ch-v-054 | chzw | 444T>A | g.5516T>A | 160 | GAGGATAATTA | Intron 3 | | 43 | 3.5 |
| | | | | 161 | .....A..... | | | | |
| ch-v-048 | chyu | 206G>A | g.6986G>A | 146 | TTTCAGTATCT | Intron 3 | | 45 | 73.3 |
| | | | | 147 | .....A..... | | | | |
| ch-v-025 | chzv | 224C>T | g.7207C>T | 109 | AGCTCCGTTGT | Intron 3 | | 43 | 7.0 |
| | | | | 110 | .....T..... | | | | |
| ch-v-043 | chzt | 294T>C | g.13226T>C | 162 | CATCATTGCCC | Exon 6 | I149T | 45 | 1.1 |
| | | | | 163 | .....C..... | | | | |
| ch-v-055 | chzt | 444G>A | g.13376G>A | 164 | CAGTCGCACTG | Intron 6 | | 45 | 1.1 |
| | | | | 165 | .....A..... | | | | |
| ch-v-050 | chzs | 437G>A | g.14690G>A | 166 | ACTAAGAAGTT | Exon 7 | splice | 45 | 13.3 |
| | | | | 167 | .....A..... | | defect | | |
| ch-v-056 | chzs | 467A>G | g.14720A>G | 168 | GATCCATTATT | Exon 7 | P218P | 45 | 6.7 |
| | | | | 169 | .....G..... | | | | |
| ch-v-057 | chzs | 583C>T | g.14836C>T | 170 | CAATTCCATTG | Intron 7 | | 45 | 1.1 |
| | | | | 171 | .....T..... | | | | |
| ch-v-058 | chzs | 650A>G | g.14903A>G | 172 | TGTCAATCTAG | Intron 7 | | 45 | 6.7 |
| | | | | 173 | .....C..... | | | | |
| ch-v-059 | chzr | 205T>C | g.15788T>C | 174 | TTGTTTGTTTT | Intron 7 | | 44 | 3.4 |
| | | | | 175 | .....C..... | | | | |
| ch-v-060 | chzr | 496A>C | g.16079A>C | 176 | AAATAAAGAAC | Intron 8 | | 44 | 1.1 |
| | | | | 177 | .....C..... | | | | |
| ch-v-011 | chzq | 364G>T | g.17163G>T | 097 | AGGAAGTATTC | Intron 9 | | 43 | 7.0 |
| | | | | 098 | .....T..... | | | | |
| ch-v-062 | chzp | 173G>A | g.19069G>A | 178 | TTTGCGTCATC | Intron 9 | | 45 | 1.1 |
| | | | | 179 | .....A..... | | | | |
| ch-v-063 | chzp | 312C>T | g.19208C>T | 180 | TTGACCTGATT | Intron 9 | | 45 | 2.2 |
| | | | | 181 | .....T..... | | | | |
| ch-v-013 | chzm | 167A>G | g.27050A>G | 101 | CTTCAATAGTA | Intron 10 | | 36 | 1.4 |
| | | | | 102 | .....G..... | | | | |
| ch-v-017 | chzm | 248-249 | g.27131-27132 | 111 | CACCT-ACCTA | Exon 11 | (D348.)¹ | 45 | 10.0 |
| | | insT | insT | 112 | .....T..... | | | | |
| ch-v-014 | chzm | 643C>T | g.27526C>T | 105 | CGAAACTACAT | Intron 11 | | 42 | 11.9 |
| | | | | 106 | .....T..... | | | | |
| ch-v-044 | chzn | 239T>C | g.31499T>C | 182 | TATTGTAGATC | Intron 12 | | 45 | 5.6 |
| | | | | 183 | .....C..... | | | | |
| ch-v-015 | chzn | 351T>C | g.31611T>C | 107 | AAGGATTTCTA | Exon 13 | | 44 | 68.2 |
| | | | | 108 | .....C..... | (3′ UTR) | | | |

¹variant results in a frame shift which ultimately leads to premature termination.

TABLE 2C

CYP3A5 polymorphisms detected in samples of Chinese origin.

| | | |
|---|---|---|
| Variant position on | Sequence context | Variant |

| | | | | | | | | | |
|---|---|---|---|---|---|---|---|---|---|
| Variant | Reference | Reference | | Seq | reference seq. | Genetic | Predicted | | allele |
| ID | sequence | sequence | gDNA | ID | variant seq. | element | effect | N | frequency (%) |

| | | | | | | | | | |
|---|---|---|---|---|---|---|---|---|---|
| ch-v-020 | chzk | 254T>G | g.−20619T>G | 051 | TGGGCTTGCAA | 5′ of PS2 | | 42 | 23.8 |
| | | | | 052 | .....C..... | | | | |
| ch-v-021 | chzk | 582A>G | g.−20291A>G | 059 | GCCCCACCTCC | 5′ of PS2 | | 45 | 26.7 |
| | | | | 060 | .....G..... | | | | |
| ch-v-026 | chzl | 229A>G | g.−6177A>G | 061 | CTCACACTGGG | 5′ of Exon 1 | | 47 | 26.6 |
| | | | | 062 | .....G..... | | | | |
| ch-v-034 | chyz | 328T>C | g.−1617T>C | 069 | CATCTTACCCC | 5′ of Exon 1 | | 47 | 21.3 |
| | | | | 070 | .....C..... | | | | |
| ch-v-066 | chzy | 380T>C | g.136T>C | 184 | CCTTTTCCCTT | Intron 1 | | 45 | 1.1 |
| | | | | 185 | .....C..... | | | | |
| ch-v-067 | chzy | 474G>A | g.230G>A | 186 | CTTATGCAGAT | Intron 1 | | 44 | 1.14 |
| | | | | 187 | .....A..... | | | | |
| ch-v-048 | chyu | 206G>A | g.6986G>A | 146 | TTTCAGTATCT | Intron 3 | | 47 | 26.6 |
| | | | | 147 | .....A..... | | | | |
| ch-v-018 | chzv | 441- | g.7424- | 089 | C--AG-----C | Intron 4 | | 47 | 2.1 |
| | | 444ins | 7427ins | 090 | CCTAAAAAATG | | | | |
| | | CTAAAAAAT | CTAAAAAAT | | | | | | |
| ch-v-068 | chzu | 359G>A | g.12907G>A | 188 | AATACGGTCAT | Exon 5 | R130Q | 45 | 3.3 |
| | | | | 189 | .....A..... | | | | |
| ch-v-047 | chzu | 404T>C | g.12952T>C | 190 | GGAGGTATGAA | Intron 5 | splice | 44 | 1.1 |
| | | | | 191 | .....C..... | | defect | | |
| ch-v-069 | chzu | 480G>A | g.13028G>A | 192 | AGTCCGTTTCC | Intron 5 | | 44 | 1.1 |
| | | | | 193 | .....A..... | | | | |
| ch-v-011 | chzq | 364G>T | g.17163G>T | 097 | AGGAAGTATTC | Intron 9 | | 40 | 21.3 |
| | | | | 098 | .....T..... | | | | |
| ch-v-014 | chzm | 643C>T | g.27526C>T | 105 | CGAAACTACAT | Intron 11 | | 47 | 5.3 |
| | | | | 106 | .....T..... | | | | |
| ch-v-015 | chzn | 351T>C | g.31611T>C | 107 | AAGGATTTCTA | Exon 13 | | 47 | 26.6 |
| | | | | 108 | .....C..... | (3′ UTR) | | | |

TABLE 2D

CYP3A5 polymorphisms detected in samples of Japanese origin.

| | | |
|---|---|---|
| Variant position on | Sequence context | Variant |

| | | | | | | | | | |
|---|---|---|---|---|---|---|---|---|---|
| Variant | Reference | Reference | | Seq | reference seq. | Genetic | Predicted | | allele |
| ID | sequence | sequence | gDNA | ID | variant seq. | element | effect | N | frequency (%) |

| | | | | | | | | | |
|---|---|---|---|---|---|---|---|---|---|
| ch-v-020 | chzk | 254T>G | g.−20619T>G | 051 | TGGGCTTGCAA | 5′ of PS2 | | 42 | 29.8 |
| | | | | 052 | .....C..... | | | | |
| ch-v-021 | chzk | 582A>G | g.−20291A>G | 059 | GCCCCACCTCC | 5′ of PS2 | | 49 | 28.6 |
| | | | | 060 | .....G..... | | | | |
| ch-v-026 | chzl | 229A>G | g.−6177A>G | 061 | CTCACACTGGG | 5′ of Exon 1 | | 46 | 28.3 |
| | | | | 062 | .....C..... | | | | |
| ch-v-028 | chzh | 601G>A | g.−3844G>A | 065 | TGTGTGTGGGA | 5′ of Exon 1 | | 50 | 2.0 |
| | | | | 066 | .....A..... | | | | |
| ch-v-034 | chyz | 328T>C | g.−1617T>C | 069 | CATCTTACCCC | 5′ of Exon 1 | | 50 | 26.0 |
| | | | | 070 | .....C..... | | | | |
| ch-v-007 | chzw | 143C>T | g.5215C>T | 083 | GATACCACGCC | Intron 2 | | 48 | 3.1 |
| | | | | 084 | .....T..... | | | | |
| ch-v-048 | chyu | 206G>A | g.6986G>A | 146 | TTTCAGTATCT | Intron 3 | | 50 | 29.0 |
| | | | | 147 | .....A..... | | | | |
| ch-v-047 | chzu | 404T>C | g.12952T>C | 190 | GGAGGTATGAA | Intron 5 | splice | 50 | 1.0 |
| | | | | 191 | .....C..... | | defect | | |
| ch-v-061 | chzq | 194C>G | g.16993C>G | 194 | TCTGCCAAAGA | Intron 8 | | 49 | 1.0 |
| | | | | 195 | .....G..... | | | | |
| ch-v-011 | chzq | 364G>T | g.17163G>T | 097 | AGGAACTATTC | Intron 9 | | 49 | 26.5 |
| | | | | 098 | .....T..... | | | | |
| ch-v-017 | chzm | 248-249insT | g.27131-27132insT | 111 | CACCT-ACCTA | Exon 11 | (D348.)¹ | 48 | 1.0 |
| | | | | 112 | .....T..... | | | | |
| ch-v-014 | chzm | 643C>T | g.27526C>T | 105 | CGAAACTACAT | Intron 11 | | 49 | 3.1 |
| | | | | 106 | .....T..... | | | | |
| ch-v-045 | chzn | 291T>C | g.31551T>C | 196 | ACCCATTGTTC | Exon 13 | I488T | 50 | 3.0 |
| | | | | 197 | .....C..... | | | | |
| ch-v-015 | chzn | 351T>C | g.31611T>C | 107 | AAGGATTTCTA | Exon 13 | | 50 | 31.0 |
| | | | | 108 | .....C..... | (3′ UTR) | | | |

TABLE 2E

CYP3A5 polymorphisms detected in samples of Korean origin.

| | | |
|---|---|---|
| Variant position on | Sequence context | Variant |

| | | | | | | | | | |
|---|---|---|---|---|---|---|---|---|---|
| Variant | Reference | Reference | | Seq | reference seq. | Genetic | Predicted | | allele |
| ID | sequence | sequence | gDNA | ID | variant seq. | element | effect | N | frequency (%) |

| ch-v-020 | chzk | 254T>G | g.−20619T>G | 051 | TGGGCTTGCAA | 5′ of PS2 | 47 | 29.8 |
|---|---|---|---|---|---|---|---|---|
| | | | | 052 | .....G..... | | | |
| ch-v-065 | chzk | 563T>C | g.−20310T>C | 198 | GCTACTGGCTG | 5′ of PS2 | 47 | 1.1 |
| | | | | 199 | .....C..... | | | |
| ch-v-021 | chzk | 582A>G | g.−20291A>G | 059 | GCCCCACCTCC | 5′ of PS2 | 47 | 29.8 |
| | | | | 060 | .....G..... | | | |
| ch-v-040 | chzl | 206C>T | g.−62000>T | 200 | GAAATCACCCG | 5′ of Exon 1 | 43 | 1.2 |
| | | | | 201 | .....T..... | | | |
| ch-v-026 | chzl | 229A>G | g.−6177A>G | 061 | CTCACACTGCG | 5′ of Exon 1 | 43 | 31.4 |
| | | | | 062 | .....G..... | | | |
| ch-v-034 | chyz | 328T>C | g.−1617T>C | 069 | CATCTTACCCC | 5′ of Exon 1 | 47 | 27.7 |
| | | | | 070 | .....C..... | | | |
| ch-v-007 | chzw | 143C>T | g.5215C>T | 083 | CATAGCAGCCC | Intron 2 | 47 | 2.1 |
| | | | | 084 | .....T..... | | | |
| ch-v-048 | chyu | 206G>A | g.6986G>A | 146 | TTTCAGTATCT | Intron 3 | 47 | 29.8 |
| | | | | 147 | .....A..... | | | |
| ch-v-061 | chzq | 194C>G | g.16993C>G | 194 | TCTGCCAAAGA | Intron 8 | 47 | 2.1 |
| | | | | 195 | .....G..... | | | |
| ch-v-011 | chzq | 364G>T | g.17163G>T | 097 | AGGAAGTATTC | Intron 9 | 47 | 27.7 |
| | | | | 098 | .....T..... | | | |
| ch-v-014 | chzm | 643C>T | g.27526C>T | 105 | CGAAACTACAT | Intron 11 | 47 | 2.1 |
| | | | | 106 | .....T..... | | | |
| ch-v-015 | chzn | 351T>C | g.31611T>G | 107 | AAGGATTTCTA | Exon 13 | 43 | 36.1 |
| | | | | 108 | .....C..... | (3′ UTR) | | |
| Table 2A-E: Variants are listed according to their localisation along the gene, separately for each ethnic group. Polymorphism nomenclature is based on Antoarakis et al. (Antonarakis, Hum Mutat 11 (1998), 1-3) using the joined sequences AF280107.1 and AC005020.2 as genomic reference sequences wherein the A of the ATG at position 166220 in AF280107.1 is +1. Sequence context: local alignment at the polymorphic site with the reference allele sequence given at the top and the | | | | | | | | |
| # variant sequence given below. Dots indicate nucleotide identity at the respective position. N: number of samples analysed. | | | | | | | | |

TABLE 3
CYP3A5 genotypes and phenotypes.

| | ch-v-021 | ch-v-026 | ch-v-015 | Phenotype | Livers |
|---|---|---|---|---|---|
| Genotype 1 | A/A | A/A | T/T | LE | 155 |
| Genotype 2 | A/G | A/G | T/T | LE | 9 |
| Genotype 3 | A/A | A/A | T/C | LE | 4 |
| Genotype 4 | A/G | A/G | T/C | HE | 17 |
| Genotype 5 | A/A | A/A | T/T | HE | 1 |

All three variants were observed only in the heterozygous state.
HE = high expressing livers,
LE = low expressing livers. Numbers indicate LE and HE livers with each particular genotype. The increased CYP3A5 expression co-segregates with a distinct genotype.

TABLE 4
Expected drug metabolism by CYP3A5

| | | | Dos Adjustment | |
|---|---|---|---|---|
| Genotype | | Enzyme | drug | drug |
| No. | | Activity | degradation | activation |
| | Allelic combination | | | |
| I | high expressor allele/ | 190% | 1.90 | 0.53 |
| | high expressor allele | | | |
| II, III | low expressor allele/ | 100% | 1.00 | 1.00 |
| | high expressor allele | | | |
| IV-VII | null allele/ | 95% | 0.95 | 1.05 |
| | high expressor allele | | | |
| VIII, | low expressor allele/ | 10% | 0.10 | 10 |
| IX, X | low expressor allele | | | |
| XI-XVIII | null allele/ | 5% | 0.05 | 20 |
| | low expressor allele | | | |
| XIX-XXV | null allele/ | 0% | <0.05 | >20 |
| | null allele | | | |
| | Genotype (SeqID) at Locus | | | |
| | 1-2-3-4-5-6-7-8 | | | |
| I | 060-062-079-081-087-111-103-108/ | 190% | 1.90 | 0.53 |
| | 060-062-079-081-087-111-103-108 | | | |
| II | 0xx-06x-079-081-087-111-103-107/ | 100% | 1.00 | 1.00 |
| | 060-062-079-081-087-111-103-108 | | | |
| III | 059-061-079-081-087-111-103-10x/ | 100% | 1.00 | 1.00 |
| | 060-062-079-081-087-111-103-108 | | | |
| IV | 0xx-06x-080-082-08x-11x-10x-10x/ | 95% | 0.95 | 1.05 |
| | 060-062-079-081-087-111-103-108 | | | |
| V | 0xx-06x-0xx-08x-088-11x-10x-10x/ | 95% | 0.95 | 1.05 |
| | 060-062-079-081-087-111-103-108 | | | |
| VI | 0xx-06x-0xx-08x-08x-112-10x-10x/ | 95% | 0.95 | 1.05 |
| | 060-062-079-081-087-111-103-108 | | | |
| VII | 0xx-06x-0xx-08x-08x-11x-104-10x/ | 95% | 0.95 | 1.05 |
| | 060-062-079-081-087-111-103-108 | | | |
| VIII | 060-062-079-081-087-111-103-107/ | 10% | 0.10 | 10 |
| | 059-061-079-081-087-111-103-108 | | | |
| IX | 0xx-06x-079-081-087-111-103-107/ | 10% | 0.10 | 10 |
| | 0xx-06x-079-081-087-111-103-107 | | | |
| X | 059-061-079-081-087-111-103-10x/ | 10% | 0.10 | 10 |
| | 059-061-079-081-087-111-103-10x | | | |
| XI | 059-061-079-081-087-111-103-10x/ | 5% | 0.05 | 20 |
| | 0xx-06x-080-082-08x-11x-10x-10x | | | |
| XII | 0xx-06x-079-081-087-111-103-107/ | 5% | 0.05 | 20 |
| | 0xx-06x-080-082-08x-11x-10x-10x | | | |
| XIII | 059-061-079-081-087-111-103-10x/ | 5% | 0.05 | 20 |
| | 0xx-06x-0xx-08x-088-11x-10x-10x | | | |
| XIV | 0xx-06x-079-081-087-111-103-107/ | 5% | 0.05 | 20 |
| | 0xx-06x-0xx-08x-088-11x-10x-10x | | | |
| XV | 059-061-079-081-087-111-103-10x/ | 5% | 0.05 | 20 |
| | 0xx-06x-0xx-08x-08x-112-10x-10x | | | |
| XVI | 0xx-06x-079-081-087-111-103-107/ | 5% | 0.05 | 20 |
| | 0xx-06x-0xx-08x-08x-112-10x-10x | | | |
| XVII | 059-061-079-081-087-111-103-10x/ | 5% | 0.05 | 20 |
| | 0xx-06x-0xx-08x-08x-11x-104-10x | | | |
| XVIII | 0xx-06x-079-081-087-111-103-107/ | 5% | 0.05 | 20 |
| | 0xx-06x-0xx-08x-08x-11x-104-10x | | | |
| XIX | 0xx-06x-080-082-08x-11x-10x-10x/ | 0% | <0.05 | >20 |
| | 0xx-06x-0xx-08x-088-11x-10x-10x | | | |
| XX | 0xx-06x-080-082-08x-11x-10x-10x/ | 0% | <0.05 | >20 |
| | 0xx-06x-0xx-08x-08x-112-10x-10x | | | |
| XXI | 0xx-06x-080-082-08x-11x-10x-10x/ | 0% | <0.05 | >20 |
| | 0xx-06x-0xx-08x-08x-11x-104-10x | | | |
| XXII | 0xx-06x-080-082-08x-11x-10x-10x/ | 0% | <0.05 | >20 |
| | 0xx-06x-080-082-088-11x-10x-10x | | | |
| XXIII | 0xx-06x-0xx-08x-088-11x-10x-10x/ | 0% | <0.05 | >20 |
| | 0xx-06x-0xx-08x-08x-11x-10x-10x | | | |
| XXIV | 0xx-06x-0xx-08x-08x-112-10x-10x/ | 0% | <0.05 | >20 |
| | 0xx-06x-0xx-08x-08x-112-10x-10x | | | |
| XXV | 0xx-06x-0xx-08x-08x-11x-104-10x/ | 0% | <0.05 | >20 |
| | 0xx-06x-0xx-08x-08x-11x-104-10x | | | |
| No.: running genotype number. | | | | |
| Genotype: Possible CYP3A5 genotypes that result from combinations of alleles. At the top of the table concise allele names have been used to indicate the principle. The lower table lists the alleles in greater detail, giving all combinations of variants at 8 loci in the two homologous chromosomes (loci 1-8 refer to positions corresponding to positions −20291, −6177, 3705, 3709/3710, 7303, 27131/27132, 27289 and 31611, respectively, of the CYP3A5 gene (Accession No: AF280107.1, | | | | |
| # wherein position 166220 has been numbered +1 and position 174832 has been numbered +8613), respectively. Each variant is defined by a 3-digit Seq ID as listed in Table 2A-E. A wildcard (x) in Seq IDs indicates that the phenotype is independent from the variant at this locus in this chromosome. The possible variants for each locus that can be substituted for x can be extracted from Table 2A-E. For example, 08x at locus 4 stands for Seq IDs 081 or 082, whereas 08x at locus 5 indicates | | | | |
| # either Seq ID 087 or 088. | | | | |

Enzyme Activity: enzyme activity as calculated from protein concentration whereby the average protein concentration of genotype 059-061-079-081-111-107/060-062-079-081-111-108 was defined as 100%.
Dose Adjustment: dose adjustment factors for drugs that are degraded/activated by CYP3A5 relative to the dosis required for genotype 059-061-079-081-111-107/060-062-079-081-111-108. Factors may need to be weighted according to the activity share of the CYP3A5 enzyme for drugs which are not exclusively metabolised by CYP3A5.

1. A polynucleotide comprising a polynucleotide selected from the group consisting of:
(a) a polynucleotide having the nucleic acid sequence of SEQ ID NO: 54, 56, 58, 62, 64, 66, 68, 70, 72, 74, 76, 78, 80, 82, 84, 86, 88, 90, 92, 94, 96, 98, 100, 102, 106, 108, 110, 112, 113, 114, 115, 116, 117, 118 119, 120, 121, 122, 123, 124, 125, 126, 128, 129, 130, 131, 133, 134, 135, 136, 137, 138, 139, 140, 142, 143, 149, 151, 153, 155, 157, 159, 161, 163, 165, 169, 171, 173, 175, 177, 179, 181, 183, 185, 187, 189, 193, 195, 197, 199, 201, 207, 208, 209, 210, 211, 212, 213, 214, 216, 218, 219, 220, 221, 222, 223, 224, 225, 226, 227, 228, 231, 232, 233, 235, or 236;
(b) a polynucleotide encoding a polypeptide having the amino acid sequence of SEQ ID NO: 127, 132, 141, 215, 229, or 234;
(c) a polynucleotide capable of hybridizing to a CYP3A5 gene, wherein said polynucleotide is having a nucleotide exchange, a nucleotide deletion of at least one nucleotide, or at least one additional nucleotide at a position corresponding to position -20643, -20555, -20359, -20367, -20329, -20323, -20310, -6200, -6177, -4336, -3990, -3868, -3844, -3557, -1617, -795, -86, -74, 136, 174 to 176, 230, 3705, 3709/3710, 5215, 5235, 5516, 7182, 7207, 7303, 7424/7427, 12907,13028, 13077, 13173, 13226, 13376, 14720, 14836, 14903, 15788, 16079, 16931/16932, 16993, 17163, 19069, 19165, 19208, 27050, 27131/27132, 27526, 31499, 31551 or 31611;
(d) a polynucleotide capable of hybridizing to a CYP3A5 gene, wherein said polynucleotide is having an A at a position corresponding to position -20555, -20329, -20323, -4336, -3868, -3844, -795, -86, 230, 5235, 5516, 7182, 7303, 12907, 13028, 13376, 19069 or 19165 of the CYP3A5 gene (Accession No: AF280107.1-, wherein position 166220 has been numbered +1 and position 174832 has been numbered +8613, and Accession No: AC005020.2, wherein position 27341 has been numbered +8614), a T at a position corresponding to position -20367, -6200, -74, 3705, 5215, 7207, 14836, 17163, 19208 or 27526 of the CYP3A5 gene (Accession No: AF280107.1, wherein position 166220 has been numbered +1 and position 174832 has been numbered +8613, and Accession No: AC005020.2, wherein position 27341, has been numbered +8614), a G at a position corresponding to position -6177, -3990, 13077, 14720, 14903, 16993 or 27050 of the CYP3A5 gene (Accession No: AF280107.1, wherein position 166220 has been numbered +1 and position 174832 has been numbered +8613, and Accession No: AC005020.2, wherein position 27341 has been numbered +8614), a C at a position corresponding to position -20643, -20310, -3557, -1617, 136, 13173, 13226, 15788, 16079, 31499, 31551 or 31611 of the CYP3A5 gene (Accession No: AF280107.1, wherein position 166220 has been numbered +1 and position 174832 has been numbered +8613, and Accession No: AC005020.2, wherein position 27341 has been numbered +8614), nucleotide deletions at positions corresponding to positions 174 to 176 of the CYP3A5 gene (Accession No: AF280107.1, wherein position 166220 has been numbered +1 and position 174832 has been numbered +8613), an additional nucleotide at a position corresponding to position 3709/3710 or 27131/27132 of the CYP3A5 gene (Accession No: AF280107.1, wherein position 166220 has been numbered +1 and position 174832 has been numbered +8613, and Accession No: AC005020.2, wherein position 27341 has been numbered +8614), three additional nucleotides at a position corresponding to position 16931/16932 of the CYP3A5 gene (Accession No: AF280107.1, wherein position 166220 has been numbered +1 and position 174832 has been numbered +8613, and Accession No: AC005020.2, wherein position 27341 has been numbered +8614), or a deletion of two nucleotides and nine additional nucleotides inserted at a position corresponding to position 7424 to 7427 of the CYP3A5 gene (Accession No: AF280107.1, wherein position 166220 has been numbered +1 and position 174832 has been numbered +8613);
(e) a polynucleotide encoding a CYP3A5 polypeptide or fragment thereof, wherein said polypeptide comprises an amino acid substitution at a position corresponding to position 30, 100, 130, 149 or 488 of the CYP3A5 polypeptide (Accession No: NP_{—}000768.1), or at least one amino acid exchange or a stop codon at a position corresponding to position 30 to 34 or 346 to 348 of the CYP3A5 polypeptide (Accession No: NP_{—}000768.1); and
(f) a polynucleotide encoding a CYP3A5 polypeptide or fragment thereof, wherein said polypeptide comprises amino acid substitutions of HGLFK to YGTF. (with the period meaning termination) at a position corresponding to position 30 to 34 of the CYP3A5 polypeptide (Accession No: NP_{—}000768.1), an amino acid substitution of S to Y at a position corresponding to position 100 of the CYP3A5 polypeptide (Accession No: NP_{—}000768.1), an amino acid substitution of R to Q at a position corresponding to position 130 of the CYP3A5 polypeptide (Accession No: NP_{—}000768.1), an amino acid substitution of I to T at a position corresponding to position 149 of the CYP3A5 polypeptide (Accession No: NP_{—}000768.1), an amino acid substitutions of TYD to YL. (with the period meaning termination) at position corresponding to position 346 to 348 of the CYP3A5 polypeptide (Accession No: NP_{—}000768.1), or an amino acid substitution of I to T at a position corresponding to position 488 of the CYP3A5 polypeptide (Accession No: NP_{—}000768.1).

2. A polynucleotide of claim 1, wherein said polynucleotide is associated with cancer or diseases including cardiovascular diseases, diabetes and AIDS.

3. A polynucleotide of any one of claims 1 to 2 which is DNA or RNA.

4. A gene comprising the polynucleotide of any one of claims 1 to 2.

5. The gene of claim 4 wherein a nucleotide deletion, addition and/or substitution results in altered expression of the variant gene compared to the corresponding wild type gene.

6. A vector comprising a polynucleotide of any one of claims 1 to 3 or the gene of claim 4 or 5.

7. The vector of claim 6, wherein the polynucleotide is operatively linked to expression control sequences allowing expression in prokaryotic or eukaryotic cells or isolated fractions thereof.

8. A host cell genetically engineered with the polynucleotide of any one of claims 1 to 3, the gene of claim 4 or 5 or the vector of claim 6 or 7.

9. A method for producing a molecular variant CYP3A5 polypeptide or fragment thereof comprising
(a) culturing the host cell of claim 8; and
(b) recovering said protein or fragment from the culture.

10. A method for producing cells capable of expressing a molecular variant CYP3A5 polypeptide comprising genetically engineering cells with the polynucleotide of any one of claims 1 to 3, the gene of claim 4 or 5 or the vector of claim 6 or 7.

11. A polypeptide or fragment thereof encoded by the polynucleotide of any one of claims 1 to 3, the gene of claim 4 or 5 or obtainable by the method of claim 9 or from cells produced by the method of claim 10.

12. An antibody which binds specifically to the polypeptide of claim 11.

13. The antibody of claim 12 which specifically recognizes an epitope containing one or more amino acid substitution(s) resulting from a nucleotide exchange as defined in claim 1 or 5.

14. The antibody of claim 12 or 13 which is monoclonal or polyclonal.

15. A transgenic non-human animal comprising at least one polynucleotide of any one of claims 1 to 4, the gene of claim 5 or 6 or the vector of claim 7 or 8.

16. The transgenic non-human animal of claim 15 which is a mouse, a rat or a zebrafish.

17. A solid support comprising one or a plurality of the polynucleotide of any one of claims 1 to 3, the gene of claim 4 or 5, the vector of claim 6 or 7, the polypeptide of claim 11, the antibody of claim 12 or 13 or the host cell of claim 8 in immobilized form.

18. The solid support of claim 17, wherein said solid support is a membrane, a glass-, polypropylene- or silicon-chip, are oligonucleotide conjugated beads or bead array, which is assembled on an optical filter substrate.

19. An in vitro method for identifying a polymorphism said method comprising the steps of:
(a) isolating a polynucleotide of any one claims 1 to 3 or the gene of claim 4 or 5 from a plurality of subgroups of individuals, wherein one subgroup has no prevalence for a CYP3A5 associated disease and at least one or more further subgroup(s) do have prevalence for a CYP3A5 associated disease; and
(b) identifying a polymorphism by comparing the nucleic acid sequence of said polynucleotide or said gene of said one subgroup having no prevalence for a CYP3A5 associated disease with said at least one or more further subgroup(s) having a prevalence CYP3A5 associated disease.

20. A method for identifying and obtaining a pro-drug or a drug capable of modulating the activity of a molecular variant of a CYP3A5 polypeptide comprising the steps of:
(a) contacting the polypeptide of claim 11, the solid support of claim 17 or 18, a cell expressing a molecular variant gene comprising a polynucleotide of any one of claims 1 to 3, the gene of claim 4 or 5 or the vector of claim 6 or 7 in the presence of components capable of providing a detectable signal in response to drug activity with a compound to be screened for pro-drug or drug activity; and
(c) detecting the presence or absence of a signal or increase or decrease of a signal generated from the pro-drug or the drug activity, wherein the absence, presence, increase or decrease of the signal is indicative for a putative pro-drug or drug.

21. A method for identifying and obtaining an inhibitor of the activity of a molecular variant of a CYP3A5 polypeptide comprising the steps of:
(d) contacting the protein of claim 11, the solid support of claim 17 or 18 or a cell expressing a molecular variant gene comprising a polynucleotide of any one of claims 1 to 3 or the gene of claim 4 or 5 or the vector of claim 6 or 7 in the presence of components capable of providing a detectable signal in response to drug activity with a compound to be screened for inhibiting activity; and
(e) detecting the presence or absence of a signal or increase or decrease of a signal generated from the inhibiting activity, wherein the absence or decrease of the signal is indicative for a putative inhibitor.

22. The method of claim 20 or 21, wherein said cell is a cell of claim 9, obtained by the method of claim 10 or can be obtained by the transgenic non-human animal of claim 15 or 16.

23. A method of identifying and obtaining a pro-drug or drug capable of modulating the activity of a molecular variant of a CYP3A5 polypeptide comprising the steps of:
(a) contacting the host cell of claim 8, the cell obtained by the method of claim 10, the polypeptide of claim 11 or the solid support of claim 17 or 18 with the first molecule known to be bound by a CYP3A5 polypeptide to form a first complex of said polypeptide and said first molecule;
(b) contacting said first complex with a compound to be screened, and
(c) measuring whether said compound displaces said first molecule from said first complex.

24. A method of identifying and obtaining an inhibitor capable of the activity of a molecular variant of a CYP3A5 polypeptide or its gene product comprising the steps of:
(a) contacting the host cell of claim 8, the cell obtained by the method of claim 10, the protein of claim 11 or the solid support of claim 17 or 18 with the first molecule known to be bound by a CYP3A5 polypeptide to form a first complex of said protein and said first molecule;
(b) contacting said first complex with a compound to be screened, and
(c) measuring whether said compound displaces said first molecule from said first complex.

25. The method of claim 23 or 24, wherein said measuring step comprises measuring the formation of a second complex of said protein and said compound.

26. The method of any one of claim 23 to 25, wherein said measuring step comprises measuring the amount of said first molecule that is not bound to said protein.

27. The method of any one of claims 23 to 26, wherein said first molecule is labeled.

28. A method for the production of a pharmaceutical composition comprising the steps of the method of any one of claims 20 to 27; and the further step of formulating the compound identified and obtained or a derivative thereof in a pharmaceutically acceptable form.

29. A method of diagnosing a disorder related to the presence of a molecular variant of a CYP3A5 gene or susceptibility to such a disorder comprising determining the presence of a polynucleotide of any one of claims 1 to 3 or the gene of claim 4 or 5 in a sample from a subject.

30. The method of claim 29 further comprising determining the presence of a polypeptide of claim 11 or the antibody of any one of claims 12 to 14.

31. A method of diagnosing a disorder related to the presence of a molecular variant of a CYP3A5 gene or susceptibility to such a disorder comprising determining the presence of a polypeptide of claim 11 or the antibody of any one of claims 12 to 14 in a sample from a subject.

32. The method of any one of claims 29 to 31, wherein said disorder is cancer or diseases including cardiovascular diseases, diabetes and AIDS.

33. The method of any one of claims 29 to 32 comprising PCR, ligase chain reaction, restriction digestion, direct sequencing, nucleic acid amplification techniques, hybridization techniques mass spectroscopy or immunoassays.

34. A method of detection of the polynucleotide of any one of claims 1 to 3 or the gene of claim 4 or 5 in a sample comprising the steps of
(a) contacting the solid support of claim 17 or 18 with the sample under conditions allowing interaction of the polynucleotide of claim 1 to 3 or the gene of claim 4 or 5 with the immobilized targets on a solid support and;
(b) determining the binding of said polynucleotide or said gene to said immobilized targets on a solid support.

35. An in vitro method for diagnosing a disease comprising the steps of the method of claim 34, wherein binding of said polynucleotide or gene to said immobilized targets on said solid support is indicative for the presence or the absence of said disease or a prevalence for said disease.

36. A diagnostic composition comprising the polynucleotide of any one of claims 1 to 4, the gene of claim 3 or 4, the vector of claim 6 or 7, the polypeptide of claim 11 or the antibody of claim 12 or 13.

37. A pharmaceutical composition comprising the polynucleotide of any one of claims 1 to 3, the gene of claim 4 or 5, the vector of claim 6 or 7, the polypeptide of claim 11 or the antibody of claim 12 or 13.

38. Use of the polynucleotide of any one of claims 1 to 3, a polynucleotide comprising SEQ ID No: 104, a polynucleotide encoding a polypeptide comprising SEQ ID No: 145, the gene of claim 4 or 5, the vector of claim 6 or 7, the polypeptide of claim 11, a polypeptide comprising SEQ ID No: 145 or the antibody of claim 12 or 13 for the preparation of a diagnostic composition for diagnosing a disease.

39. Use of the polynucleotide of any one of claims 1 to 3, a polynucleotide comprising SEQ ID No: 104, a polynucleotide encoding a polypeptide comprising SEQ ID No: 145, the gene of claim 4 or 5, the vector of claim 6 or 7, the polypeptide of claim 11, a polypeptide comprising SEQ ID No: 145 or the antibody of claim 12 or 13 for the preparation of a pharmaceutical composition for treating a disease.

40. Use of a polynucleotide selected from the group consisting of:
(a) a polynucleotide having the nucleic acid sequence of SEQ ID NO: 82, 88, 104 or 112;
(b) a polynucleotide encoding a polypeptide having the amino acid sequence of SEQ ID No: 127, 132, 141 or 145;
(c) a polynucleotide capable of hybridizing to a CYP3A5 gene, wherein said polynucleotide is having at least one additional nucleotide at a position corresponding to position 3709/3710 or 27131/27132 of the CYP3A5 gene (Accession No: AF280107.1, wherein position 166220 has been numbered +1 and position 174832 has been numbered +8613, and Accession No: AC005020.2, wherein position 27341 has been numbered +8614) or a nucleotide exchange at a position corresponding to position 7303 or 27289 of the CYP3A5 gene (Accession No: AF280107.1, wherein position 166220 has been numbered +1 and position 174832 has been numbered +8613, and Accession No: AC005020.2, wherein position 27341 has been numbered +8614);
(d) a polynucleotide capable of hybridizing to a CYP3A5 gene, wherein said polynucleotide is having an additional G nucleotide at a position corresponding to position 3709/3710 of the CYP3A5 gene (Accession No: AF280107.1, wherein position 166220 has been numbered +1 and position 174832 has been numbered +8613, and Accession No: AC005020.2, wherein position 27341 has been numbered +8614), an additional T nucleotide at a position corresponding to position 27131/27132 of the CYP3A5 gene (Accession No: AF280107.1, wherein position 166220 has been numbered +1 and position 174832 has been numbered +8613, and Accession No: AC005020.2, wherein position 27341 has been numbered +8614), or an A at a position corresponding to position 7303 or 27289 of the CYP3A5 gene (Accession No: AF280107.1, wherein position 166220 has been numbered +1 and position 174832 has been numbered +8613, and Accession No: AC005020.2, wherein position 27341 has been numbered +8614);
for the preparation of a diagnostic composition for diagnosing a disease in a subject having a genome comprising a variant allele of the CYP3A5 gene, wherein said allele is having an A at a position corresponding to position 6986 of the CYP3A5 gene (Accession No: AF280107.1, wherein position 166220 has been numbered +1 and position 174832 has been numbered +8613, and Accession No: AC005020.2, wherein position 27341 has been numbered +8614).

41. Use of a polynucleotide comprising a polynucleotide having an A at a position corresponding to position 14690 of the CYP3A5 gene (Accession No: AF280107.1, wherein position 166220 has been numbered +1 and position 174832 has been numbered +8613, and Accession No: AC005020.2, wherein position 27341 has been numbered +8614) for the preparation of a diagnostic composition for diagnosing a disease in a subject having a genome comprising a variant allele of the CYP3A5 gene, wherein said allele is having an A at a position corresponding to position 6986 of the CYP3A5 gene (Accession No: AF280107.1, wherein position 166220 has been numbered +1 and position 174832 has been numbered +8613, and Accession No: AC05020.2, wherein position 27341 has been numbered +8614).

42. The use of claim 40 or 41, wherein said subject is an African American.

43. The use of any one of claims 38 to 41, wherein said disease is cancer or diseases including cardiovascular diseases, diabetes and AIDS.

44. A diagnostic kit for detection of a single nucleotide polymorphism comprising the polynucleotide of any one of claims 1 to 3, the gene of claim 4 or 5, the vector of claim 6 or 7, the polypeptide of claim 11, the antibody of claim 12 or 13, the host cell of claim 8, the transgenic non-human animal of claim 15 or 16 or the solid support of claim 17 or 18.


SEQUENCE LISTING (239 sequences)

SEQ ID NO: 1 - DNA, 21 bp, Homo sapiens
acaggcacagaaacccacaag

SEQ ID NO: 2 - DNA, 19 bp, Homo sapiens
atcgccacttgccttcttc

SEQ ID NO: 3 - DNA, 19 bp, Homo sapiens
ccctgcttcggcttgtgca

SEQ ID NO: 4 - DNA, 24 bp, Homo sapiens
cacagcctgctttatttgtcatga

SEQ ID NO: 5 - DNA, 21 bp, Homo sapiens
gatccttggtaggacaagcct

SEQ ID NO: 6 - DNA, 24 bp, Homo sapiens
caagcactgatttggtcacttcct

SEQ ID NO: 7 - DNA, 22 bp, Homo sapiens
gggatgggaccgtaagtggaac

SEQ ID NO: 8 - DNA, 26 bp, Homo sapiens
taatcacattggagttctgacaaatg

SEQ ID NO: 9 - DNA, 30 bp, Homo sapiens
aaaaacctcttacaaaagtatcatcggata

SEQ ID NO: 10 - DNA, 27 bp, Homo sapiens
cctactaggtctctgacttggaaccat

SEQ ID NO: 11 - DNA, 21 bp, Homo sapiens
gccgagacgcaccattacact

SEQ ID NO: 12 - DNA, 21 bp, Homo sapiens
cacccatcccttcccactcat

SEQ ID NO: 13 - DNA, 28 bp, Homo sapiens
tgatggttccaagtcagagacctagtag

SEQ ID NO: 14 - DNA, 35 bp, Homo sapiens
aattgtagacatctttctcttaagttaattcccag

SEQ ID NO: 15 - DNA, 25 bp, Homo sapiens
tctgcatgccaacagtgaacaatct

SEQ ID NO: 16 - DNA, 19 bp, Homo sapiens
ggcacgcaccagcatgtcc

SEQ ID NO: 17 - DNA, 19 bp, Homo sapiens
ctggctgagtgccgtggct

SEQ ID NO: 18 - DNA, 25 bp, Homo sapiens
tgagcgcttcatgtattctggctat

SEQ ID NO: 19 - DNA, 31 bp, Homo sapiens
aaatattttcaaagtcacactctgacaacag

SEQ ID NO: 20 - DNA, 29 bp, Homo sapiens
taacaggatctcatgcttttttcatggct

SEQ ID NO: 21 - DNA, 30 bp, Homo sapiens
cactccaatattcacaatagccactattca

SEQ ID NO: 22 - DNA, 24 bp, Homo sapiens
actcctacgtatccttccaagccc

SEQ ID NO: 23 - DNA, 28 bp, Homo sapiens
gctaagggaaacaggcatagaaacttac

SEQ ID NO: 24 - DNA, 18 bp, Homo sapiens
ggagcttccctgccctgc

SEQ ID NO: 25 - DNA, 21 bp, Homo sapiens
tccttctccagcacataaatc

SEQ ID NO: 26 - DNA, 21 bp, Homo sapiens
aaattagaaggtggatgggag

SEQ ID NO: 27 - DNA, 21 bp, Homo sapiens
gagtaactcaccagccctctg

SEQ ID NO: 28 - DNA, 21 bp, Homo sapiens
aaacctcagaactccctccca

SEQ ID NO: 29 - DNA, 23 bp, Homo sapiens
gacatctctgaatagcttccttc

SEQ ID NO: 30 - DNA, 21 bp, Homo sapiens
gcacatagtttataacggcaa

SEQ ID NO: 31 - DNA, 23 bp, Homo sapiens
agaacctaaggttgctgtgtgtc

SEQ ID NO: 32 - DNA, 21 bp, Homo sapiens
tgcaagatgttaccactgggc

SEQ ID NO: 33 - DNA, 20 bp, Homo sapiens
cgccccacatacactcagaa

SEQ ID NO: 34 - DNA, 21 bp, Homo sapiens
agaccatttttaggaagctcg

SEQ ID NO: 35 - DNA, 22 bp, Homo sapiens
caaggggtagtccactgagttc

SEQ ID NO: 36 - DNA, 18 bp, Homo sapiens
ctctttggagttgcagcg

SEQ ID NO: 37 - DNA, 21 bp, Homo sapiens
aggtgagtctaactcagcttg

SEQ ID NO: 38 - DNA, 21 bp, Homo sapiens
gacagctaaagtgtgtgaggg

SEQ ID NO: 39 - DNA, 21 bp, Homo sapiens
aatgggttccagttgagaatc

SEQ ID NO: 40 - DNA, 21 bp, Homo sapiens
attgttgtgcctgatttcaag

SEQ ID NO: 41 - DNA, 19 bp, Homo sapiens
agaagccatagggaggttg

SEQ ID NO: 42 - DNA, 20 bp, Homo sapiens
gactgtcctccaagcattct

SEQ ID NO: 43 - DNA, 21 bp, Homo sapiens
gatgccatgatgaggagtgtg

SEQ ID NO: 44 - DNA, 19 bp, Homo sapiens
accagggccagcaatattg

SEQ ID NO: 45 - DNA, 25 bp, Homo sapiens
aaatacttcacgaatactatgatca

SEQ ID NO: 46 - DNA, 24 bp, Homo sapiens
cagggacataattgattatctttg

SEQ ID NO: 47 - DNA, 19 bp, Homo sapiens
tactggttgggaggtggag

SEQ ID NO: 48 - DNA, 23 bp, Homo sapiens
catgatgttcttaatgctacagg

SEQ ID NO: 49 - DNA, 25 bp, Homo sapiens
gaagagttcaagatacatggtgtta

SEQ ID NO: 50 - DNA, 23 bp, Homo sapiens
tgcacaacactctacacagactc

SEQ ID NO: 51 - DNA, 11 bp, Homo sapiens
tgggcttgcaa

SEQ ID NO: 52 - DNA, 11 bp, Homo sapiens
tgggcgtgcaa

SEQ ID NO: 53 - DNA, 11 bp, Homo sapiens
gcatgggtaaa

SEQ ID NO: 54 - DNA, 11 bp, Homo sapiens
gcatgagtaaa

SEQ ID NO: 55 - DNA, 11 bp, Homo sapiens
ggggtgtgtgc

SEQ ID NO: 56 - DNA, 11 bp, Homo sapiens
ggggtatgtgc

SEQ ID NO: 57 - DNA, 11 bp, Homo sapiens
tgtgcgattct

SEQ ID NO: 58 - DNA, 11 bp, Homo sapiens
tgtgcaattct

SEQ ID NO: 59 - DNA, 11 bp, Homo sapiens
gccccacctcc

SEQ ID NO: 60 - DNA, 11 bp, Homo sapiens
gccccgcctcc

SEQ ID NO: 61 - DNA, 11 bp, Homo sapiens
ctcacactggg

SEQ ID NO: 62 - DNA, 11 bp, Homo sapiens
ctcacgctggg

SEQ ID NO: 63 - DNA, 11 bp, Homo sapiens
gagacgcacca

SEQ ID NO: 64 - DNA, 11 bp, Homo sapiens
gagacacacca

SEQ ID NO: 65 - DNA, 11 bp, Homo sapiens
tgtgtgtggga

SEQ ID NO: 66 - DNA, 11 bp, Homo sapiens
tgtgtatggga

SEQ ID NO: 67 - DNA, 11 bp, Homo sapiens
atccatgtata

SEQ ID NO: 68 - DNA, 11 bp, Homo sapiens
atccacgtata

SEQ ID NO: 69 - DNA, 11 bp, Homo sapiens
catcttacccc

SEQ ID NO: 70 - DNA, 11 bp, Homo sapiens
catctcacccc

SEQ ID NO: 71 - DNA, 11 bp, Homo sapiens
tctattgctat

SEQ ID NO: 72 - DNA, 11 bp, Homo sapiens
tctatagctat

SEQ ID NO: 73 - DNA, 11 bp, Homo sapiens
ggcagggaagc

SEQ ID NO: 74 - DNA, 11 bp, Homo sapiens
ggcagagaagc

SEQ ID NO: 75 - DNA, 11 bp, Homo sapiens
ccaggcaaaca

SEQ ID NO: 76 - DNA, 11 bp, Homo sapiens
ccaggtaaaca

SEQ ID NO: 77 - DNA, 11 bp, Homo sapiens
tcaaggagaag

SEQ ID NO: 78 - DNA, 11 bp, Homo sapiens
tcaagaagtag

SEQ ID NO: 79 - DNA, 11 bp, Homo sapiens
gtacacatgga

SEQ ID NO: 80 - DNA, 11 bp, Homo sapiens
gtacatatgga

SEQ ID NO: 81 - DNA, 10 bp, Homo sapiens
catggacttt

SEQ ID NO: 82 - DNA, 11 bp, Homo sapiens
catgggacttt

SEQ ID NO: 83 - DNA, 11 bp, Homo sapiens
gatagcaggcc

SEQ ID NO: 84 - DNA, 11 bp, Homo sapiens
gatagtaggcc

SEQ ID NO: 85 - DNA, 11 bp, Homo sapiens
agaatcgggct

SEQ ID NO: 86 - DNA, 11 bp, Homo sapiens
agaatagggct

SEQ ID NO: 87 - DNA, 11 bp, Homo sapiens
ttattctgtct

SEQ ID NO: 88 - DNA, 11 bp, Homo sapiens
ttattatgtct

SEQ ID NO: 89 - DNA, 11 bp, Homo sapiens
gtacaggaagg

SEQ ID NO: 90 - DNA, 11 bp, Homo sapiens
cctaaaaaatg

SEQ ID NO: 91 - DNA, 11 bp, Homo sapiens
tcttttatctt

SEQ ID NO: 92 - DNA, 11 bp, Homo sapiens
tctttgatctt

SEQ ID NO: 93 - DNA, 11 bp, Homo sapiens
gagtctgcaca

SEQ ID NO: 94 - DNA, 11 bp, Homo sapiens
gagtccgcaca

SEQ ID NO: 95 - DNA, 11 bp, Homo sapiens
gaagtcaagaa

SEQ ID NO: 96 - DNA, 11 bp, Homo sapiens
agtcgtcaaga

SEQ ID NO: 97 - DNA, 11 bp, Homo sapiens
aggaagtattc

SEQ ID NO: 98 - DNA, 11 bp, Homo sapiens
aggaattattc

SEQ ID NO: 99 - DNA, 11 bp, Homo sapiens
agagagcttca

SEQ ID NO: 100 - DNA, 11 bp, Homo sapiens
agagaacttca

SEQ ID NO: 101 - DNA, 11 bp, Homo sapiens
cttcaatagta

SEQ ID NO: 102 - DNA, 11 bp, Homo sapiens
cttcagtagta

SEQ ID NO: 103 - DNA, 11 bp, Homo sapiens
tccaacttatg

SEQ ID NO: 104 - DNA, 11 bp, Homo sapiens
tccaaattatg

SEQ ID NO: 105 - DNA, 11 bp, Homo sapiens
cgaaactacat

SEQ ID NO: 106 - DNA, 11 bp, Homo sapiens
cgaaattacat

SEQ ID NO: 107 - DNA, 11 bp, Homo sapiens
aaggatttcta

SEQ ID NO: 108 - DNA, 11 bp, Homo sapiens
aaggacttcta

SEQ ID NO: 109 - DNA, 11 bp, Homo sapiens
agctccgttgt

SEQ ID NO: 110 - DNA, 11 bp, Homo sapiens
agctctgttgt

SEQ ID NO: 111 - DNA, 10 bp, Homo sapiens
cacctaccta

SEQ ID NO: 112 - DNA, 11 bp, Homo sapiens
caccttaccta

SEQ ID NO: 113 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcgtgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaatatgaactcaaaggaggtaagcaaaggggtgtgtgcgattctttgctactggctgcagctgcagccccgcctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 114 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcttgcaaggatgtgtaggagtcatctagggggcacaagtacactccaggcagagggaattgcatgagtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaatatgaactcaaaggaggtaagcaaaggggtgtgtgcgattctttgctactggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 115 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcttgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaatatgaactcaaaggaggtaagcaaaggggtatgtgcgattctttgctactggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 116 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcttgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaatatgaactcaaaggaggtaagcaaaggggtgtgtgcaattctttgctactggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 117 - DNA, 775 bp, Homo sapiens
ggagtgacctgattttccaggtgctgtctgtcacccctttctttgactaggaaagggaactccttgaccccttgcgcttctcaagtgaggcaatgcctcgccctgcttcggcttgtgcacagcacgctgcacccactgtcctgcacccactgtctggcactccctagtgagatgaacccggtacctcagatgaaaatgcagaaatcacccgtcttctgtgtcactcacgctgggagctgtagaccggagctgttcctattcggccatcttggctccaccccccgagttttggcttttaattgaaagtgtattgatgtgggaaggagataatgccatgcatttatgagcacatattagagggtctgagacaatgcatgtgataaaaggtcacctaaggaagaaaaaaagaacaagggaagacactggaaagaacgtgatgctgggagtccctgggccaccaaagtctggagaaaagtggtaaccacaaggctcccagcctagtttcactgaagacctcgacactaggtgacttatgggatccttggtaggacaagccttgaaaagttttacaatagcaaatgtggacgttgtcagaaccaaatgatgtcacgtgtgtatttgtgtgtgtgtgtcagtgtgtgtgtttaaaaatcatgacaaataaagcaggctgtgaagaggggattcccatgctcgtgtgcctgataacacaactatcacaaacgctttgcgaaaccacaagtttgcacaaaggcaatcccaaccttacacaaaa

SEQ ID NO: 118 - DNA, 1110 bp, Homo sapiens
taatgaaaataagaattattttgatggctctaacagtgacatttatatcatctgttttatctggagcattctataataagtttatattaagcaaatcaataaaaacctcttacaaaagtatcatcggatactttcctgaacattaaggagaaatctatagaactgaatgaatgagaaccaacaagtaaatatatgtgatcattgtaaccattgttggtgtggggcatttgtcagaactccaatgtgattattaacataggtgagaattaatccactgtgactttgcccattgcttagaaagaacattcatagtttaattatgccttttttgaccaagcacagtggctcatgcctgtaatcccagcactttgggaggccgaggtgggtggatcacctgaggtcaggagttcgagaccagcctgaccaacatggtgaaaccccatctctactaaaaatacaaaaattagctaggtgtggtggtatgcacctataatctcagctacccaggaggctgaggcaggagaatcacttgaacctggaggcagaggttgcagtgagccgagacacaccattacactccagcctgggtgacagagtgagattccatctcaaaaaaaaaaaaaaaaaattatgcctttttgaagcacatacattttataacatacaactgaatcccttattatattattagttttgatttaatgttttcaaaccatctcccctgatatttctgggagatgggaaacatgttttcttacacctcttgcattccattctcaactcccaactgtcttactgcaatgaacacttaataagaaacagtcaattggtcaattgattgggcaacaggctaaacacactcattccttgtctgttcccacttctttctttactttcccttcctgagtaacttatcctaaagtcattaggtgggtggcagccagatggtggccacacattaaggtagaaaagagagtgtcatgatggttccaagtcagagacctagtagggtgaggatcaagtaggtgttcacgtggagaaacagcccggcctgtgtgtgggagtccaagcaagcagagaaaatgtcgacacagaggggtggcctgaaaa

SEQ ID NO: 119 - DNA, 837 bp, Homo sapiens
caaaaattagctaggtgtggtggtatgcacctataatctcagctacccaggaggctgaggcaggagaatcacttgaacctggaggcagaggttgcagtgagccgagacgcaccattacactccagcctgggtgacagagtgagattccatctcaaaaaaaaaaaaaaaaaattatgcctttttgaagcacatacattttataacatacaactgaatcccttattatattattagttttgatttaatgttttcaaaccatctcccctgatatttctgggagatgggaaacatgttttcttacacctcttgcattccattctcaactcccaactgtcttactgcaatgaacacttaataagaaacagtcaattggtcaattgattgggcaacaggctaaacacactcattccttgtctgttcccacttctttctttactttcccttcctgagtaacttatcctaaagtcattaggtgggtggcagccagatggtggccacacattaaggtagaaaagagagtgtcatgatggttccaagtcagagacctagtagggtgaggatcaagtaggtgttcacgtggagaaacagcccggcctgtgtatgggagtccaagcaagcagagaaaatgtcgacacagaggggtggcctgaaaaagcagccagagcctaaacagggcatggagaacatatttagggcatgaggtgaggagggcatccatgagtgggaagggatgggtgaggtttcactacataaaggggattgatgaaataagtaaataaagtatactggaagccaggtgtgtcacttttgcagaaaagagtcatggattcagaaagg

SEQ ID NO: 120 - DNA, 1197 bp, Homo sapiens
ttctttctttactttcccttcctgagtaacttatcctaaagtcattaggtgggtggcagccagatggtggccacacattaaggtagaaaagagagtgtcatgatggttccaagtcagagacctagtagggtgaggatcaagtaggtgttcacgtggagaaacagcccggcctgtgtgtgggagtccaagcaagcagagaaaatgtcgacacagaggggtggcctgaaaaagcagccagagcctaaacagggcatggagaacatatttagggcatgaggtgaggagggcatccatgagtgggaagggatgggtgaggtttcactacataaaggggattgatgaaataagtaaataaagtatactggaagccaggtgtgtcacttttgcagaaaagagtcatggattcagaaagggagaaaactagcaggaatcctatgaaattagattaaaatggatgtatccacgtatattcatacccttctagatagataaatggttagataggtgataaaaagataacaagaggacaagataattagatagacataaatgtatgtatgtgtttgtgtgtgtgtacaaaaaaacatatactccctacttctctccactgatagggctaggtaacaatggcatttcaatagcaatgagcacacttagtggccagatcttggcttattaataccattttccactgaaaggaaccagagctttttagagaaatggctgattccagggccaggattaagaatgttcaagataagcctaggatacattttgtgccaggaagcaagaagatgttcaaatgatttccaagtaatgtttggaaatgatatttgaaaatgatttccaaatgatatttccaaatgatttccaaatgatatatggaaacacttaaagactccactaaagaactattagatctgataaacaaattcagtaatgttgctggatacaaaatcaacatacaaaaaccagtagcatttctgcatgccaacagtgaacaatctggcaaaaataaaaaatgtaatcccatttacaataaccccaaataaaactaaatacctgggaattaacttaagagaaagatgtctacaattaatattgtaaaacactgatgaaggaaattgaagaagacacaaaaaagaaggatattccatgtttatatattgtaagcattaatattgttaaaaatgtcca

SEQ ID NO: 121 - DNA, 817 bp, Homo sapiens
caaagcaaaaatggacaaatggatcagatcaagttaaaaagcttctgtaccacaaagaaagcaatcaacaaagtgaagacacaaaccacagaatgggagaaaatattttcaaagtcacactctgacaacagattaatagccagaatacatgaagcgctcaaacaactctgtaaggaaaaatctaataatccaatcaaaaaatgggcaaaatttgaatagacatttttcaaaagaagacatacaaatgccacataggcatatgataaggtgctcaacatcactggtcattagagaaatgcaaatcaaaaccacaatgagatatcatctcaccccagctaaaatggtttttatccaaaagacaggcaacaacaaatgccagcgagaatgtggagaaaagggaacccttgtacactgttggtgtaaattagtgcaaccactatagagaacaatttggaggttcctcaaaacattaaaattaacattaaatagagctaccacaatatccagaaatccccatgctgggtatatacctggaagaaaggaaatcatatattgaagagataacatcactccaatattcacaatagccactattcacaaatgccaagatttggaagcaacctaagtgtccatcaacagatgaatggataaagaaagtactccaattatacacaatggagcacaattcagccatgaaaaaagcatgagatcctgttatctgtaataatatggatggaactggaggtcatcatgttaagtgaaataagccaggcacagaaacacagatattgcaagttctcacatacttgtgggatct

SEQ ID NO: 122 - DNA, 1126 bp, Homo sapiens
cattaaaattaacattaaatagagctaccacaatatccagaaatccccatgctgggtatatacctggaagaaaggaaatcatatattgaagagataacatcactccaatattcacaatagccactattcacaaatgccaagatttggaagcaacctaagtgtccatcaacagatgaatggataaagaaagtactccaattatacacaatggagcacaattcagccatgaaaaaagcatgagatcctgttatctgtaataatatggatggaactggaggtcatcatgttaagtgaaataagccaggcacagaaacacagatattgcaagttctcacatacttgtgggatctacaaatcaaaacaactgagctaatgtctgggccttagtcagtgttgtacccaagtactgggagcacagcttttaaaatacatcatgaatgctttaatacaggaatgaatagatgagaggcacaaactggttgggtgttcttctgatacacagtatcttccttgacagattcagtacaactctcaacaggtaagtctcttcatgttatgttaccttatgaggaattaagtggcagaacatgatttctattattttcctttgcagaacaagaccaactttattagttgggacacagtgtggctgcatttgagtcccaagcaaccattagtctatagctatcaccacagagtcagaggggatgagacgcccagcaatctcacccaagacaactccaccaacattcctggttacccaccatgtgtacagtaccctgctaggaaccagggtcatgaaagtaaataataccagactgtgcccttgaggagctcacctctgctaagggaaacaggcatagaaacttacaatggtggtagagagaaaagaggacaataggactgtgtgagggggataggaggcacccagaggaggaaatggttacatttgtgtgaggaggttggtaaggaaaaattttagcagaaggggtctgtctggctgggcttggaaggatacgtaggagtcatctagagggcacaggtacactccaggcagagggaatttcgtgggtaaagatgtgtaggtgtggcttgtgaggatggatttcaattattctagaatgaa

SEQ ID NO: 123 - DNA, 624 bp, Homo sapiens
gattaagcttttcatgattcctcatagaacatgaactcaaaagaggtcagcaaaggggtgtgtgcgattctttgctattggctgcagctatagccctgcctccttctccagcacataaatctttcagcagcttggctgaagactgctgtgcagggcagagaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcaga

SEQ ID NO: 124 - DNA, 624 bp, Homo sapiens
gattaagcttttcatgattcctcatagaacatgaactcaaaagaggtcagcaaaggggtgtgtgcgattctttgctattggctgcagctatagccctgcctccttctccagcacataaatctttcagcagcttggctgaagactgctgtgcagggcagggaagctccaggtaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcaga

SEQ ID NO: 125 - DNA, 621 bp, Homo sapiens
gattaagcttttcatgattcctcatagaacatgaactcaaaagaggtcagcaaaggggtgtgtgcgattctttgctattggctgcagctatagccctgcctccttctccagcacataaatctttcagcagcttggctgaagactgctgtgcagggcagggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcaga

SEQ ID NO: 126 - DNA, 465 bp, Homo sapiens
aggaaaggacctgatgagtgaatgcaattactgatgttggagttgctgttattatttatcgtgtacatattacctccctctcttgaccattccagttcctgagtaactcaccagccctctgatctataaagtcacaatccctgtgacctgatttctgtttcactttgtagatatgggacccgtacatatgggactttttaagagactgggaattccagggcccacacctctgcctttgttgggaaatgttttgtcctatcgtcaggtgagttgcttgagcttcctcttttgcttcttatggttgcaaacatcagcttagttccatcagtaaaaatgcccctccttgggagggagttctgaggtttcacattttcagaaatggtgggactgggtgcagtggatcatgcctgtaatctcagcctctgtgaggccaagactggcaaattgcttgagcccaggagtttg

SEQ ID NO: 127 - protein, 33 aa, Homo sapiens
MDLIPNLAVETWLLLAVSLVLLYLYGTRTYGTF

SEQ ID NO: 128 - DNA, 602 bp, Homo sapiens
catagacaagggtgagtccttcagtacttagagaaaattcaagagtgactttaaattccccacttcaaatatattctctgttttcttgtctttcccttaagacatctctgaatagcttccttcaactgccagtgaaagatagtaggcctgatttcattggacgcaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaata

SEQ ID NO: 129 - DNA, 594 bp, Homo sapiens
cagtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatagggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctg

SEQ ID NO: 130 - DNA, 594 bp, Homo sapiens
cagtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctctgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctg

SEQ ID NO: 131 - DNA, 594 bp, Homo sapiens
cagtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattatgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctg

SEQ ID NO: 132 - protein, 60 aa, Homo sapiens
MWGTYEGQLPVLAITDPDVIRTVLVKECYYVFTNRRSLGPVGFMKSAISLAEDEEWKRIR

SEQ ID NO: 133 - DNA, 601 bp, Homo sapiens
cagtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacctaaaaaatgaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctg

SEQ ID NO: 134 - DNA, 603 bp, Homo sapiens
agcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtctttgatctttatgtacagaaaagacatcacaaaattcattacaaaatgtcacttactgctccatgctggagaaagccatatccttctgggacttgagtctgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaactt

SEQ ID NO: 135 - DNA, 603 bp, Homo sapiens
agcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtccgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaactt

SEQ ID NO: 136 - DNA, 626 bp, Homo sapiens
attggacatgatagctagatttgtttcaggaaaacatcctgctttccaaggatttagatgaatgtttttgttcactggtgactcaggtaacacgtcttcaagaagccatagggaggttgagggagggaagtcgtcaagaagggaggttgaggactgcacttttgatttacttctgacttcacgagtcactttctgccaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaagtattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacata

SEQ ID NO: 137 - DNA, 623 bp, Homo sapiens
attggacatgatagctagatttgtttcaggaaaacatcctgctttccaaggatttagatgaatgtttttgttcactggtgactcaggtaacacgtcttcaagaagccatagggaggttgagggagggaagtcaagaagggaggttgaggactgcacttttgatttacttctgacttcacgagtcactttctgccaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaattattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacata

SEQ ID NO: 138 - DNA, 826 bp, Homo sapiens
agataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcgtcatcctggccctgtataagatacatatcagaatgaaaaccactcccagtgtgactttgaattgcttttccattttttcttcttgggattagagaacttcacttagatttcatctaagctgtgatgttgtacgttgacctgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagc

SEQ ID NO: 139 - DNA, 795 bp, Homo sapiens
gtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcagtagtactgcatggactcagttgagagttaattcaaaatctcagattatccaattctgtttctttccttccaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaactacatccatccacagtcccagcacaagacaaagataatcaattatgtccctgtgggcatttttctacgcctatatagatttttaaaaattagaatggtatcactttttatttggtttgaattgctgcttacttgatttaacaggaaactatc

SEQ ID NO: 140 - DNA, 796 bp, Homo sapiens
gtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcaatagtactgcatggactcagttgagagttaattcaaaatctcagattatccaattctgtttctttccttccaggcaccaccttacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaactacatccatccacagtcccagcacaagacaaagataatcaattatgtccctgtgggcatttttctacgcctatatagatttttaaaaattagaatggtatcactttttatttggtttgaattgctgcttacttgatttaacaggaaactatc

SEQ ID NO: 141 - protein, 59 aa, Homo sapiens
ALSDLELAAQSIIFIFAGYETTSSVLSFTLYELATHPDVQQKLQKEIDAVLPNKAPPYL

SEQ ID NO: 142 - DNA, 795 bp, Homo sapiens
gtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcaatagtactgcatggactcagttgagagttaattcaaaatctcagattatccaattctgtttctttccttccaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaattacatccatccacagtcccagcacaagacaaagataatcaattatgtccctgtgggcatttttctacgcctatatagatttttaaaaattagaatggtatcactttttatttggtttgaattgctgcttacttgatttaacaggaaactatc

SEQ ID NO: 143 - DNA, 616 bp, Homo sapiens
caggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgtagatccccttgaaattagacacgcaaggacttcttcaaccagaaaaacccattgttctaaaggtggattcaagagatggaaccctaagtggagaatgagttattctaaggacttctactttggtcttcaagaaagctgtgccccagaacaccagagatttcaacttagtcaataaaaccttgaaataaagatgggcttaatctaatgtactgcatgagtagttggtgattttgtacattcattgagctctcccagagtctgtgtagagtgttgtgcattatgtagtataaaggaggtgaccaggtaagtgacagataggtagactcagcttctctgcttctcataggactacctctacccacctctagttagcatta

SEQ ID NO: 144 - DNA, 1508 bp, Homo sapiens
ccatggctctgttattagcagtttttctggtgctcctctatctatatgggacccgtacacatggactttttaagagactgggaattccagggcccacacctctgcctttgttgggaaatgttttgtcctatcgtcagggtctctggaaatttgacacagagtgctataaaaagtatggaaaaatgtggggaacgtatgaaggtcaactccctgtgctggccatcacagatcccgacgtgatcagaacagtgctagtgaaagaatgttattctgtcttcacaaatcgaaggtctttaggcccagtgggatttatgaaaagtgccatctctttagctgaggatgaagaatggaagagaatacggtcattgctgtctccaaccttcaccagcggaaaactcaaggagatgttccccatcattgcccagtatggagatgtattggtgagaaacttgaggcgggaagcagagaaaggcaagcctgtcaccttgaaagacatctttggggcctacagcatggatgtgattactggcacatcatttggagtgaacatcgactctctcaacaatccacaagacccctttgtggagagcactaagaagttcctaaaatttggtttcttagatccattatttctctcaataatactctttccattccttaccccagtttttgaagcattaaatgtctctctgtttccaaaagataccataaattttttaagtaaatctgtaaacagaatgaagaaaagtcgcctcaacgacaaacaaaagcaccgactagatttccttcagctgatgattgactcccagaattcgaaagaaactgagtcccacaaagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacagatccccttgaaattagacacgcaaggacttcttcaaccagaaaaacccattgttctaaaggtggattcaagagatggaaccctaagtggagaacatcaccatcaccatcactgagatctgcag

SEQ ID NO: 145 - protein, 60 aa, Homo sapiens
PVAIRLERTCKKDVEINGVFIPKGSMVVIPNYALHHDPKYWTEPEEFRPERFSKKKDSID

SEQ ID NO: 146 - DNA, 11 bp, Homo sapiens
tttcagtatct

SEQ ID NO: 147 - DNA, 11 bp, Homo sapiens
tttcaatatct

SEQ ID NO: 148 - DNA, 11 bp, Homo sapiens
tttaatagaag

SEQ ID NO: 149 - DNA, 11 bp, Homo sapiens
tttaacagaag

SEQ ID NO: 150 - DNA, 11 bp, Homo sapiens
attccccatag

SEQ ID NO: 151 - DNA, 11 bp, Homo sapiens
attcctcatag

SEQ ID NO: 152 - DNA, 11 bp, Homo sapiens
tagaatatgaa

SEQ ID NO: 153 - DNA, 11 bp, Homo sapiens
tagaacatgaa

SEQ ID NO: 154 - DNA, 11 bp, Homo sapiens
gtaacttatcc

SEQ ID NO: 155 - DNA, 11 bp, Homo sapiens
gtaacgtatcc

SEQ ID NO: 156 - DNA, 11 bp, Homo sapiens
ttcacgtggag

SEQ ID NO: 157 - DNA, 11 bp, Homo sapiens
ttcacatggag

SEQ ID NO: 158 - DNA, 11 bp, Homo sapiens
tggacgcaact

SEQ ID NO: 159 - DNA, 11 bp, Homo sapiens
tggacacaact

SEQ ID NO: 160 - DNA, 11 bp, Homo sapiens
gaggataatta

SEQ ID NO: 161 - DNA, 11 bp, Homo sapiens
gaggaaaatta

SEQ ID NO: 162 - DNA, 11 bp, Homo sapiens
catcattgccc

SEQ ID NO: 163 - DNA, 11 bp, Homo sapiens
catcactgccc

SEQ ID NO: 164 - DNA, 11 bp, Homo sapiens
cagtcgcactg

SEQ ID NO: 165 - DNA, 11 bp, Homo sapiens
cagtcacactg

SEQ ID NO: 166 - DNA, 11 bp, Homo sapiens
actaagaagtt

SEQ ID NO: 167 - DNA, 11 bp, Homo sapiens
actaaaaagtt

SEQ ID NO: 168 - DNA, 11 bp, Homo sapiens
gatccattatt

SEQ ID NO: 169 - DNA, 11 bp, Homo sapiens
gatccgttatt

SEQ ID NO: 170 - DNA, 11 bp, Homo sapiens
caattccattg

SEQ ID NO: 171 - DNA, 11 bp, Homo sapiens
caatttcattg

SEQ ID NO: 172 - DNA, 11 bp, Homo sapiens
tgtcaatctag

SEQ ID NO: 173 - DNA, 11 bp, Homo sapiens
tgtcagtctag

SEQ ID NO: 174 - DNA, 11 bp, Homo sapiens
ttgtttgtttt

SEQ ID NO: 175 - DNA, 11 bp, Homo sapiens
ttgttcgtttt

SEQ ID NO: 176 - DNA, 11 bp, Homo sapiens
aaataaagaag

SEQ ID NO: 177 - DNA, 11 bp, Homo sapiens
aaatacagaag

SEQ ID NO: 178 - DNA, 11 bp, Homo sapiens
tttgcgtcatc

SEQ ID NO: 179 - DNA, 11 bp, Homo sapiens
tttgcatcatc

SEQ ID NO: 180 - DNA, 11 bp, Homo sapiens
ttgacctgatt

SEQ ID NO: 181 - DNA, 11 bp, Homo sapiens
ttgacttgatt

SEQ ID NO: 182 - DNA, 11 bp, Homo sapiens
tattgtagatc

SEQ ID NO: 183 - DNA, 11 bp, Homo sapiens
tattgcagatc

SEQ ID NO: 184 - DNA, 11 bp, Homo sapiens
ccttttccctt

SEQ ID NO: 185 - DNA, 11 bp, Homo sapiens
cctttcccctt

SEQ ID NO: 186 - DNA, 11 bp, Homo sapiens
cttatgcagat

SEQ ID NO: 187 - DNA, 11 bp, Homo sapiens
cttatacagat

SEQ ID NO: 188 - DNA, 11 bp, Homo sapiens
aatacggtcat

SEQ ID NO: 189 - DNA, 11 bp, Homo sapiens
aatacagtcat

SEQ ID NO: 190 - DNA, 11 bp, Homo sapiens
ggaggtatgaa

SEQ ID NO: 191 - DNA, 11 bp, Homo sapiens
ggaggcatgaa

SEQ ID NO: 192 - DNA, 11 bp, Homo sapiens
agtccgtttcc

SEQ ID NO: 193 - DNA, 11 bp, Homo sapiens
agtccatttcc

SEQ ID NO: 194 - DNA, 11 bp, Homo sapiens
tctgccaaaga

SEQ ID NO: 195 - DNA, 11 bp, Homo sapiens
tctgcgaaaga

SEQ ID NO: 196 - DNA, 11 bp, Homo sapiens
acccattgttc

SEQ ID NO: 197 - DNA, 11 bp, Homo sapiens
acccactgttc

SEQ ID NO: 198 - DNA, 11 bp, Homo sapiens
gctactggctg

SEQ ID NO: 199 - DNA, 11 bp, Homo sapiens
gctaccggctg

SEQ ID NO: 200 - DNA, 11 bp, Homo sapiens
gaaatcacccg

SEQ ID NO: 201 - DNA, 11 bp, Homo sapiens
gaaattacccg

SEQ ID NO: 202 - DNA, 33 bp, Homo sapiens
gctctactgtcatttctaaccataatctcttta

SEQ ID NO: 203 - DNA, 27 bp, Homo sapiens
gcttcatatgatgaagggtaatgtggt

SEQ ID NO: 204 - DNA, 18 bp, Homo sapiens
tgtctttcagtatctctt

SEQ ID NO: 205 - DNA, 19 bp, Homo sapiens
tgtctttcaatatctcttc

SEQ ID NO: 206 - DNA, 444 bp, Homo sapiens
catttagtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcaatatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaa

SEQ ID NO: 207 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaacagaaggggtctgtctggctgggcttgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaatatgaactcaaaggaggtaagcaaaggggtgtgtgcgattctttgctactggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 208 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcttgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattcctcatagaatatgaactcaaaggaggtaagcaaaggggtgtgtgcgattctttgctactggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 209 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcttgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaacatgaactcaaaggaggtaagcaaaggggtgtgtgcgattctttgctactggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 210 - DNA, 837 bp, Homo sapiens
caaaaattagctaggtgtggtggtatgcacctataatctcagctacccaggaggctgaggcaggagaatcacttgaacctggaggcagaggttgcagtgagccgagacgcaccattacactccagcctgggtgacagagtgagattccatctcaaaaaaaaaaaaaaaaaattatgcctttttgaagcacatacattttataacatacaactgaatcccttattatattattagttttgatttaatgttttcaaaccatctcccctgatatttctgggagatgggaaacatgttttcttacacctcttgcattccattctcaactcccaactgtcttactgcaatgaacacttaataagaaacagtcaattggtcaattgattgggcaacaggctaaacacactcattccttgtctgttcccacttctttctttactttcccttcctgagtaacgtatcctaaagtcattaggtgggtggcagccagatggtggccacacattaaggtagaaaagagagtgtcatgatggttccaagtcagagacctagtagggtgaggatcaagtaggtgttcacgtggagaaacagcccggcctgtgtgtgggagtccaagcaagcagagaaaatgtcgacacagaggggtggcctgaaaaagcagccagagcctaaacagggcatggagaacatatttagggcatgaggtgaggagggcatccatgagtgggaagggatgggtgaggtttcactacataaaggggattgatgaaataagtaaataaagtatactggaagccaggtgtgtcacttttgcagaaaagagtcatggattcagaaagg

SEQ ID NO: 211 - DNA, 837 bp, Homo sapiens
caaaaattagctaggtgtggtggtatgcacctataatctcagctacccaggaggctgaggcaggagaatcacttgaacctggaggcagaggttgcagtgagccgagacgcaccattacactccagcctgggtgacagagtgagattccatctcaaaaaaaaaaaaaaaaaattatgcctttttgaagcacatacattttataacatacaactgaatcccttattatattattagttttgatttaatgttttcaaaccatctcccctgatatttctgggagatgggaaacatgttttcttacacctcttgcattccattctcaactcccaactgtcttactgcaatgaacacttaataagaaacagtcaattggtcaattgattgggcaacaggctaaacacactcattccttgtctgttcccacttctttctttactttcccttcctgagtaacttatcctaaagtcattaggtgggtggcagccagatggtggccacacattaaggtagaaaagagagtgtcatgatggttccaagtcagagacctagtagggtgaggatcaagtaggtgttcacatggagaaacagcccggcctgtgtgtgggagtccaagcaagcagagaaaatgtcgacacagaggggtggcctgaaaaagcagccagagcctaaacagggcatggagaacatatttagggcatgaggtgaggagggcatccatgagtgggaagggatgggtgaggtttcactacataaaggggattgatgaaataagtaaataaagtatactggaagccaggtgtgtcacttttgcagaaaagagtcatggattcagaaagg

SEQ ID NO: 212 - DNA, 602 bp, Homo sapiens
catagacaagggtgagtccttcagtacttagagaaaattcaagagtgactttaaattccccacttcaaatatattctctgttttcttgtctttcccttaagacatctctgaatagcttccttcaactgccagtgaaagatagcaggcctgatttcattggacacaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaata

SEQ ID NO: 213 - DNA, 602 bp, Homo sapiens
catagacaagggtgagtccttcagtacttagagaaaattcaagagtgactttaaattccccacttcaaatatattctctgttttcttgtctttcccttaagacatctctgaatagcttccttcaactgccagtgaaagatagcaggcctgatttcattggacgcaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggaaaattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaata

SEQ ID NO: 214 - DNA, 603 bp, Homo sapiens
agcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctgcacatttaactacaggtactgatctgttttgtgcttagatgttccccatcactgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaactt

SEQ ID NO: 215 - protein, 60 aa, Homo sapiens
SLAEDEEWKRIRSLLSPTFTSGKLKEMFPITAQYGDVLVRNLRREAEKGKPVTLKDIFGA

SEQ ID NO: 216 - DNA, 603 bp, Homo sapiens
agcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcacactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaactt

SEQ ID NO: 217 - DNA, 778 bp, Homo sapiens
cagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaaaaagttcctaaaatttggtttcttagatccattatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcatt

SEQ ID NO: 218 - DNA, 778 bp, Homo sapiens
cagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccgttatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcatt

SEQ ID NO: 219 - DNA, 778 bp, Homo sapiens
cagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccattatttctctcaataagtatgtgggctattatttctttctctctttttaaaaataactgctttcttgacatataattcacatatcgtataattcatccacttaaaaggtacaatttcattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcatt

SEQ ID NO: 220 - DNA, 778 bp, Homo sapiens
cagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccattatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcagtctagagccctcacacactttagctgtcaacaccccaccacaaaccccactgccctaagcatccaataatcaactttctgcctctatagatttgcctattctggacacttcatagaaataatatcatt

SEQ ID NO: 221 - DNA, 670 bp, Homo sapiens
agaaggtgccattgatctcactgctgtagtggtgtttcctatgtatagacctgcccttgctcagtcgccggcctgaaagaagggcaaacatgataaaaggaatgggttccagttgagaatcatgatgttcttattcttattactggtagagaaaattataattgctccaggtaaagtttgcattttcaatgatttccttttgttcgttttgtttttcccacagtactctttccattccttaccccagtttttgaagcattaaatgtctctctgtttccaaaagataccataaattttttaagtaaatctgtaaacagaatgaagaaaagtcgcctcaacgacaaacaaaaggtaaaatctgatggtggttaaatgacgatgtttaggttttgataaatttagattttatacacatgatagagcatgtatctgtatttttaaaaataaagacagagaacttatgtttagaacaagagaagccatttggtagaaataaagaaggagattggggaaggagatgagaatgagtcagagagatagcatttaaaacttgaaatcaggcacaacaattagtatgtcatgatataaacagtattgagataaaattttaccacttctcttccctttaataaattgtcaaaggataaagtttcctgtttgaaaatatatt

SEQ ID NO: 222 - DNA, 670 bp, Homo sapiens
agaaggtgccattgatctcactgctgtagtggtgtttcctatgtatagacctgcccttgctcagtcgccggcctgaaagaagggcaaacatgataaaaggaatgggttccagttgagaatcatgatgttcttattcttattactggtagagaaaattataattgctccaggtaaagtttgcattttcaatgatttccttttgtttgttttgtttttcccacagtactctttccattccttaccccagtttttgaagcattaaatgtctctctgtttccaaaagataccataaattttttaagtaaatctgtaaacagaatgaagaaaagtcgcctcaacgacaaacaaaaggtaaaatctgatggtggttaaatgacgatgtttaggttttgataaatttagattttatacacatgatagagcatgtatctgtatttttaaaaataaagacagagaacttatgtttagaacaagagaagccatttggtagaaatacagaaggagattggggaaggagatgagaatgagtcagagagatagcatttaaaacttgaaatcaggcacaacaattagtatgtcatgatataaacagtattgagataaaattttaccacttctcttccctttaataaattgtcaaaggataaagtttcctgtttgaaaatatatt

SEQ ID NO: 223 - DNA, 826 bp, Homo sapiens
agataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcatcatcctggccctgtataagatacatatcagaatgaaaaccactcccagtgtgactttgaattgcttttccattttttcttcttgggattagagagcttcacttagatttcatctaagctgtgatgttgtacgttgacctgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagc

SEQ ID NO: 224 - DNA, 826 bp, Homo sapiens
agataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcgtcatcctggccctgtataagatacatatcagaatgaaaaccactcccagtgtgactttgaattgcttttccattttttcttcttgggattagagagcttcacttagatttcatctaagctgtgatgttgtacgttgacttgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagc

SEQ ID NO: 225 - DNA, 616 bp, Homo sapiens
caggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgcagatccccttgaaattagacacgcaaggacttcttcaaccagaaaaacccattgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaatgtactgcatgagtagttggtgattttgtacattcattgagctctcccagagtctgtgtagagtgttgtgcattatgtagtataaaggaggtgaccaggtaagtgacagataggtagactcagcttctctgcttctcataggactacctctacccacctctagttagcatta

SEQ ID NO: 226 - DNA, 624 bp, Homo sapiens
gattaagcttttcatgattcctcatagaacatgaactcaaaagaggtcagcaaaggggtgtgtgcgattctttgctattggctgcagctatagccctgcctccttctccagcacataaatctttcagcagcttggctgaagactgctgtgcagggcagggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggcccctttccccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcaga

SEQ ID NO: 227 - DNA, 624 bp, Homo sapiens
gattaagcttttcatgattcctcatagaacatgaactcaaaagaggtcagcaaaggggtgtgtgcgattctttgctattggctgcagctatagccctgcctccttctccagcacataaatctttcagcagcttggctgaagactgctgtgcagggcagggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatacagatctatgaatgaataaataagcatttctcccatccaccttctaattttggtgactaggagggtttagggacagcatttggtagtgggaatgatttgattagcttagatctgacgaagactaatcaatgaaaacatggcagcggcaga

SEQ ID NO: 228 - DNA, 626 bp, Homo sapiens
gaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacagtcattgctgtctccaaccttcaccagcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctg

SEQ ID NO: 229 - protein, 60 aa, Homo sapiens
SVFTNRRSLGPVGFMKSAISLAEDEEWKRIQSLLSPTFTSGKLKEMFPIIAQYGDVLVRN

SEQ ID NO: 230 - DNA, 626 bp, Homo sapiens
gaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacggtcattgctgtctccaaccttcaccagcggaaaactcaaggaggcatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctg

SEQ ID NO: 231 - DNA, 626 bp, Homo sapiens
gaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacggtcattgctgtctccaaccttcaccagcggaaaactcaaggaggtatgaaaataagatgagtcttaattagaaatgtaaagaatgaatctggggacaggtagaaagtaagatcacagtccatttccaaggggtagtccactgagttcgagcttcctaaaaatggtcttttatctttatgtacagaaaagacatcacaaaattcattacaaaatgtcacttactgctccatgctggagaaagccatatccttctgggacttgagtctg

SEQ ID NO: 232 - DNA, 623 bp, Homo sapiens
attggacatgatagctagatttgtttcaggaaaacatcctgctttccaaggatttagatgaatgtttttgttcactggtgactcaggtaacacgtcttcaagaagccatagggaggttgagggagggaagtcaagaagggaggttgaggactgcacttttgatttacttctgacttcacgagtcactttctgcgaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaagtattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacata

SEQ ID NO: 233 - DNA, 616 bp, Homo sapiens
caggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgtagatccccttgaaattagacacgcaaggacttcttcaaccagaaaaacccactgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaatgtactgcatgagtagttggtgattttgtacattcattgagctctcccagagtctgtgtagagtgttgtgcattatgtagtataaaggaggtgaccaggtaagtgacagataggtagactcagcttctctgcttctcataggactacctctacccacctctagttagcatta

SEQ ID NO: 234 - protein, 59 aa, Homo sapiens
MRFALMNMKLALIRVLQNFSFKPCKETQIPLKLDTQGLLQPEKPTVLKVDSRDGTLSGE

SEQ ID NO: 235 - DNA, 830 bp, Homo sapiens
tggtcacccaccatgtgtacagtaccctgctagggtccagggtcatgaaagtaaataataccagactgtgcccttgaggaactcacctctgctaagggaaacaggcacagaaacccacaagggtggtagagaggaaataggacaataggactgtgtgagggggataggaggcacccagaggaggaaatggttacatctgtgtgaggaggttggtaaggaaagactttaatagaaggggtctgtctggctgggcttgcaaggatgtgtaggagtcatctagggggcacaagtacactccaggcagagggaattgcatgggtaaagatctgcagttgtggcttgtggggatggatttcaagtattctggaatgaagacagccatggaaacaagggcaggtgagaggatatttaagaggcttcatgccaatggctccacttcagtttctgataagaactcaggttccgtggactccctgataaaactgattaagttgtttatgattccccatagaatatgaactcaaaggaggtaagcaaaggggtgtgtgcgattctttgctaccggctgcagctgcagccccacctccttctccagcacataaacatttcagcagcttgacctaagactgctgtgcagggcagggatgctccaggcagacagcccagcaaacaacagcacacagctgaaagtaagactcagaggagacagttgaagaaggcaagtggcgatggacctcatcccaaatttggcggtggaaacctggcttctcctggctgtcagcctggtgctcctctatctgtcagtaactgtccagattcctctcctctgt

SEQ ID NO: 236 - DNA, 775 bp, Homo sapiens
ggagtgacctgattttccaggtgctgtctgtcacccctttctttgactaggaaagggaactccttgaccccttgcgcttctcaagtgaggcaatgcctcgccctgcttcggcttgtgcacagcacgctgcacccactgtcctgcacccactgtctggcactccctagtgagatgaacccggtacctcagatgaaaatgcagaaattacccgtcttctgtgtcactcacactgggagctgtagaccggagctgttcctattcggccatcttggctccaccccccgagttttggcttttaattgaaagtgtattgatgtgggaaggagataatgccatgcatttatgagcacatattagagggtctgagacaatgcatgtgataaaaggtcacctaaggaagaaaaaaagaacaagggaagacactggaaagaacgtgatgctgggagtccctgggccaccaaagtctggagaaaagtggtaaccacaaggctcccagcctagtttcactgaagacctcgacactaggtgacttatgggatccttggtaggacaagccttgaaaagttttacaatagcaaatgtggacgttgtcagaaccaaatgatgtcacgtgtgtatttgtgtgtgtgtgtcagtgtgtgtgtttaaaaatcatgacaaataaagcaggctgtgaagaggggattcccatgctcgtgtgcctgataacacaactatcacaaacgctttgcgaaaccacaagtttgcacaaaggcaatcccaaccttacacaaaa

SEQ ID NO: 237 - DNA, 25 bp, Homo sapiens
ttgttgggaaatgttttgtcctatc

SEQ ID NO: 238 - DNA, 24 bp, Homo sapiens
acagggagttgaccttcatacgtt

SEQ ID NO: 239 - DNA, 33 bp, Homo sapiens
tcagggtctctggaaatttgacacagagtgcta